(19) United States
(12) Patent Application Publication
(10) Pub. No.: US 2024/0110251 A1
(43) Pub. Date: Apr. 4, 2024

(54) CULTURE-FREE BIPHASIC APPROACH FOR RAPID DETECTION OF PATHOGENS FROM WHOLE BLOOD

(71) Applicant: THE BOARD OF TRUSTEES OF THE UNIVERSITY OF ILLINOIS, Urbana, IL (US)

(72) Inventors: Rashid BASHIR, Champaign, IL (US); Anurup Ganguli, San Diego, CA (US); Jongwon Lim, Savoy, IL (US); Ariana Mostafa, San Diego, CA (US); Enrique Andres Valera Cano, Champaign, IL (US); Matthew Wester, Champaign, IL (US); Jacob Berger, San Diego, CA (US)

(21) Appl. No.: 18/372,576

(22) Filed: Sep. 25, 2023

Related U.S. Application Data
(60) Provisional application No. 63/410,534, filed on Sep. 27, 2022.
(60) Provisional application No. 63/525,600, filed on Jul. 7, 2023.

(51) Int. Cl. C12Q 1/689; C12N 15/10; C12Q 1/6895; C12Q 1/70; G01N 1/44

(52) U.S. Cl. CPC C12Q 1/689; C12N 15/1003; C12Q 1/6895; C12Q 1/70; G01N 1/44

(57) ABSTRACT

Described herein are systems and methods which utilize an array of wells to rapidly detect target analytes from whole blood samples, even at very low concentrations. The provided systems and methods dry the blood sample to ensure a fluidic network forms in one or more dry blood sample islands and liquid with reagents useful for detecting nucleic acid sequences from a target analyte, including by amplification reactions that are bi-phasic because of the interaction between the solid dried blood sample phase and the liquid phases, including liquid having reagents that are able to fluidically and diffusively exchange with the fluidic network within the dried blood sample phase.

### CROSS REFERENCE TO RELATED APPLICATIONS

This application claims benefit of priority to U.S. Provisional Pat. App. Nos. 63/410,534 filed Sep. 27, 2022, and 63/525,600 filed Jul. 7, 2023, each of which are incorporated by reference herein to the extent not inconsistent herewith.

### STATEMENT REGARDING FEDERALLY SPONSORED RESEARCH OR DEVELOPMENT

This invention was made, at least in part, with United States governmental support awarded by the National Institutes of Health award no. 1R21AI146865A. The United States Government has certain rights in this invention.

### BACKGROUND OF INVENTION

Detection of low concentration pathogens is important in many applications, including food samples, environmental samples and patient samples, including blood, saliva and urine, and remains a challenge for medical diagnostic professionals. Many diagnostic techniques common today are methods which have been utilized for decades with minimal improvements in efficacy and efficiency. Currently, many diagnostic tests rely on cell cultures, microscopy or immunoassays which are lacking in sensitivity, costly and time consuming. Additionally, many common testing techniques are directed to single pathogens or conditions, requiring multiple tests to make a diagnosis. These constraints often force pathologists to rely on experience and guesswork, sometimes resulting in misdiagnoses or the overreliance on antibiotics when they may not actually be necessary, which in turn contributes to drug resistance.

For example, diagnosis of sepsis, a bacterial infection of the blood resulting from dysregulated inflammatory response, remains a challenge for medical professionals to quickly diagnose. The most common test for sepsis is a blood culture followed by nucleic acid amplification, which requires days to a week for a bacteria to grow to detectable quantities and has a high false negative rate. The time required for testing is problematic, as within days of the onset of infection a patient may progress into advanced stages leading to organ failure, causing sepsis to have high morbidity and mortality rates. As a result, sepsis remains one of the highest cost patient diagnoses.

Even as technology surrounding nucleic acid amplification techniques have advanced, significant hurdles remain in rapid, low cost identification of pathogens in bodily fluids. While tests have been developed for mRNA biomarkers and pathogen nucleic acids, current RNA/DNA based techniques often require expensive equipment and are directed towards single pathogens, as multiplexing increases costs. The nucleic acid tests also often require costly and time consuming purification steps before the sample may be analyzed. For detection of low concentrations of pathogens, nucleic acid testing is often preceded by a bacterial culture, requiring days to a week of culture time, thereby significantly increasing runtime.

As can be seen from the foregoing, there remains a need in the art for sensitive, rapid diagnostic tests based on nucleic acid detection, including by amplification, that are sufficiently sensitive to detect pathogens in very low concentrations within fluid samples within hours or less. Further, multiplexed testing techniques, which can accurately detect a panel of potential pathogens directly from the sample, are desirable.

### SUMMARY OF THE INVENTION

Described herein are systems and methods which may be configured in a microarray configuration to facilitate testing for pathogens directly from fluid, including liquefied solid samples, body fluids, food samples, environmental samples, with nucleic acid detection, including amplification techniques, to accurately and rapidly detect target analytes, such as pathogens or cell-indicating markers, in fluid samples, even in very low concentrations. Particularly preferred samples are whole blood samples from an individual, such as a human, where desirably identification of presence or absence of one or more target analytes is desired. The provided systems and methods dry the fluid sample, including a solid or semi-solid sample that has been liquefied, to deposit the total volume or a fraction of the volume in a dried sample island. The process of pixilation provides a platform where any number of wells contain any number of dried sample islands. By a specially configured bi-phasic reaction, a liquid with reagents specific for detection of a target analyte is applied to the dried sample island(s) nucleic acid detection is performed, such as by amplification, on each dried sample island to detect the presence of even a single individual target analyte and provide a quantitative analysis of the amount of target analyte within the sample. The systems and methods are robust, and ensure that reagents required for detection, such as amplification or crispr enzyme, reach each individual dried sample island (e.g., each well), and that reliable detection occurs, even approaching a single target analyte in a well. The technique is compatible with various bodily fluids, including blood, urine, saliva, sputum, etc. Similarly, the technique is compatible with solid and/or semi-solid samples that have been liquefied, including tissue, stool, food, environmental, and the like.

The described systems and methods may also be applied to water, liquid food, and solid food samples that is liquefied to a slurry-like paste or liquid. Testing of food samples to detect pathogens directly without nucleic acid amplification provides numerous benefits and has been seen as a holy grail in the food testing industry.

The systems and methods provided herein facilitate rapid and efficient detection of multiple target analytes. For example, a plurality of nucleic acid sequences or pathogen biomarkers may be capable of being detected for a run-time that is less than or equal to 45 minutes, greatly reducing the time required for an accurate diagnosis and allowing for treatment to occur much more quickly when compared to conventional testing techniques. Further, the provided techniques are multiplexible and facilitate detection of multiple pathogens with a single assay, reducing costs and leading to more accurate diagnoses.

The systems and methods are particularly suited for blood-based diagnostics because large blood volumes can be rapidly dried resulting in inactivation of the inhibitory components in blood. Further thermal treatments then generate a physical micro and nanoscale fluidic network inside the dried whole blood matrix to allow access to target nucleic acid from liquid supernatant overlaying the dried blood sample. The amplification enzymes and primers from the liquid supernatant initiate the reaction within the dried blood matrix through these networks, thereby avoiding any need for conventional nucleic acid purification. High heme background from the blood sample is confined to the solid phase, while amplicons are enriched, including in the clear liquid supernatant overlaying the solid phase, giving fluorescence change comparable to purified DNA reactions.

In this manner, without any significant or substantial sample processing steps, rapid and efficient diagnostics of whole blood is available with a robust limit of detection (LOD). The diagnostics may relate to a pathogen or a disease state. Pixilation of whole blood samples into smaller separated islands provides a convenient array platform where multiple blood samples may be independently tested and evaluated on one support substrate. In combination with optional electric-field lysing, the platforms provided herein are amenable to rapid, reliable and cost-effective diagnostics of whole blood.

Provided herein are methods of detecting one or more target analytes in a blood sample. The method may comprise the steps of: applying a blood sample to a substrate; thermally treating the blood sample to dry the blood sample to generate a dried blood sample island formed and having a fluidic network inside the dried blood sample island; applying a liquid having a reagent suspended in a liquid buffer to the dried blood sample island, wherein the reagent is used for nucleic acid detection, wherein the liquid transits the fluidic network to access a target nucleic acid if present in the dried blood sample island; diffusing a nucleic acid from the dried blood sample island to a supernatant liquid having the applied reagent. The supernatant liquid may correspond to at least a portion of the applied liquid having the reagent suspended in the liquid buffer. A target nucleic acid, if present in a dried blood sample island, is detected from a bi-phasic reaction, which occurs in both the dried blood sample island and in the supernatant liquid having the reagent suspended in the liquid buffer. In this manner, the combination of the dried and liquid phases, with the dried phase having a fluidic network to facilitate movement, such as diffusion, of components between the two phases together provide the ability to reliably detect the one or more target analytes in the blood sample.

The method may further include pixelating and/or electric field lysis aspects. For example, to facilitate the ability to test multiple sub-samples from a larger liquid sample, the larger sample may be “pixelated” into smaller sample sizes, including in an array configuration that may further facilitate high-throughput screening and multiplexing for detection of a plurality of unique target agents. In the array configuration, there is an ordered spacing and location of dried blood sample islands, such as in an array of microwells, with each well able to contain a liquid volume, including, for example, liquid volumes between 1 μL to 1 mL, including between about 50 μL and 250 μL, or about 100 μL.

The method may further comprise applying an electric field to the dried sample island to electrically lyse a biological material and release nucleic acid from the biological material. Examples of “biological material” include, but are not limited to, a pathogen selected from the group consisting of: bacteria, virus, fungus, and a cell having a marker for a disease condition, such as a cancer cell or any disease condition that can be identified by a unique nucleic acid sequence.

The method is compatible with a range of detection schemes. For example, the detection may be by optical detection, including a fluorescence detection of a probe or marker specific to the amplicon that reflects presence of a target analyte. The detection may be by electrical detection, wherein the amplicon that reflects presence of a target analyte has a unique electrical parameter, including, for example, FET detection using electrodes.

The methods provided herein are particularly useful for a blood sample, because the method facilitates confinement of heme background interference to the dried blood sample island.

The reagent in the applied liquid may comprise amplification enzymes and primers selected to amplify a target nucleic acid sequence of the target analyte. Preferably, the amplified nucleic acid sequence is specific for a target analyte so that positive detection of the resultant amplicon provides unique identification of one target analyte in the blood sample.

In an aspect, the target nucleic acid is amplified by the reagent into amplicons in the dried blood sample island, wherein the amplicons diffuse from the dried blood sample island to the supernatant liquid that covers the dried blood sample island. In this manner, the amplicons are detected by a change in fluorescence of the supernatant.

The method is compatible with a range of amplifications techniques, including, but not limited to, an isothermal reaction (including a loop-mediated isothermal amplification reaction or a recombinase polymerase amplification (RPA)); or a CRISPR-based amplification.

The target analyte may correspond to one or more pathogens.

The method may be characterized as having a limit of detection (LOD) of the target analyte in whole blood as small as 1 cfu per blood sample starting volume. The blood sample starting volume may be described in relative terms as a “large” blood sample, such as a blood sample starting volume of up to 10 mL, or between 1 mL and 10 mL. Again, for larger blood sample volumes, the pixilation into small sub-volumes of blood sample, may be helpful to identify low concentrations of target analyte (such as less than about 10 cfu/mL).

The methods provided herein are advantageous because there is minimal to no processing of the sample, unlike conventional methods that often require a cell culture, including to grow pathogen to a level that is reliably detectable. Accordingly, the methods provided herein may be characterized by a total test time to detection, such as wherein the target analyte is detected in a total test time that is less than 2.5 hours.

The blood sample may be unprocessed whole blood. In other words, a whole blood sample may be obtained, such as by a blood draw, and applied directly to the substrate, without any intervening processing steps. Of course, the method is compatible with some intervening steps inherent in conventional blood collection. For example, the blood may be drawn in the presence of an anti-coagulant to avoid undue blood clotting where the blood may not reliably be applied to cover at least a portion of a substrate to facilitate, for example, pixilation. The blood draw may utilize a blood collection tube having one or more additives (e.g., EDTA, sodium citrate, heparin) designed to stabilize and preserve the blood sample for subsequent analysis. The collection tube may be a vacutainer tube. The blood sample may then be collected from the collection tube and applied to the substrate. The “unprocessed” blood sample is not intended to necessarily exclude the steps associated with whole blood collection, but instead to exclude those steps that are time consuming or process intensive, such as culturing, centrifuging, and the like.

The method may further comprise the step of lysing the applied blood sample by applying an electric field to the blood sample island(s). In this manner, pathogens may be reliably lysed, including bacterial cells.

As discussed, the blood sample may be pixelated into an array of blood sample islands, and the electric field is applied to the array of blood sample islands. In this manner, multiple independent sample islands can be independently tested, or repeat tested, thereby improving accuracy and reliability.

The method may further comprise the steps of: applying a red blood cell (RBC) lysis buffer to lyse at least a portion of a population of RBCs in an unprocessed whole blood sample; and mechanically and/or electrically lysing a pathogen in the whole blood sample, including a pathogen that is a bacteria. In this manner, there may be at least two unique lysing protocols, a chemical lysing step for the RBCs (e.g., concentrated ammonium-chloride based buffer that minimally impacts nucleated cells) and an electrical and/or mechanical system to electrically and/or mechanically lyse other cells in whole blood, including a pathogen of interest, including bacterial cells.

The thermally treating step is of importance to ensure the dried blood sample island has a suitable internal network of passages to facilitate exchange of components between the solid phase (e.g. blood) and the liquid phase (e.g., liquid with reagent or the “liquid supernatant”). The thermally treating step may comprise elevating a temperature to between 65° C. and 97° C. for an elevation time of between 1 minute and 20 minutes to generate a porosity in the resultant dried blood samples for access by the reagents and diffusion of an amplified target nucleic acid from an interior of the dried blood sample to the liquid supernatant positioned outside the dried blood sample for ease of detection. The temperature and elevation time may be selected to achieve a porosity in the dried blood sample between 30% and 85%, including between 50% and 65%, of sufficient size for diffusion to occur of the relevant components, including the resultant amplicons, from the interior of the dried blood sample island, to the surrounding liquid supernatant.

The electric field may be applied after the liquid is introduced to the dried blood sample to lyse at least a portion of the dried blood sample island. In his manner, the liquid, formed from an ionic buffer, which has diffused or flowed into the dried blood sample island's network, helps ensure the electric field is sufficiently strong in the dried blood sample islands to lyse pathogens. Optionally, a chemical lysing agent is applied to the dried blood sample after the step of applying the electric field. The two different lysing platforms helps ensure adequate lysing of the components of whole blood.

The step of applying the electric field may comprise energizing an electrode pair. To facilitate appropriate electric field intensity to the blood sample, a first electrode may be positioned underneath a substrate that supports the dried blood sample; and a second electrode is positioned above or in the supernatant liquid.

As desired, any of the electrodes may have an increased surface area, thereby further increasing electric field intensity. One example of an increased surface area, is by having a roughened surface, wherein the roughened surface effectively increases the surface area of the electrode without increasing the overall base footprint of the electrode defined by the bulk perimeter of the electrode.

The fluidic network in the dried blood sample island can be filled with the applied liquid and reagent(s) to provide one or more localized regions having a local electric field intensity to increase a lysing efficiency as compared to fluid regions without the dried blood sample. In other words, for applied liquid and reagent not on or surrounding a dried blood sample island, the local electric field may be less as there is no need to lyse a biological material in such regions.

The methods provided herein are particularly useful for sepsis-related applications. Accordingly, the detecting the target analyte step may be used to select an antibiotic treatment therapy in an individual requiring treatment of a blood stream infection. For example, the method may further comprise the step of identifying for the detected one or more target analytes a bacteria that is an antibiotic resistant bacteria and/or an antibiotic-susceptible bacteria. In this manner, the method may further comprise the step of providing to a patient in need of treatment a specific type of antibiotic, including a mixture of antibiotics, tailored to the identified bacteria. The detecting the one or more analytes may be within 3 hours of the individual initially presenting with a blood infection symptom, thereby providing actionable information for a patient at risk of developing sepsis before the patient develops serious symptoms. Such a rapid time for detection is provided by the configuration of instant methods and devices having the dried blood sample islands with microfluidic bi-phasic reactions that can reliably detect a plurality of unique target analytes at low target analyte concentrations and with minimal to no blood sample processing. The actionable information may be selection of one or more antibiotics tailored to the detected target analyte(s) and corresponding type of bacteria.

Also provided herein are methods of determining presence or absence of a pathogen in an individual that may have a blood stream infection. The method may comprise the steps of: obtaining a whole blood sample from the individual; thermally treating the whole blood sample to generate a dried blood sample island having a fluidic network inside the dried blood sample; lysing RBCs in the whole blood sample and mechanically or electrically lysing a pathogen in the whole blood sample to generate a lysed blood sample; introducing a liquid buffer having amplification reagents to the dried blood sample island, wherein the amplification reagents diffuse through the fluidic network to contact an interior portion of the dried blood sample; amplifying a target nucleic acid, if present, to generate amplicons in the interior portion of the dried blood sample; diffusing the amplicons from the interior portion of the dried blood sample to a liquid supernatant that surrounds the dried blood sample; and optically detecting amplicons in the liquid supernatant, wherein a positive optical detection corresponds to presence of a pathogen in the whole blood sample and a negative optical detection corresponds to absence of the pathogen in the whole blood sample. In this manner, the presence or absence of the pathogen in the blood stream of the individual is determined. The pathogen may correspond to an antibiotic-resistant or an antibiotic-susceptible bacteria. In this manner, a tailored antibiotic treatment is possible without having to undergo a time-consuming bacterial culture.

The methods provided herein are compatible with any of a range of pathogens, depending on the application of interest. Examples include a pathogen corresponding to a bacteria, a fungus, and/or a virus. As described, the method may be a multiplexible method so that multiple pathogens may be examined in one array. For example, each target pathogen may have a unique optical output, such as fluorescence output. Alternatively, different reagents may be applied to different wells.

The methods are compatible with large blood volumes by pixelating the lysed blood sample into a plurality of blood sample islands, wherein the plurality of blood sample islands are arranged in an array configuration. This provides a convenient platform for high-throughput screening and simultaneous diagnostics of a plurality of target analytes.

The lysing step may comprise applying an electric field after the liquid buffer is introduced to the dried blood sample to lyse at least a portion of the dried blood sample island and at least a portion of any pathogens therein. In this manner, the fluidic network inside the dried blood sample island is filled with liquid buffer, and an appropriate electric field intensity is positioned in the dried blood sample island. The lysing step may further comprise the step of chemically lysing at least a portion of the whole blood sample and/or the dried blood sample.

Also provided are methods of detecting one or more target analytes from a liquid sample, the method comprising the steps of: applying a liquid sample to a substrate, wherein the substrate has an array of wells; drying the liquid sample to generate a plurality of dried sample islands, with each sample island confined to a unique well; applying a reagent to each well, wherein the reagent is used for nucleic acid detection; eluting a nucleic acid from the dried sample islands with a liquid phase having the applied reagent; detecting a target nucleic acid if present in a dried sample island by a bi-phasic reaction, wherein the detecting is by electrical or optical detection; thereby detecting the one or more target analytes from the liquid biological sample.

“Eluting” is used broadly to refer to the ability of a material (e.g., nucleic acid) in a solid sample to contact liquid (e.g., reagent in the liquid). In the context of a dried sample, eluted nucleic acid refers to lysis of cells to expose nucleic acid to the liquid, including by thermal, chemicals or enzymes, or any technique that can reliably release nucleic acid from the sample. The eluted nucleic acid may continue to reside within the boundaries formed by the outer surface of the dried sample, such as be liquid that has penetrated the dried sample. The eluted nucleic acid may diffuse into the bulk liquid phase, referred herein as “supernatant” that surrounds the dried sample.

The reagent is used for the detecting step, and so may correspond to reagents useful for nucleic acid amplification or may be for a more direct form of detection that does not require amplification, such as by a crispr enzyme. The liquid sample may be dried multiple times, with reagents added between the repeated drying steps. The reagent may be added in the form of a reagent that is provided in a liquid, such as a reagent that is suspended in liquid and that is in intimate contact with the dried sample. The liquid sample may be a crude liquid sample, for example, a liquid sample that has not undergone any processing or purification. In any of the systems and methods described herein, one or more reagents may be introduced into the wells of the array before the liquid sample to be analyzed, after the liquid sample, or some reagents applied before and some after. In any of the described methods, each well of the array may be isolated from other wells, for example, by using one or more barrier layers, such as hydrophobic liquid layers, solid layer barriers and liquid-tight barriers between adjacent wells, thereby limiting or eliminating cross talk between each individual well. The reagents may be specially delivered to the wells, including by reagents released from the barrier layer under a force, such as a centrifugal force.

Any of the methods may further comprise forming microchannels and/or nanochannels in the dried sample islands to facilitate introduction of reagent-containing liquid into an interior portion of the dried sample islands. The eluted nucleic acid may remain in the microchannels and/or nanochannels, may diffuse from the microchannels and/or nanochannels to the liquid phase that is a supernatant to the dried sample islands, may be released directly from the solid to the liquid supernatant, or be a combination thereof. The nano and microchannels may form as part of the drying process, or may be actively induced such as by the use of degradable scaffolds over which the liquid sample is deposited, so that upon drying and degradation, a network of fluidic channels traverse the dried sample. Similarly, beads with reagents connected thereto may be used, so that reagent is introduced into the interior portion of the subsequently dried liquid sample.

Drying and/or eluting (e.g., thermal, chemical, enzymatic) of the sample produces a high surface area to volume ratio, including by producing channel-like structures with passages and/or openings of micro (e.g., less than 1000 μm dimension) and/or nano (e.g., less than 1 μm dimension) scale pores, such as diameters between 0.1 μm and 1000 μm, and any sub-ranges thereof. Such micro and nano vasculature and channels facilitates introduced reagent to access the pathogens and the DNA within the dried sample. Also, when wetted, the high surface area to volume ratio structures with micro and nano vasculature and channels allows for the DNA from the pathogens to escape the blood and access the reagents, such as the polymerase or crispr enzymes, in the supernatant (e.g., bi-phasic reaction). Either way, the combination of drying and introducing the reagents in the appropriate sequence allows for even a single DNA from the pathogen to be detected and/or amplified. Other detection modalities include by crisper enzymes, such as crispr-cpfl. The impurities and microscale debris/contaminants can be tethered as precipitate or solid phase as a result of drying (not mobile and cannot effectively mix with the reagents) and nanomolecules responsible for reactions (enzymes, dna, and the like) can freely roam around in the liquid phase owing to their small size and high diffusion coefficients in liquid. This allows reactions to proceed smoothly even in the presence of debris from unpurified samples.

In embodiments, the liquid sample is a biological sample. In an embodiment, for example, the liquid sample is minimally processed, including an unprocessed raw sample. Depending on the liquid sample of interest, some minimal processing may be desired. For example, a blood sample may be obtained with an anti-coagulant, to avoid unwanted coagulation. Viscous samples may be diluted with a lower viscosity solvent, thereby facilitating uniform liquid application over the array. For other samples having inherently desired liquid parameters, such as saliva, urine, etc., the obtained bulk sample may be applied directly to the wells. Preferably, the wells have sharp or sloped edges of varying angles so no liquid sample remains outside wells, thereby ensuring more robust capture of liquid containing a target analyte of interest. In an embodiment, the applying the reagent step is before or after the applying the liquid sample step. In an embodiment, the bi-phasic reaction comprises the dried sample island and the reagent suspended in a buffer fluid, wherein the buffer fluid is positioned over the dried sample island in the well.

The method may further comprise a step of forming high surface area structures in the plurality of dried sample islands and/or a reagent liquid positioned over the dried sample islands. The high surface area structures may be formed by mixing gas with the liquid biological sample before or during the drying step. The high surface area structures may comprise a reagent-connected bead suspended in the reagent liquid. The high surface area structures can be formed by degradable micro-nano substrates that will degrade post sample drying and create channels/cavities in the process. In this manner, micro and/or nano-structures may be placed onto the bottom of the well, liquid sample provided thereto that is dried. Upon degradation of the micro and/or nano-structures the remaining dried sample has passages therein.

The provided systems and methods utilize advanced techniques to ensure that enzymes or reagents are provided in each individual well, and thus nucleic acid amplification is performed on each of the dried sample islands. For example, molecules or enzymes may be covalently or non-covalently connected to the surfaces of beads and positioned in each well by various means including gravity or magnetism. This leads to the capability of detecting small concentrations, as small as individual target analytes or pathogens, by ensuring replication on small volume dried sample islands which represent a small fraction of the total volume of the liquid sample.

Any of the methods provided herein may further comprise a bead-based molecule delivery system, wherein the molecule(s) connected to the bead surface is used in the amplifying step. For example, the molecule may be an enzyme, including a polymerase. In an embodiment, the bead-based molecule delivery system comprises a liquid bi-layer application, wherein: a) a buffer mixture covers the dried sample, b) a carrier fluid having the bead-molecule suspended therein is disposed over the buffer mixture, and c) the method further comprising a step of forcing the bead-molecule into the buffer mixture, including settling by gravity or magnetic force for magnetic beads.

In an embodiment, for example, the bead-based molecule delivery system comprises a liquid tri-layer application, wherein: a) a buffer mixture covers the dried sample islands, b) an immiscible separating fluid that covers the buffer mixture, c) a carrier fluid having the bead-molecule suspended therein is disposed over the immiscible separating fluid, wherein the carrier fluid further comprises a surfactant that forms micelles around the bead-molecule to protect the molecule from denaturing, and d) the method further comprising a step of forcing the bead-molecule into the buffer mixture.

In an embodiment, the bead-based molecule delivery system comprises a liquid layer and frozen liquid layer application, wherein: a) a buffer mixture covers the dried sample islands, b) a carrier fluid having the bead-molecule suspended therein, wherein the carrier fluid has a freezing point less than a freezing point of the buffer mixture, and c) the method further comprising the steps of: i) freezing the buffer mixture by cooling the buffer mixture to a temperature that is below the buffer mixture freezing point and that is above the carrier fluid freezing point, ii) applying the carrier fluid on top of the frozen buffer mixture, iii) forcing the bead-molecule to the frozen buffer mixture-liquid carrier fluid interface, iv) thawing the frozen buffer mixture, and v) forcing the bead-molecule into the buffer mixture.

In embodiments, the high surface area structures comprise a microrelief structure having a molecule connected thereto, the method further comprising the steps of: a) aligning the microrelief structure with the array of wells; b) inserting the microrelief structure into a buffer mixture that occupies the wells and covers the dried fluid sample; and c) wherein the inserted microrelief structure introduces the molecule to the buffer mixture and prevents evaporation from the wells.

The systems and methods are capable of providing rapid and multiplexed testing to quickly identify or quantify multiple analytes in an unprocessed, or crude, sample. In some embodiments, for example, the testing may be completed in less than or equal to 45 minutes, or optionally, less than or equal to 30 minutes. In embodiments, the multiplexed methods and systems may test for more than one analyte, 10 or more analytes, or optionally, 100 or more analytes. The device may have a high number of wells, including between 1,000 to 10,000,000 wells or more that are homogeneously filled with a volume of between 1 μL and up to 10 μL to 100 μL, or between 1 μL and 1000 nL, over a rapid timeframe, including within 15 seconds or within about 60 seconds.

In an embodiment, the liquid sample undergoes minimal or no sample processing, for example, no purification or processing steps are performed on the liquid sample prior to drying, except application of a liquid to achieve desired volume and/or an anti-coagulant to avoid clots and minimize non-uniform dispersion in the wells. In embodiments, the method is rapid and highly-multiplexed. In embodiments, the method has a high sensitivity, with as low as 1 copy of a target nucleic acid per reaction well, in a total liquid sample volume of up to 10 mL. The liquid sample may undergo thermal lysing. The liquid sample may undergo chemical lysing, such as with an application of a chemical such as Triton×100 to selectively lyse red blood cells, thereby reducing overall cellular debris before drying.

The described systems and methods are versatile, and may be used for detection of nucleic acids in a variety of biological or non-biological liquid samples. The analyte of interest may be a pathogen (e.g. a bacterium, virus, fungus or mold), a cancerous or genetically altered host cell or other biological target containing nucleic acid. Further, various techniques may be used to analyze and/or quantify an amplified sample as described herein, including optical detection (e.g. imaging, fluorescent dyes), mechanical detection (e.g. resonant frequency, stress induced mechanical bending), and electrical detection, for example, using FETs.

In an embodiment, the provided method is used in an application selected from the group consisting of: a) cancer molecular screening; b) pathogen detection from body fluids, including for diagnosis of sepsis; c) treatment efficacy assessment; d) detection of pathogens from food and water samples; e) detection of rare cells in biological sample, including circulating tumor cells; and f) detection of cell-free DNA from a body fluid sample. The systems and methods described herein may detect pathogens from solid foods, liquid foods or both solid and liquid foods.

For example, the methods and systems provided herein are compatible with pathogen susceptibility testing to different drugs, including in a patient's blood. The approaches provided herein are configured so that after dispensing whole or diluted whole blood into the wells, a bacterial culture is performed within the wells for 1-2 doubling cycles to ensure that the wells with the single bacteria have at least 2-3 bacteria. The presence of the live versus dead bacteria can be detected by using optical/fluorescence based approaches. Then drugs are introduced on the array and the viability of the cells are again optically mapped and detected. Similarly in another approach, after a brief cell growth is performed, the sample can be split and one well array can be used for the drug susceptibility testing and the other for the pathogen detection testing.

The method and systems provided herein, wherein the liquid sample is selected from the group consisting of: whole blood; saliva; urine; sweat; throat swab; vaginal swab; sputum; drinkable fluid; edible food; and environmental samples. A sample more on the solid phase of the liquid-solid phase characterization, may be dissolved in a solution with a solvent, including water, for use in any of the methods or devices described herein. In embodiments, the nucleic acid amplification comprises PCR or isothermal processes, for example, LAMP or RT-LAMP. In embodiments, the one or more target analytes comprise a nucleic acid sequence indicative of a pathogen or a disease state.

In an embodiment, for example, the buffer fluid comprises: a) an amplification enzyme and primers for nucleic acid amplification of a target analyte; and b) the amplification occurs in both the solid phase dried sample and in the solution phase buffer fluid through simultaneous diffusion of enzymes and buffer components into the dried fluid sample and target nucleic acid into the buffer fluid.

In embodiments, each of the wells has a well volume selected from the range of 1 μL to 100 μL, 1 μL to 10 μL, 1 μL to 1000 nL, 1 μL to 1 nL, or optionally, 1 μL to 250 μL. In an embodiment, the method further comprises a step of thermally lysing biological cells contained in the liquid sample. In embodiments, the substrate is part of a microfluidic chip formed of silicon, glass, polymer, or plastic. In embodiments, the detecting step comprises optically detecting fluorescent output of each of the wells or electrically detecting an electrical parameter in each of the wells, for example, by measuring a change in pH in each well by ion sensitive field effect transistors (ISFET). In embodiments, the detecting step comprising mechanically detecting a parameter, such as resonant frequency or stress induced mechanical bending, for example, by using a quartz crystal microbalance or a MEMs cantilever.

In an embodiment, for example, the method further comprises the steps of: a) filling all wells with the fluid sample; and b) removing excess fluid sample that is not contained in the wells by introducing a gas over the wells at a pressure that is sufficient to remove excess fluid while fluid in the wells are maintained with the wells by capillary forces. In an embodiment, the gas is an inert gas, for example, inert with respect to the amplification reaction such as Nitrogen.

In an aspect, provided is a device for performing any of the methods described herein. The device may be functionally described as a ‘pixelated petri dish’—a large circular substrate with the wells, and the number of wells and size of wells selected to hold the entire fluid sample and be subdivided into the wells. For example, the device may further include an instrument reader configured to perform real time imaging of each pixel to examine output from the well, including fluorescent intensity change as an indication of amplification.

The sample may be from a fluid sample or may be from a solid sample, including food, environmental, animal, or the like, that is processed by the techniques herein by converting the solid into a liquid through various methods such as blending and/or homogenization.

Any of the methods may further comprise the step of thermally lysing the sample.

Any of the methods may further comprise the step of connecting a dried material to a hydrophobic substrate release surface; fluidically sealing the array of wells by providing the hydrophobic substrate onto a top portion of the array of wells, wherein the substrate release surface faces toward the array of wells; and releasing the dried material from the hydrophobic substrate to wells.

The dried material may comprise a lyophilized or dried biomolecule connected to a bead surface. The bead surface can provide a convenient handle, with the material covalently or non-covalently bonded to the bead surface in a well-controlled and well-defined manner. The bead may then connect the material to the hydrophobic substrate and be utilize to help facilitate release into the microwell. A magnetic bead may be magnetically released. A gravitational force may be used to release the bead and material to the microwell. The material may be chemically released, including by a release agent in the microwell supernatant phase that cleaves the bead from the hydrophobic substrate and/or the material from the bead surface.

The releasing step may comprise centrifuging the fluidically sealed array of wells and hydrophobic substrate to release the dried material from the hydrophobic substrate to the array of wells.

The dried materials may comprise Crispr-cpfl, Crisper-Cas, polymerases, ligases, or other enzymes to edit, add, delete, or modify nucleic acids, or any other materials targeted to the application of interest. See also, US Pat. Pub. Nos 2023/0042422 (“CRISPR Cascade Assay”) and 2023/0052518 (“Nuclease Cascade Assay”) for various detection assays, including CRISPR nuclease cascade and nuclease cascade assays, which are useful in the context of the instant invention for detection of nucleic acid sequences of interest, and which is specifically incorporated by reference herein.

At least 100 different target analytes are identifiable by sequential addition of Crispr-cpfl and corresponding sequential optical detection of a fluorescent signal generated by bound cpfl to a target analyte, including up to between 100 and 1000.

Also provided herein are systems for introducing a biomolecule to a plurality of wells in an array for target analyte detection, including for any of the methods provided herein. The system may comprise: a plate having a plurality of microwells, each microwell having: a well surface; a top portion that is physically accessible; and wherein adjacent microwells are separated by a separation surface; a hydrophobic substrate having a release surface; a biomolecule that is connected to the hydrophobic substrate release surface; wherein the hydrophobic substrate release surface with biomolecules connected thereto is configured to connect to the microwell separation surface and fluidically seal the microwells to prevent fluid transmission between different microwells filled with a fluid.

The biomolecule may be connected to the hydrophobic substrate release surface by a bead. The bead has a bead volume (V_{bead}) and the microwell has a microwell volume (V_{well}) and, optionally, V_{well}/V_{bead}>1000.

The system may have a plurality of hydrophobic substrates, wherein each hydrophobic substrate comprises a unique biomolecule that is different than any other hydrophobic substrate biomolecule, wherein the plurality of hydrophobic substrates provides multiplexed detection of target analytes.

Also provided is a system or device for practicing any of the methods described herein. Also provided are methods for practicing any of the systems or devices described herein.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIGS. 1A-1B. Process flow showing the principle for change in pH during LAMP reaction (FIG. 1A), and the associated measured signal from ISFET chip (red=positive, blue=negative) (FIG. 1B).

FIGS. 2A-2B. An ISFET chip with microwell array (FIG. 2A), and the USB version of a similar ISFET chip (FIG. 2B).

FIG. 3. Schematic showing the process flow of the USB-based molecular sensing technique.

FIGS. 4A-4B. Glass slide with dried blood (FIG. 4A), and glass slide interfacing the ISFET chip with patterned microwell array (FIG. 4B).

FIG. 5. Provides SEM images of dried blood samples.

FIG. 6. Provides SEM images of high surface areas structures generated by microbubbles formed by mixing gas with the liquid sample before or during the drying step.

FIGS. 7A-7B. Process flow of the dried liquid sample bi-phasic reaction. FIG. 7A. 1. Whole blood is dried at the bottom of a PCR tube. For non-thermophilic polymerase enzyme, 2a. nuclease-free water is added to the tube and the tube is heated at 95° C. to elute the bacterial DNA. 3a. reaction mix with the polymerase enzyme is added to the tube post-elution. For thermophilic enzyme, 2b. reaction mix with the polymerase is added directly to the dried blood. 3b. Thermal lysis of cells is carried out at 95° C. directly in the reaction mix. 4a. Lamp reaction is carried out at 65° C. in a commercial thermocycler for thermophilic polymerase, and at the appropriate temperatures when using thermophilic polymerase. FIG. 7B. LAMP amplification curve for the detection of E. coli O157:H7 strain using our dried blood biphasic reaction.

FIGS. 8A-8B. Comparison of the described dried blood reaction with other whole-blood based LAMP technique. FIG. 8A. Fluorescence intensity curves showing the amplification reaction of the three different reaction types. Blue=our dried blood bi-phasic approach. Red=single phase reaction with thermal lysis. Green=single phase reaction using a blood lysis buffer.¹² FIG. 8B. Threshold time bar graph of the corresponding splits in FIG. 8A. The dried blood-based biphasic reaction (blue) gave much shorter threshold times compared to other LAMP reactions using whole blood (red and green). The bi-phasic nature of the reaction reduces the effective concertation of contaminants in the reaction mix, thereby increasing the reaction efficiency.

FIGS. 9A-9B. Template accessibility test. FIG. 9A. 1. Whole blood is dried at the bottom of a 0.2 mL PCR tube. 2. Nuclease-free water is added to the tube and the tube is heated at 95° C. to lyse the cells and elute the bacterial DNA. 3a. In the first split, reaction mix is added on top of the water and LAMP reaction is carried out. 3b. In the second split, supernatant post-elution is taken out and mixed with the reaction mix. LAMP reaction was carried out at 65° C. FIG. 9B. Threshold time bar graph of the amplification reaction. Red=reaction with the supernatant only. Blue=Complete biphasic reaction (reaction with both the supernatant and the dried blood). The complete dried blood approach gave shorter threshold times indicating that not all template DNA is eluted in the nuclease-free water added.

FIGS. 10A-10B. Dried blood reaction compatibility testing. FIG. 10A. Real-time fluorescence curves of the LAMP amplification reaction using two strains of E. coli (gram-negative bacteria). FIG. 10B. Real-time fluorescence curves of the LAMP amplification reaction using two strains of Staphylococcus Aureus (gram-positive bacteria). The dried blood approach compatible with both gram-negative and gram-positive bacteria. The concentration for each pathogen was kept constant at 1.25e3CFU per reaction.

FIGS. 11A-11D. Primer cross-reactivity test. Real-time fluorescence curves of the LAMP amplification reactions used to validate the primer specificity. FIG. 11A. wzy primer set specific to the O157:H7 (non-drug resistant strain) of E. coli only. FIG. 11B. NDM-1 primer set specific to the drug-resistant strain of E. coli only. FIG. 11C. femA primer set specific to Staphylococcus Aureus regardless of the strain type FIG. 11D. mecA primers specific to the Methicillin Resistant S. aureus only.

FIG. 12. Stability of dried blood over time. Real-time fluorescence curves of the LAMP amplification reactions to test the dried blood stability. Blood dried up to 28 days were tested. The threshold times for the positive amplification at day 0 were found to be identical to the threshold times for positive amplification at day 28. No non-specific amplification was observed even with blood dried for 28 days.

FIGS. 13A-13C. On-chip blood loading. Microscopy images of dried blood in (FIG. 13A) 100 um, (FIG. 13B) 300 um, (FIG. 13C) 500 um wells. The well edges are clearly visible in all of the chip types indicating minimal cross-talk between adjacent wells. The filling was also observed to be uniform in the wells.

FIG. 14. Mechanism of polymerase lyophilization on beads. 1. Beads are added to a 1.5 mL centrifugation tube. 2. Polymerase enzyme solution is added to the tube with beads and mixed thoroughly by pipetting. 3. The mixture of beads and polymerase is lyophilized. The polymerase lyophilizes on the surface of the beads. 4. Prior to the amplification reaction, the appropriate carrier fluid is added each to lyophilized tube and mixed thoroughly before being pipetted into the reaction.

FIG. 15. On-chip process flow for a bi-layered fluid bead technique.

FIGS. 16A-16C. Tube-based bi-layer bead loading approach. FIG. 16A. Protocol of the bi-layered bead loading approach for the introduction of polymerase enzyme. 1-2. Blood is dried and eluted as per previous protocol. 3. Reaction mix without the polymerase enzyme is added on top of the eluted water. 4. Beads containing lyophilized polymerase enzyme, suspended in a mixture of a surfactant and mineral oil (or any other similar immiscible liquid such as hexane or toluene), is added on top of the reaction mix without the polymerase. 5a. The beads are pulled down from the mixture of Triton-X 100 and mineral oil via centrifugation. 5b. Magnetic beads are pulled down to the bottom using a strong magnet. FIG. 16B. 1.5 mL centrifuge tube containing beads mixed in mineral oil and Triton-X 100 added on top of water. FIG. 16C. The beads move down from the mixture of mineral oil and Triton-X 100 upon centrifugation (observed as brown bolus at the bottom of the tube).

FIGS. 17A-17B. Bi-layered approach enzyme delivery LAMP reaction. LAMP amplification curves using the bi-layer approach with (FIG. 17A) 40000 polystyrene beads per reaction, and (FIG. 17B) 4000 magnetic beads per reaction.

FIGS. 18A-18H. Microscopic validation of the bi-layered bead loading approach. FIGS. 18A-18B. Beads suspended in a mixture of mineral oil. Large clumps of beads are observed suggesting the tendency of beads to agglomerate in hydrophobic conditions. FIGS. 18C-18D. Beads in water after centrifugation. The beads are observed to be surrounded by a ring of mineral oil and are not available to interact with the template. LAMP reaction with this split yielded no amplification. FIGS. 18E-18F. Beads suspended in a mixture of mineral oil (or any other immiscible liquids such as hexane and toluene) and TritonX-100 (or any other surfactant). Clumps of beads, though smaller than in FIGS. 18A-18B, can be seen due to the non-homogenous nature of bead mixing in the mixture of mineral oil and Triton-X 100. The beads are stabilized by the presence of Triton-X 100 in the mineral oil. FIGS. 18G-18H. Beads in water after centrifugation. The beads appear in a single phase indicating that the lyophilized enzyme is free to interact with the template.

FIG. 19. On-chip process flow for the tri-layered bead loading technique.

FIGS. 20A-20C. Tube based tri-layer bead loading approach. FIG. 20A. Protocol of the tri-layered bead loading approach for the introduction of polymerase enzyme. 1-2. Blood is dried and eluted as per previous protocol. 3. Reaction mix without the polymerase enzyme is added on top of the eluted water. 4. Mineral oil is added on top to prevent the mixing of the reaction mix with the polar solvent (to be added in step 5). 5. Beads containing lyophilized polymerase enzyme, suspended in a solution of Triton-X 100 and ethanol (or any solvent that do not mix with the mineral oil, and is less dense than both water and the mineral oil), is added on top of the reaction mix without the polymerase. 6a. The beads are pulled down from the mixture of Triton-X 100 and ethanol solution via centrifugation. 6b. Magnetic beads are pulled down to the bottom using a strong magnet. FIG. 20B. Stacked layer of beads in ethanol and Triton-X 100 on top of mineral oil and the reaction mix with dried blood before centrifugation. FIG. 20C. Stacked layer of beads in ethanol and Triton-X 100 on top of mineral oil and the reaction mix after centrifugation. The brown color of the beads is no longer visible in the topmost layer after centrifugation.

FIGS. 21A-21B. Tri-layered approach enzyme delivery LAMP reaction. LAMP amplification curves of the tri-layer approach carried out using (FIG. 21A) 4000 polystyrene beads per reaction, and (FIG. 21B) 4,000,000 magnetic beads per reaction.

FIGS. 22A-22D. Microscopic validation of the tri-layered bead loading approach. FIGS. 22A-22B. Beads suspended in a mixture of TritonX-100 and ethanol. The beads are homogenously spaced with very few clumps. FIGS. 22C-22D. The beads appear homogenously mixed in a single phase indicating that the lyophilized enzyme is free to interact with the template.

FIG. 23. On-chip process flow for bead loading at sub-zero temperature.

FIG. 24. On-chip process flow for microrelief or micropillars-based amplification enzyme delivery.

FIG. 25 schematically illustrates the introduction of a lyophilized or a dried material (e.g., a biomolecule) on a substrate for homogeneous delivery to individual wells of a microwell array. The left panel illustrates wells of a microwell array filled with reagent. The middle panel illustrates a hydrophobic substrate with dried biomolecules connected thereto over the wells. The biomolecules are pulled down into the wells, such as by a gravitation force, centrifugal force, or magnetic force.

FIG. 26 is a schematic illustration of use of an enzyme, Crispr-cpfl, for multiplexed detection of nucleic acid targets with any of the digitized platforms provided herein. The left panel illustrates one unique target molecule per well, a configuration referred to herein as “sample partitioning”. Beads with Crisper-cpfl enzyme may be introduced, including by a process similar to that illustrated in FIG. 25, as shown in the middle panels. The introduction may be sequential, so that many different targets are analyzed by sequential analysis of a fluorescent signal, wherein a positive fluorescent signal from any well indicates the presence of the target corresponding to cpfl delivered (right panel).

FIG. 27. Biphasic reaction for pathogen identification. (Top panel) Blood culture-based PCR methods as current gold standard. (Top left) Diagnosis time is governed by blood culture time. (Top right panel) If the blood culture is positive, PCR is performed. (Panels c-h) Protocol workflow of our blood-processing module and following biphasic LAMP reaction for culture-free pathogen identification. (c) Red blood cell lysis using ACK lysis buffer. (d) Mechanical vortex for bacteria lysis and DNA extraction. (e) Direct drying of whole blood without purification to create dried blood matrix while inactivating the inhibitory elements in whole blood. Thermal lysis improves the porosity of micro- and nano-fluidic network within the dried blood matrix. (f-h) Biphasic LAMP reaction where (g) the solid phase of dried blood matrix acts as a substrate. The enzyme initiates LAMP amplification at a single temperature (65° C.). The amplicons diffuse out to (h) the liquid phase and bind to the fluorescent dye in clear supernatant, increasing the signal-to-noise ratio. Total turnaround time is 2.5 hours, including 1.5 hours of sample processing and 1 hour of LAMP reaction.

FIGS. 28A-28D. Biphasic reaction schematic and analysis of blood pre and post thermal lysis. FIG. 28A. Process flow schematic of biphasic reaction. Post drying, LAMP buffer reagents are added, and thermal lysis is conducted. Finally, primers and polymerase are added for the final reaction. Micro-nano fluidic channels are created during the thermal lysis heating step, so primers and polymerase may enter the blood matrix and find target DNA. FIG. 28B. SEM images of the blood cake before thermal lysis. Image segmentation data shows the porosity of the blood cake is 5.2%. FIG. 28C. SEM images of the blood cake post thermal lysis. Highest porosity is seen at 95° C. (66.8%). FIG. 28D. Bars graph of the dried blood cake porosity versus thermal lysis temperature (n=3 samples).

FIGS. 29A-29G. Characterization of biphasic LAMP reaction with and without thermal lysis, and biphasic LAMP reaction with pathogen lysis in whole blood. FIG. 29A. Simulation of porosity differences pre-(˜5%) and post-(˜67%) thermal lysis at 95° C. FIGS. 29B-29C. Single molecule detection MRSA and E. coli DNA in no thermal lysis control (low porosity) reactions from whole blood. Amplification threshold timings for detecting (FIG. 29B) MRSA and (FIG. 29C) E. coli. FIGS. 29D-29E. Amplification threshold times for (FIG. 29D) MRSA and (FIG. 29E) E. coli DNA detection in the biphasic reaction (high porosity reactions). For 1 copy amplifications, an expected 3 out of 8 amplifications are seen within 60 min of reaction time, due to Poisson sampling statistics. FIGS. 29F-29G. Characterization of pathogen lysis in whole blood. Amplification threshold times of biphasic reactions with (FIG. 29F) MRSA and (FIG. 29G) E. coli pathogens in 4 μL of whole blood. The bar graphs show mean and standard deviation data from 8 replicates of amplification. FIGS. 30A-30E. Biphasic reaction coupled with mechanical pathogen lysis by bead beating for detection limit of ˜1 cfu/mL for MRSA, MSSA, E. coli and Candida Albicans. FIG. 30A. Process flow schematic consisting of RBC lysis, mechanical bead lysis, drying, and biphasic reaction from whole blood. FIGS. 30B-30E. Amplification threshold data for the detection of (FIG. 30B) MRSA, (FIG. 30C) MSSA, (FIG. 30D) E. coli and (FIG. 30E) C. albicans pathogens in 800 μL of whole blood (8 curves for the 8 tubes per 800 μl starting blood sample). If not all 8 tubes amplified for a sample, the number of tubes that amplified is indicated above. 1 bar=1 sample of 800 μL of whole blood spiked with a specific cfu count (1e4 to 1 or 0).

FIGS. 31A-31D. Evaluation of the Biphasic approach using clinical sample. FIG. 31A. Threshold times of biphasic reactions for 14 amplified positive samples (13 E. coli and 1 MSSA) and average time (42.5±10.1 min, with green bar) and not amplified samples for negative samples. FIG. 31B. Table summarizing sensitivity and specificity of the biphasic approach against blood culture and identification using PCR. FIG. 31C. Overall time to result comparison between the biphasic and the blood culture+identification, and (FIG. 31D) Species-specific time to result comparison between biphasic (circle) and blood culture+identification (square) for E. coli (blue) and MSSA (red). Statistical comparison was performed.

FIGS. 32A-32B. SEM images of blood cake. Images of blood cake at different areas and magnifications of the blood cake at the bottom of tube pre (FIG. 32A) and post (FIG. 32B) thermal lysis at 95° C. Increased porosity of the blood cake post thermal lysis is clearly visible.

FIGS. 33A-33E. SEM images of blood cake post thermal lysis for increased time.

FIGS. 33A-33D. Images of the blood cake post thermal lysis at 95° C. for 5 (FIG. 33A), 10 (FIG. 33B), 15 (FIG. 33C), and 20 (FIG. 33D) minutes. Porosity level of blood cake does not increase with higher amounts of time for thermal lysis. FIG. 33E. Dried blood cake porosity versus thermal lysis time. The bar graphs show mean and standard deviation (n=3 samples).

FIGS. 34A-34D. Simulation and experimental characterization shows Biphasic LAMP reactions begin in the blood matrix. FIG. 34A. Simulation of BST polymerase reaching target DNA in blood matrix. Initially, at 500 um distance from DNA, the enzyme diffuses into the blood matrix. DNA does not diffuse out of the blood matrix. The enzyme reaches the DNA at around 30 min, when the amplification reaction starts. At 45 minutes the reaction is complete. FIG. 34B. Concentration as a function of time using the empirical rate equation for 1 copy (and 10000 copies as control) of DNA as initial concentration. ‘d’ signifies the distance of the single copy of DNA from the initial location of the enzyme (10000 copies of DNA are uniformly distributed over the blood matrix). Experimental values were averaged and normalized for comparison with simulations. FIG. 34C. Experimental characterization of biphasic reaction mechanism showing raw fluorescence data for the blood matrix only with 1e5 to 1 copy of MRSA DNA in 4 uL of whole blood. The supernatant was discarded post thermal lysis and reaction was done to detect DNA in the blood matrix only. Amplification threshold times from 8 replicates of amplification curves seen in FIG. 34D. The bar graph shows mean and standard deviation. 10 and 1 copy amplifications occur in the blood matrix showing DNA did not come out in supernatant. Also, it shows that enzymes and primers are able to reach the target DNA.

FIG. 35. Comparison of theoretical and empirical rates for simulated amplifications. Comparison of times of amplification for theoretical and empirical rates as a function of initial distance (t=0) of DNA from enzyme (1 copy of DNA as initial concentration). Trend lines match the rates very well in both cases.

FIGS. 36A-36D. Detection of cell free DNA using mec A resistance gene of MRSA and mal B gene of E. coli (O157:H7 serotype). FIGS. 36A-36B. Biphasic reaction without thermal lysis. Raw fluorescence data (for 8 replicates of data) for (FIG. 36A) MRSA and (FIG. 36B) E. coli DNA detection without thermal lysis in the biphasic reaction (reduced porosity reactions). FIGS. 36C-36D. Single molecule detection of MRSA and E. coli DNA. Raw fluorescence data (for 8 replicates of data) for detecting (FIG. 36C) MRSA and (FIG. 36D) E. coli DNA with 1 copy/4 μL limit of detection. For 1 copy amplifications, an expected 3 out of 8 amplifications are seen within 60 min of reaction time, due to Poisson sampling statistics.

FIGS. 37A-37D. SEM analysis of blood matrix porosity based on different drying conditions. FIG. 37A. SEM images of the blood cake dried on a hot plate set at 37° C. Image segmentation data shows the porosity of the blood cake is 15.5% pre thermal lysis and 60.9% post thermal lysis. FIG. 37B. SEM images of the blood cake dried on a hot plate at 50° C. Image segmentation data shows the porosity of the blood cake is 10.7% pre thermal lysis and 64.6% post thermal lysis. FIG. 37C. SEM images of the blood cake dried using dried on hot plate set at 95° C. Image segmentation data shows the porosity of the blood cake is 6.4% pre thermal lysis and 7.1% post thermal lysis. FIG. 37D. Dried blood cake porosity given different blood drying temperatures. The bar graphs show mean and standard deviation (n=3 samples).

FIGS. 38A-38F. Biphasic reactions with blood dried on hotplate at different temperatures. FIGS. 38A-38D. Raw fluorescence data and amplification threshold times (for 8 replicates of data) of biphasic reactions with MRSA DNA spiked blood dried on a hotplate at 37° C. (FIGS. 38A-38B) and 50° C. (FIGS. 38C-38D). Detection limit in both cases is 1 copy in 4 uL whole blood. FIGS. 38E-38F. Raw fluorescence data and amplification threshold times (for 8 replicates of data) of biphasic reactions with MRSA DNA spiked blood dried at 95° C. on a hotplate. Detection limit is 100 copies in 4 uL whole blood.

FIGS. 39A-39F. Sensitivity comparison to standard methods. FIG. 39A. Schematic of an analogous standard mixed reaction protocol from whole blood using LAMP reagents. Blood with DNA is directly mixed with LAMP reagents and reaction is conducted at 65° C. FIGS. 39B-39C. Raw fluorescence and amplification threshold time data (for 8 replicates of data) for mixed reactions with whole blood spiked with MRSA DNA template. The bar graph shows mean and standard deviation. The detection limit of mixed reactions is 1000 copies/4 uL of whole blood. FIG. 39D. Schematic of mixed reaction done on supernatant of centrifuged blood. Large volume of blood with spiked DNA was centrifuged to fractionate the blood. Supernatant with DNA was extracted into tubes, which was used to do the final mixed reaction. FIGS. 39E-39F. Raw fluorescence and amplification threshold data (for 8 replicates of data) for reactions with centrifuged blood with MRSA DNA template. The bar graph shows mean and standard deviation. The detection limit is 1000 copies/4 uL of supernatant.

FIGS. 40A-40B. Mixed reactions with E coli DNA in 4 uL of whole blood. Raw fluorescence and amplification threshold data (8 replicates of data) for mixed reactions with whole blood spiked with E. coli DNA template. Thermal lysis was not conducted in this reaction. The bar graph shows mean and standard deviation. The detection limit of mixed reactions is 1000 copies/4 uL of whole blood.

FIGS. 41A-41F. Characterization of pathogen lysis in PBS buffer and whole blood. Raw fluorescence data and amplification threshold times (for 8 replicates of data) of mixed buffer only reactions with (FIGS. 41A-41B) MRSA and (FIGS. 41C-41D) E. coli pathogens spiked in PBS buffer. Raw fluorescence data (for 8 replicates of data) of biphasic reactions with (FIG. 41E) MRSA and (FIG. 41F) E. coli pathogens in 4 μL whole blood.

FIGS. 42A-42B. SEM analysis of bead-lysed blood matrix pre and post thermal lysis. FIG. 42A. SEM images of the dried blood lysate post bead lysis. The bead beating blood lysate was dried on a hot plate at 95° C. Image segmentation data shows the porosity of the matrix is 11.3% pre thermal lysis and 66.2% post thermal lysis. FIG. 42B. Bar graph of dried blood lysate porosity pre and post thermal lysis. The bar graphs show mean and standard deviation (n=3 samples).

FIGS. 43A-43C. Biphasic reaction coupled with mechanical pathogen lysis by bead beating for detection limit of ˜-1 cfu/ml for MRSA, MSSA and E. coli. Raw fluorescence data for the detection limit of (FIG. 43A) MRSA, (FIG. 43B) MSSA and (FIG. 43C) E. coli pathogens, which is 1 cfu per 800 μL of whole blood. Each plot demonstrates a different set of experiments, each containing a concentration of 104-0 cfu/mL.

FIGS. 44A-44B. Biphasic reaction coupled with mechanical pathogen lysis by bead beating for specificity tests for MRSA and MSSA primers. Raw fluorescence data and threshold time for the detection of (FIG. 44A) MRSA pathogen using MSSA primer and (FIG. 44B) MSSA pathogen using MRSA primer. MSSA primer could detect the presence of MRSA pathogen. On the other hand, MRSA primer which detects the mec A gene couldn't detect the presence of MSSA primer.

FIGS. 45A-45B. Protocols for the control experiments with optical images. FIG. 45A. A biphasic protocol using 0.8 mL volumes used for measurement of control experiments, mock samples, and patient samples. Mechanical bead lysis induces the bacterial cell lysis, giving reagents access to DNA and achieving improved sensitivity for pathogen detection. After vortex, blood lysate post bead-based mechanical pathogen lysis is transferred into 8 tubes (1 PCR strip, 30 μL each). Then, either biphasic protocol or mixed reaction was performed and compared. FIG. 45B. Comparison of the protocols including biphasic, mixed, only thermal lysis and only dry reactions. The optical images show distinct contrast between the clear supernatant of biphasic reaction and the turbid color of the mixed reaction.

FIGS. 46A-46E. Control experiments data with lysate post blood processing and bead beating of E. coli pathogen spiked in 800 μL whole blood. FIG. 46A. Comparison between biphasic reactions and mixed reactions for 104-10² cfu/mL concentration. Comparing the fluorescence intensity at the time the reaction is finished (at 55 min), the biphasic reaction (˜60,000) shows about 10 times higher fluorescence than the mixed reaction (˜6,000), representing the advantage of liquid phase through clear supernatant.

FIGS. 46B-46C. Raw fluorescence and amplification threshold data for (FIG. 46B) mixed reactions and (FIG. 46C) only thermal lysis reactions with E. coli pathogen post processing of 800 μL of blood through RBC lysis and mechanical bead beating. The detection limit of mixed reactions is worse than 1e2 cfu/800 μL of whole blood for both mixed reaction and only thermal lysis reactions. FIGS. 46D-46E. Raw fluorescence and amplification threshold data for only dry reaction with (FIG. 46D) MRSA and (FIG. 46E) E. coli pathogen post processing of 800 μL of whole blood. In case of 1-10 cfu/mL concentrations, 4 replicates were tested. The bar graph shows mean and standard deviation. The number on the bar means the number of amplified tubes out of 8 tubes (1 strip, 30 μL each). If all 8 tubes were amplified, the numbers were not marked on the bars.

FIGS. 47A-47B. Analysis of heme content in biphasic and mixed reactions with whole blood and bead lysed lysate matrix. FIG. 47A. Brightfield images were taken of the reaction mix of biphasic and mixed reactions of whole blood and bead lysed blood lysate shown in the tube images. The red channel component (due to heme content in blood) of each image was extracted. Red areas, shown as white for high concentrations of heme content, was quantified through image segmentation. FIG. 47B. Bar graph showing percentage of red area in each segmented image in panel A. The bar graphs show mean and standard deviation (n=3 samples).

FIGS. 48A-48F. Reaction optimizations for 800 uL Blood spiked with C. albicans and processed with our platform. Raw fluorescence and amplification threshold data for biphasic reactions for C. albicans fungal pathogens. 800 uL of whole blood with pathogens was processed through blood lysis and bead beating for pathogen lysis as mentioned in the methods section. Final lysate from a single 800 ul blood sample was distributed into 8 tubes (30 ul lysate per tube) and dried for downstream biphasic reaction. FIGS. 48A-48B. The amplification reaction was done at 62° C. with 0.15 μM of F3 and B3, 1.17 μM of FIP and BIP, and 0.59 μM of LF and LB primers. FIGS. 48C-48D. The amplification reaction was done at 67° C. with same primer concentrations as above. FIGS. 48E-48F. The amplification reaction was done at 67° C. with 0.04 μM of F3 and B3, 0.33 μM of FIP and BIP, and 0.17 μM of LF and LB primers (reduced primer concentration). All concentrations mentioned are the final concentrations in 96 uL of final reaction. Bar graphs show mean and standard deviation of threshold times of amplification for each sample (8 curves for the 8 tubes per 800 ul starting blood sample. If not all 8 tubes amplified for a sample, the threshold time average and the number of tubes that amplified is indicated above the data of that sample). 1 bar graph is 1 sample of 800 ul of whole blood spiked with a specific cfu count (1e4 to 1 or 0) in the above figure.

FIGS. 49A-49B. Biphasic reaction coupled with mechanical pathogen lysis by bead beating for detection limit of ˜1 cfu/ml for Candida Albicans with optimized reaction parameters. FIG. 49A. Raw fluorescence data for biphasic reactions for C. albicans fungal pathogens in the range from 1e4 to 0 cfu/800 μL whole blood. 800 μL of blood spiked with pathogens was processed through blood lysis and bead beating for pathogen lysis as mentioned in the methods section. Final lysate from a single 800 μL blood sample was distributed into 8 tubes (30 μL lysate per tube) and dried for downstream biphasic reaction. 1 bar graph is 1 sample of 800 ul of whole blood spiked with a specific cfu count (1e4 to 1 or 0) in the above figure and show LOD of 1.2 cfu/ml. FIG. 49B. Raw fluorescence data for 800 uL of whole blood spiked with 10 and 1 cfu per sample of C. albicans pathogen with 6 replicates, respectively. All bar graphs show mean and standard deviation of threshold times of amplification for each sample. The amplification reaction in this figure was done with optimized parameters, i.e. at 67° C. with 0.04 μM of F3 and B3, 0.33 μM of FIP and BIP, and 0.17 μM of LF and LB primers. All concentrations mentioned are the final reaction concentrations in 96 μL of reaction mix. (8 curves for the 8 tubes per 800 μL starting blood sample. If not all 8 tubes amplified for a sample, the threshold time average and the number of tubes that amplified is indicated above the data of that sample).

FIG. 50. Clinical identification and Biphasic results for whole blood samples from patients in the ED that have a blood culture ordered. 63 clinical samples (15 negative and 48 positive) were tested. Total time for clinical lab results was calculated by adding the fixed identification time (3 hrs) to time to positive culture. Total time for our process was calculated by adding the fixed sample preparation time (1.5 hrs) to threshold time. Sample 46, 47, 48, 52 and 53 were examined against more than one primer for specificity tests in our process.

FIG. 51. Comparison of the time between time to positive culture and amplification time.

FIGS. 52A-52B. Nucleic acid amplification processes and inhibition mechanisms.

FIG. 52A. Nucleic acid amplification and (FIG. 52B) Inhibition mechanism by nonspecific binding of various inhibitor components found in blood.

FIGS. 53A-53D. Biphasic reaction for highly sensitive nucleic acid amplification.

FIG. 53A. Current state-of-art techniques involve extraction and purification with polymerase chain reaction. Target DNA is lost due to inherent loss mechanism during the binding and elution of the DNA. FIG. 53B. General trend of recovery rate versus extraction and purification steps in FIG. 53A. FIG. 53C. Proposed biphasic method uses two heating steps to inactivate inhibitors while retaining the target DNA within the dried blood matrix.

FIG. 53D. There is no loss of target DNA during the process because relatively high inhibitor level does not affect the amplification since they exist in an inactivated form. As a result, recovery rate remains consistently high regardless of initial total DNA amount.

FIGS. 54A-54E. Inhibitor characterization using colorimetric (for hemoglobin) and ELISA (for IgG) measurement. FIG. 54A. Images of blood samples with various conditions along with blood drying process and after further incubation. Inhibitor concentration for (FIG. 54B) hemoglobin and (FIG. 54D) IgG, before and after drying. Further characterization of inhibitor level for (FIG. 54C) hemoglobin and (FIG. 54E) IgG, using various treatment to see if physically locked inhibitors are released out to the supernatant after incubation.

FIGS. 55A-55B. LESA-MS workflow and results for the analysis of inhibitor levels either inside of or released from the dried blood matrix. Protocols of the pre-processing for different types of samples: (FIG. 55A) dried blood matrix and supernatant incubated with different conditions. FIG. 55B. Inhibitor levels for heme B, α-globin and β-globin measured by LESA-MS with various conditions: nuclease free water, supernatant, and dried blood matrix. Major peaks corresponding to heme B, α-globin and β-globin in different charge states were labeled. Peak abundance was normalized to heme B intensity in dried blood matrix.

FIGS. 56A-56D. Characterization of biphasic assay sensitivity with various conditions (drying time, sample volume and material properties). FIG. 56A. Longer drying time results in increased target concentration (target amount/unit volume) and increased calculated probability of amplification. FIGS. 56B-56C. Sample volume affects the diffusion distance and hence the probability of amplification. LAMP characterization with the comparison between two sample groups: 30 μL in a one tube versus three tubes with 10 μL each using (FIG. 56B) E. coli DNA and (FIG. 56C) MRSA DNA spiked in whole blood. FIG. 56D. Effects of material properties on the detection sensitivity using whole blood (WB) and blood lysate (BL).

FIGS. 57A-57E. Application of blood drying and biphasic reaction using recombinase polymerase amplification (RPA). FIG. 57A. Schematic workflow of RPA for biphasic reaction. FIG. 57B. Fluorescence detection method using the RPA Exo probe. FIGS. 57C-57E. Limit of detection characterization using different DNA concentration and pathogen concentration. FIG. 57C. Effects of the heating step 2 for achieving a higher sensitivity. FIG. 57D. Fluorescence level comparison between biphasic and direct reaction using 103 and 102 copies/μL. 10 times higher signal to noise ratio was obtained. FIG. 57E. Assay characterization using pathogen spiked in whole blood.

FIGS. 58A-58G. Blood drying characterization with various conditions such as drying temperature, drying time, different volumes, and materials. FIG. 58A. Images of dried blood matrix in PCR tubes using three volumes (4, 30, 100 μL) at different drying temperature. Pipet tips were used to verify if the blood was fully dried by checking for any liquid blood that could be extracted. The absence of blood in the pipet tips indicated that the samples were completely dried. FIG. 58B. The duration of time required to fully dry a specific volume of whole blood. FIG. 58C. Pictures of the dried blood matrix from a 30 μL sample of whole blood, taken after 20 minutes of drying, show that the blood components became detached from the PCR tubes and were suspended in the buffer mix upon its addition. FIG. 58D. Images of 10 (upper 4 PCR tubes) and 30 μL (lower 4 PCR tubes) sample ready for biphasic reaction. FIG. 58E. Weight loss (%) of the 10 μL whole blood according to drying time. Difference in (FIG. 58F) Density and (FIG. 58G) weight loss when comparing the whole blood (WB) and blood lysate (LB).

FIG. 59. Characterization of diffusion distance for reagents within the dried blood matrix in PCR tubes with volumes of 4, 10, and 30 μL of blood. Diffusion distance is highly important in biphasic reaction, and it can be simplified to the height of the dried blood matrix in PCR tubes. Specifically, the height of blood in tubes containing 4 μL was between 1000 to 2000 μm, while those containing 10 μL showed a diffusion distance of around 2000 μm. In contrast, tubes containing 30 μL blood required a diffusion distance greater than 5000 μm. Based on our previous simulation data, which showed the diffusion of Bst polymerase (with a diffusion constant at 65° C. of 5.63×10⁻¹¹ m²/s) in blood matrix to reach the target DNA (1 copy), a diffusion time of 35 to 50 minutes was required to initiate the amplification for a diffusion distance of 500 to 1000 μm. Therefore, the diffusion distance for the 30 μL volume of blood in the PCR tube may be too long for the polymerase to reach the target DNA within the reaction time (60 minutes), especially considering the distance is farther than 5000 μm.

FIGS. 60A-60E. The shape of the whole blood (WB) and blood lysate (BL) in the PCR tubes when they are dried. FIG. 60A. Once the 30 μL WB and BL in the tube were dried, the bottom of the tubes were cut and (FIG. 60B) recorded with image from the top and bottom view. WB demonstrated no hollow space at the center while BL showed a big hall. FIG. 60C. Afterward, the cut tubes were located above the new PCR tubes and water was added to the upper tube to see if there is any drainage. As a result, only cut tube with BL demonstrated drainage of water so that water is located at the bottom tube. On the other hand, water added to the cut tube containing dried WB stayed above the dried blood matrix of WB, implying minimum spread of liquid in macro scale. FIGS. 60D-60E. Physical difference of WB and BL when they are dried. 100 μL of WB and BL were spotted on Petri dish and dried at 95° C. for 10 minutes. FIG. 60D. WB showed significant compaction toward the center of the liquid so that sharp structure looking like a triangle can be found. FIG. 60E. In contrast, BL retained their shape once they dried. This compaction can explain the reduced coffee ring effect in WB and consequent low sensitivity compared to the BL.

FIG. 61 illustrates a configuration of positioning electrodes across a unit cell of dried blood (e.g., a dried blood sample island) having liquid supernatant with liquid capable of transiting the network within the dried blood sample islands. The bottom and top electrodes provide the ability to tailor maximum electric field intensity to dried blood sample islands containing the introduced liquid (labeled as buffer with reagents).

FIG. 62 illustrates a substrate formed from a Si wafer (left panel) having a plurality of microwells. The middle panel is a top view of an SEM of the Si wafer with attendant dimensions. The right panel illustrates the pixelated configuration, where whole blood is dried into a plurality of dried sample islands (hence “pixel”), wherein the well associated with each island can hold a volume of about 100 μL, and readily about 5 mL of whole blood total on the wafer, for a total of about 50 individual blood pixels. In this manner, the platform is readily scalable and multiplexible for any of a number of different targets.

FIG. 63A-63D illustrates a bi-phasic reaction on a micro-well array. FIG. 63A illustrates the single tube configuration can be replaced with an array, including for use of large whole blood sample volumes (5 mL of blood per wafer substrate). FIG. 63B is an optical image of a 5625 microwells array next to a quarter to illustrate scale. FIG. 63C is an SEM image of microwells. FIG. 63D show the microwells array post-blood loading.

FIG. 64 schematically illustrates bacteria that is ruptured by an electric field positioned within the dried blood matrix. Dried blood islands with the network filled with an ionic fluid act as a local amplifier of the electric field in locations where the blood-borne pathogen (such as a bacteria) is likely located.

FIG. 65 schematically illustrates other electrode configurations for a microwell array having microfluidic and electrical lysis capabilities. Each unit cell of the microarray is illustrated configured to contain 100 μL of blood (600 μm×600 μm×300 μm) separated from adjacent wells by about 20 μm. In this manner, the whole blood is pixelated into small sample sizes and dried so that there are a plurality of dried blood islands ready for a biphasic reaction.

### DETAILED DESCRIPTION OF THE INVENTION

As used herein, “substrate” refers to a material, layer or other structure having a surface, such as a receiving surface, supporting one or more components or devices including a microarray. Arrays may be embedded in substrates so that the array is formed within and made with the same material as the substrate. Arrays embedded in substrate may be manufactured from a single piece of material. In some embodiments, the array is a microarray. Substrates which may be useful in the methods and devices described herein include silicon, glasses, metals, insulators and/or dielectrics. Substrates may be composite materials. The substrate and/or supported microarray may be referred to as a chip herein.

“Array” refers to material or device having a number of wells, receiving chambers, void spaces or is otherwise configured to hold a number of liquid tissue samples. Arrays may have any number of wells and may be provided in various configurations including a grid, as described herein. Wells useful in the described arrays may have any geometric shape including pyramids, cones, and rounded bottom wells with circular, square or polygonal cross-sections. Arrays may include wells having more than one dimension (e.g. depth, width), volume and/or shape. Arrays may have greater than or equal to 10,000 individual wells, greater than or equal to 100,000 wells, or optionally, greater than or equal to 1,000,000 wells. A “microarray” refers to an array of wells where at least one dimension is less than 1 mm.

“Target analyte” refers to a molecule that is desired to be detected. An example of a target analyte is a molecule that is capable of being amplified, such as a nucleic acid (DNA or RNA), so that a single target analyte may be exponentially amplified by a technique such as PCR or an isothermal technique such as LAMP.

“Liquid sample” is used broadly to refer to any sample that is capable of flowing under applied shear. Accordingly, the sample may originally be a non-fluid, such as a tissue or food, but that is suspended in a fluid solvent material, so that the original solid sample is a liquid sample. Alternatively, the sample may originate as a generally liquid sample. The sample may be a “biological sample” from a human, animal, a tissue, or a cell line. A particularly preferred liquid sample is a blood sample, including a minimally processed blood sample. “Minimally processed” refers to the obtained liquid sample where no undue processing, purification, preservation has occurred. The methods and devices, of course, are compatible with processing, including a minimally processed harvest such as application of an anti-coagulant or fluid to achieve a desired fluid parameter (e.g., viscosity) to facilitate fluid spreading over the array. “Unprocessed” refers to direct application of a fluid sample to the array, without intervening processing steps.

“Dried sample islands” refers to those portions of the originally-applied liquid sample that are positioned in wells and are capable of being contacted with relevant liquid-suspended reagents, for potential amplification of one or more target analytes, if present. The dried sample islands have a fluidic network. “Fluidic network” is used broadly to refer to voids, passages, or the like, within the dried sample island, including the dried blood sample island, that occurs when the liquid sample is dried on the substrate. The fluidic network may comprise microchannels, nanochannels and/or pores, so that fluid from outside the dried sample island can access the interior of the dried sample island and nucleic acid, including amplicons, may diffuse from the interior of the blood sample island to liquid supernatant overlaying the dried blood sample island.

“Bi-phasic reaction” refers to a reaction that occurs in each of two phases, such as a liquid phase and a solid or semi-solid phase over which the liquid phase is suspended. In the context of the instant technology, the dried sample in the well may contain target analyte of interest, and the liquid phase the necessary components for amplification of the target analyte. Target analyte may diffuse or be otherwise conveyed into the liquid phase for corresponding reaction. Similarly, the necessary components from the liquid phase (referred herein generally as “reagent(s)”) may diffuse or otherwise mix into the solid dried phase, with corresponding reaction. For example, the liquid with reagents may penetrate into an interior portion of a dried sample by passive diffusion and/or be guided by a network of passages or pores formed in the dried sample, with the reaction then considered within the dried sample, particularly for passages or pores that are micro (up to 1 mm) or nano (up to 1 μm) sized. Conceptually, then, the amplification may be considered to arise from each of the “original” liquid and dried solid phases so that the amplification is generally characterized as a “biphasic reaction”.

“Pathogen” refers to any material in a blood sample indicative of a material that could be of concern to a patient. Examples include, but are not limited to a bacteria (including an antibiotic resistant bacteria, an antibiotic-receptive bacteria, a gram-positive bacteria, a gram-negative bacteria or any other bacteria of interest, including for sepsis-related application), a fungus, a virus, a parasite and any free-floating nucleic acid in the blood. More broadly, a pathogen could also reflect a disease state, such as a cancer condition or any cell of interest. Such disease state material may be any cell in the blood having a marker indicative of a disease, such as a cancer cell or genetic material related thereto.

“High surface area structures” refers to structures that are specially configured to present increased surface area to ensure appropriate reactions. The term is used broadly, and encompasses bubbles in the solid that readily increase surface area of the dried sample available to reagent or any other process that results in passages or pores in the structure. Similarly, the reagent liquid may contain the high surface area structure, including beads to which one or more reagents are connected. The beads may then be used as a means to ensure intimate contact between reagents and target analyte, including in the solid phase.

“Reagent connected bead” refers to a reagent that is connected to the bead. The connection may be covalent or non-covalent. The connection may be via one or more linkers or receptors on the surface of the bead having specificity for the reagent.

“Microrelief structure” refers to a high surface area structure with one surface having a plurality of features that extend in a direction away from a planar surface. This effectively increases the surface area of the structure, and specifically is used to accommodate attachment of molecules, thereby increasing the number of molecules while maintaining good accessibility to liquid phase in the wells. The relief structure that is a microrelief structure refers to at least one dimension of the relief structure or spacing that is less than 1 mm. This particularly includes the thickness of the relief structure.

Incorporated by reference herein is U.S. Pat. No. 11,732,293 titled “Biomarker Detection from Fluid Samples” (Atty Ref. 338305: 91-17 US), which is specifically incorporated by reference herein for the devices and methods described therein.

### Example 1: Dried Liquid Sample (Blood, Urine, Saliva, Etc.) Digital Amplification

Described herein is dried sample digital amplification (LAMP, PCR etc.) on a substrate (Glass, plastic, silicon etc.) with an array of microwells and with no/minimal sample processing. The drying of the sample allows processing of larger sample volumes (up to several milliliters of blood for e.g.) and preservation of pathogen nucleic acids in the sample for room temperature transportation and storage. Additional fixation of samples can be performed of the samples using common fixatives such as Formalin or acetone for extended storage of the samples for analysis.

Digital amplification (digital lamp, digital per etc.) is performed on the array of wells or microwells with dried sample in it. The array may have large number of wells (1e5-1e6) in line with large number of partitions required for digital amplification. This technique combines higher sample volume processing capability with superior sensitivity and limits of detection from digital amplifications down to single molecules starting from crude-non-purified samples. This allows the targeting of low abundance pathogens for example in sepsis, where the bacteria count can be as low 1-3 cfu/ml and unless a few ml of blood is processed, the pathogen may be excluded from the sample. Currently, no such technique exists in the literature and thus conditions such as sepsis requires culture of the pathogens before any amplification. Also, as with all digital reactions, the technique will be end-point, won't require any standard curve needed for qPCR etc., and give the exact count of pathogens at the end.

In conclusion, this technique provides a new simpler way of doing digital amplification on a petri-dish with microwells, process large sample volumes and without the usual sample purification steps. This will replace the culture method for sepsis and other conditions and make the purification steps before amplification obsolete.

To perform the reactions and amplify DNA/RNA from dried samples, high surface area structures may introduced or produced in the sample using microbubbles, microposts, etc. These high surface area structures allow easy accessibility of amplification enzymes to the pathogen even in the presence of other sample debris. High surface area structures formed in dried blood are described herein and shown in provided SEM images.

Detection modality: Optical—Using fluorescent probes or dyes (e.g., Sybrgreen). Electrical—Using pH change through Ion sensitive field effect transistors (ISFET) chips. Dried sample processing—Blood smear, urine, saliva, or a solid sample that has been liquefied.

Targeted Problem: Current primary healthcare screening and diagnosis still primarily relies on age-old techniques such as culture, microscopy, immunoassays, and on a physician's own experience. All these techniques are very time-consuming, qualitative and laborious, or lack the appropriate sensitivity and functionality to measure parameters such as drug resistance. Even after significant breakthroughs in sensitive and quantitative nucleic acid amplification testing in the past decade, information on panel of nucleic acids (mRNA biomarkers and pathogen nucleic acids) associated with a pathology (such as viral vs bacterial respiratory tract infection, their specific strains and drug resistance) is usually not available due to the limited capacity to multiplex and the associated costs and time. This has not only led to sub-optimal patient diagnosis and poor experience but also given rise to other problems such as drug resistance due to antibiotic overuse¹. We describe a minimal sample preparation, highly multiplexed (100 or more targets from single crude sample—blood, saliva etc.), sensitive, quantitative and low-cost nucleic acid amplification technique (NAAT) from dried crude sample using loop mediated isothermal amplification (LAMP) on commercially available ion-sensitive field effect transistors or using optical detection systems (for labs).

Significance: The translation of molecular testing into routine clinical practice has been hindered by several factors such as time, multiplexing capability and cost. The current protocol for molecular testing usually involves collecting sample from the patient (Blood/oral swab/vaginal swab/urine etc.), purifying the analytes (proteins or nucleic acids) and finally running immunoassay or NAATs^{2,3}. For instance, in cervical cancer screening, a pap smear microscopy and a HPV DNA test through PCR are both performed on separate samples (PCR done after analyte purification) to check for abnormal lesions and viral infection, respectively. These separate isolated tests usually take days to complete and the patient must visit the doctor's office multiple times until a diagnosis. Moreover, using the same sample, tests for other common sexually transmitted infections (STI) such as Chlamydia and Gonorrhea are usually not performed as it significantly adds to costs and time to multiplex higher number of targets in the current process flow. Rapid and cheap multiplexed tests can be extremely helpful for conditions such as chlamydia or gonorrhea, which are the most prevalent STIs worldwide, and for which up to 80% of the patients can be asymptomatic and undetected infections can lead to tubal infertility and other complicationss⁴. Among other frequently ordered tests at primary healthcare facilities, common infectious diseases form a major portion. For respiratory tract infections, it is often difficult for the clinician to distinguish between viral and bacterial etiologies, and this results in overuse of antibiotics^{1,5}. Conventional diagnosis using culture, antigen detection or serology is either too slow or too insensitive⁶ and although sensitive nucleic acid amplification methods exist, the cost to multiplex several targets prevents its use as the first choice for diagnosis⁵. This is yet another scenario where a rapid, cheap and highly multiplexed NAAT is extremely useful. For tuberculosis, where the majority of disease burden lies in low or middle income countries, the conventional diagnosis relies on sputum microscopy, solid culture and chest radiography, all of which lack in sensitivity and specificity. Recently developed immunoassays such as QuantiFERON®-TB perform satisfactorily but cannot detect multi-drug resistance or HIV-associated tuberculosis^{7,8}. Laboratory based sensitive nucleic acid amplification tests have been developed but few platforms (e.g. Cepheid® GeneXpert®, Roche® LightCycler® SeptiFast) provide point-of-care and rapid results⁷. This platform performs automated sample purification making it extremely expensive (GeneXpert® IV instrument-US$17000, individual cartridge ˜15 US$) and multiplexing cannot be performed on the same cartridge⁹. For developing countries where sophisticated automated cell counters are still rare, blood smear microscopy is the primary technique for complete blood count and diagnosis of blood related conditions such as Malaria and Anemia¹⁰. Cheap and rapid nucleic acid testing without any sample preparation and on the same smear substrate for possible demographic infections will allow termination of continuous residual transmission of many infectious diseases^{11,12}. For viral infections such as HIV, the point-of-care immunoassay based tests are nonquantitative and cannot be used for early diagnosis, antiretroviral therapy or infant diagnosis⁴.

All the issues discussed above call for a technique that demonstrates the following features: 1. no/minimal sample preparation—reactions directly from crude biological samples; 2. highly multiplexed detection of molecular markers and co-infections from a single sample—Complete pathological profile of the patient leading to data driven decision making; 3. sensitive, specific and quantitative; 4. low instrument cost and cost per test; 5. preferably without expensive and bulky optical detection systems which also limit the number of reactions (Field-of-view for imaging is usually small); 6. preferably without active pumps or similar flow systems which require high power and are one of the main causes of experiment failures.

Molecular testing on your USB stick: Loop-mediated isothermal amplification (LAMP), which has emerged within the last two decades as an alternative to PCR for nucleic acid amplification, has been leveraged for higher specificity of its 4-6 primers, single-temperature incubation (60° C.), single molecule sensitivity and increased resistance of the Bst polymerase to inhibitors that prevent PCR¹³. We and two other groups have demonstrated RT-LAMP reactions from whole blood and with excellent sensitivity down to 100 pfu/ml of viral targets such as Zika or HIV¹⁴⁻¹⁶. LAMP reactions with good sensitivities (50-100 pfu/ml) using minimally processed saliva and urine samples have also been reported^{14,17}.

The robustness of LAMP to biological contaminants in multiple unpurified sample types makes it suitable for use in our detection technique. However, the detection modality for all previous nucleic acid amplification techniques has been primarily optical making the instrument expensive and limiting the number of reactions that can be simultaneously performed (Limited field-of-view). Recently, Ion sensitive field effect transistors (ISFET) have been shown in literature for the direct detection of LAMP products through a pH based approach^{18,19}.

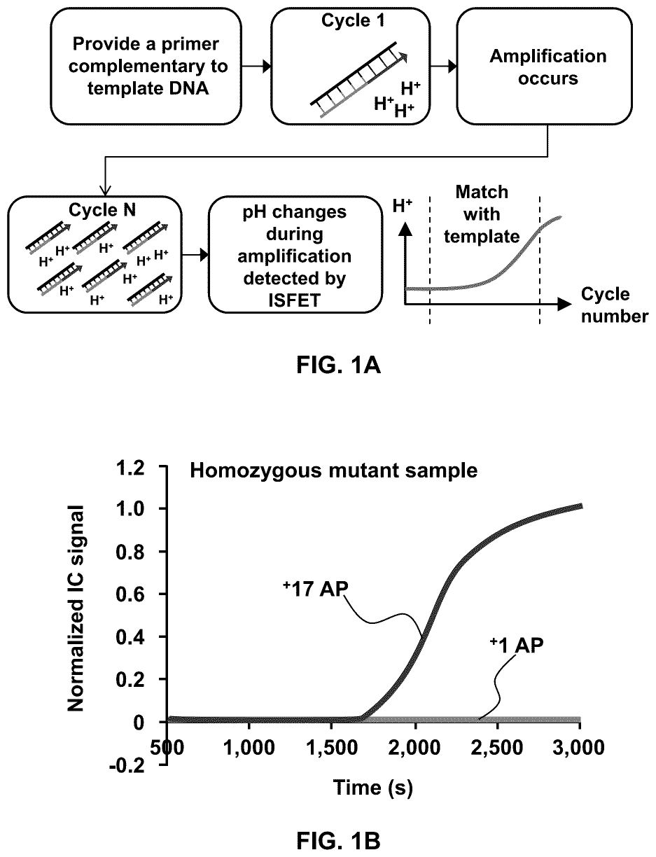

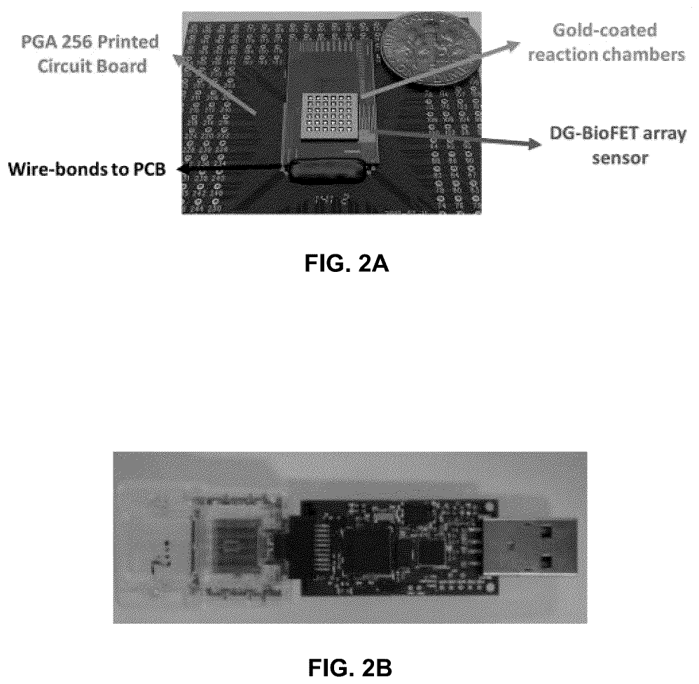

The incorporation of nucleotides into the elongating strand of nucleic acid in a LAMP reaction releases hydrogen ions which decrease the solution's pH. This drop in pH can be monitored electrically by the ISFETs (FIGS. 1A-1B). Our group has also recently demonstrated pH-LAMP reactions from purified DNA samples on a chip containing 1 million foundry-fabricated ISFETs (FIG. 2A and FIG. 2B)¹⁹. These devices use state-of-the-art CMOS fabrication techniques to produce a million sensors on a 7 mm*7 mm area. This greatly reduces the footprint of our device, and enables massively parallel reactions that can detect multiple targets at the same time in a single LAMP reaction (100-1000 ISFETs per reaction with 1 million ISFETs per chip=>1000 parallel reactions possible).

Apart from the massive targeted multiplexing, eliminating active microfluidic flow systems which require microfluidic pumps would not only help in bringing down the cost per test, but also simplify the process-flow and reduce the number of potential malfunctioning elements. Described herein is the testing from dried samples (blood from finger prick, saliva etc.) on wax paper or glass slide. Dried blood spots (DBS) have been used in NAATs for the direct detection of analytes with minimal sample purification²⁰. The stability of nucleic acids in dried samples has also been well characterized with HIV-1 RNA being shown to be stable in dried blood spot for a year at room temperature²¹.

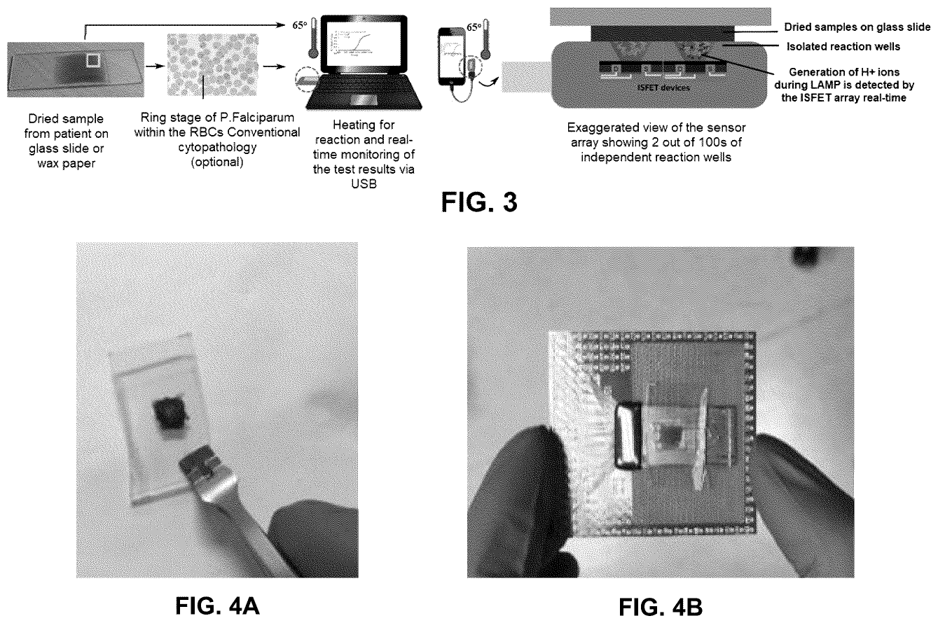

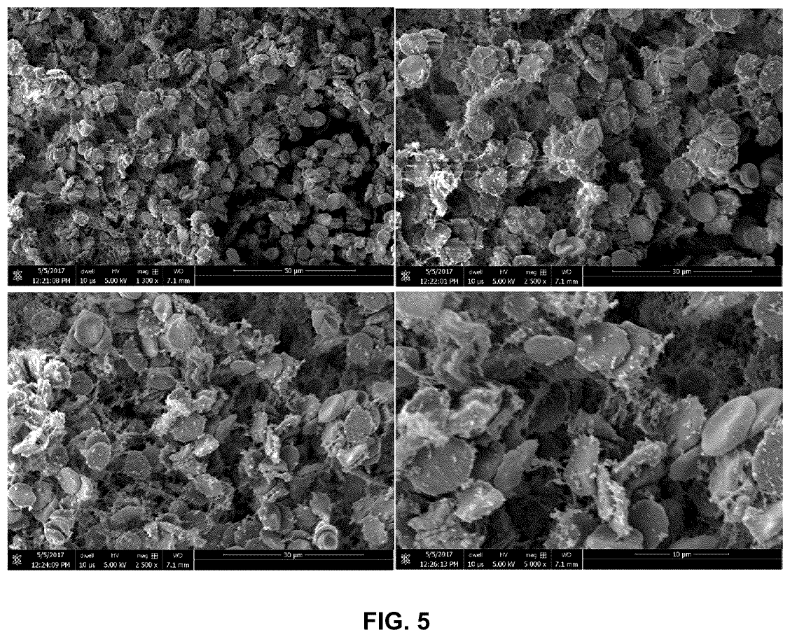

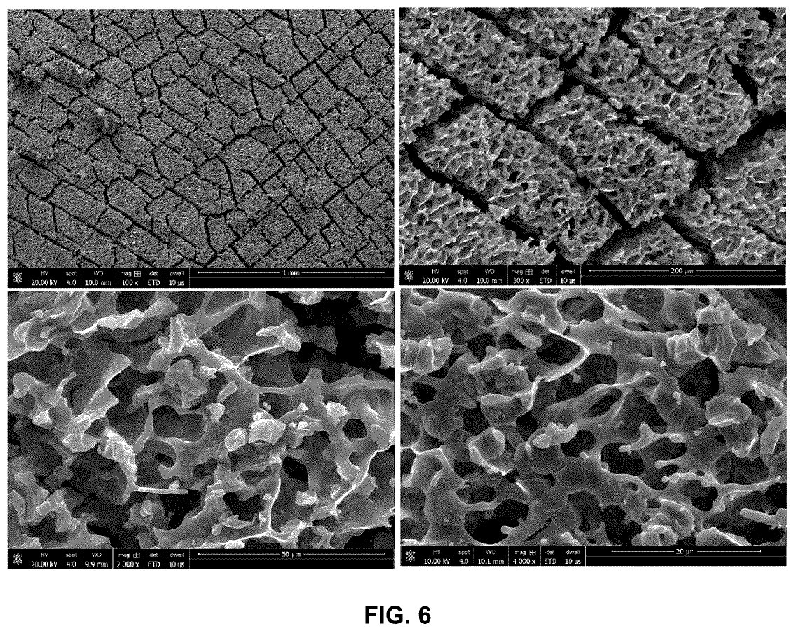

Described herein is the combination of pH-LAMP reactions on commercially available chips with the process of drying crude biological samples on a glass slide or wax paper with microwell array. These slides or wax papers are pre-printed with the primers of a panel of chosen nucleic acid biomarkers or pathogen sequences using cheap commercially available microarray printing technology²². FIG. 3 shows the schematic and the overall process flow for our technique. Briefly, dried biological samples (blood, saliva, urine etc.) on a glass slide/wax paper with microwells interface with our transistor chip. The template-free amplification reagents will be loaded into the wells from a single input and LAMP reaction will be performed at 65C. The dried template in the crude sample will rehydrate in the LAMP solution and initiate amplification reaction once a matching template-primer pair is found, and, the real-time change in pH during the reaction is monitored by the ISFET sensor surface.

The use of glass slide as an optional substrate also enables conventional cytopathology to be done on the same slide prior to the molecular testing in a primary healthcare facility. FIGS. 4A-4B show a sample chip bonded to a glass slide containing dried blood for pH-lamp measurement. Electrical detection of LAMP reaction will significantly reduce the footprint of the system and enable the sensing system to be coupled to a USB stick (FIG. 3). This allows end users to use the 5V output from laptop or smartphones to perform the isothermal amplification reaction and immediately view the results on their devices.

Impact: To summarize, described is a technique that follows through on all the parameters discussed in significance—No sample preparation and rapid, highly multiplexed, sensitive, quantitative and very low-cost. As the trends are shifting towards molecular diagnostics, the ability to visualize a panel of 100-1000 or more nucleic acid biomarkers and pathogens from the same starting sample within 30 minutes will cause a paradigm shift towards data-centered diagnostics and decision making. With our commercially available ISFET devices from Taiwan semiconductor manufacturing company (TSMC), major chip fabrication players (INTEL or TSMC) can be an active part of the product manufacturing pipeline, significantly reducing the time-to-market and costs (for reference, a 4 GB USB with billions of transistor costs <2$ off the shelf²³⁻²⁵), and bringing their expertise in electronic device fabrication.

For home based diagnostics or surveillance, the product market is currently restricted only to a few basic devices since the 1970s, such as glucose monitoring for diabetic patients. There are no at-home tests for cancer molecular screening, common infections or for recurring measurements in response to treatment such as for HIV patients. As we move towards quantitative data driven medical science and treatment decisions, our one-step, rapid, cheap, massively multiplexed and simple detection of molecular biomarkers or pathogens will not only impact primary healthcare facilities but also cater to the huge market of home-based molecular diagnostics.

Higher blood processing through blood drying: Diseases such as sepsis where the pathogen count is a few cfu/ml require high blood volume processing capabilities to detect the pathogen of interest. The current mode of diagnosis is blood culture which takes 3-5 days and the chances of mortality during this time are extremely high. The conventional purification steps with commercial RNA/DNA extraction kits are not efficient in extracting rare pathogen DNA in the presence of significantly more abundant human nucleic acids. We process dry blood on chip, pixelate it and perform digital PCR or LAMP directly from the pixelated dried blood.

Use of microbubbles to create a high surface area structures: Microbubbles filled with gas and mixed with whole blood will create higher surface area microstructures within the dried blood and expose the bacteria, allowing easy access of enzymes to the pathogen for amplification.

### REFERENCES

1. Lundborg, C., Mölstad, S. & Olsson, E. Antibiotic prescribing in outpatients: a 1-week diagnosis-prescribing study in 5 counties in Sweden. Scand. J (2002).
2. Akhmetov, I. & Bubnov, R. V. Assessing value of innovative molecular diagnostic tests in the concept of predictive, preventive, and personalized medicine. EPMA J 6, 19 (2015).
3. Fauci, A. S. & Morens, D. M. The Perpetual Challenge of Infectious Diseases. N. Engl. J Med. 366, 454-461 (2012).
4. Mabey, D., Peeling, R. W., Ustianowski, A. & Perkins, M. D. Diagnostics for the developing world. Nat. Rev. Microbiol. 2, 231-240 (2004).
5. Brittain-Long, R. et al. Multiplex real-time PCR for detection of respiratory tract infections. J. Clin. Virol. 41, 53-56 (2008).
6. Gunson, R., Collins, T. & Carman, W. Real-time RT-PCR detection of 12 respiratory viral infections in four triplex reactions. J. Clin. Virol. (2005).
7. Lawn, S. D. et al. Advances in tuberculosis diagnostics: The Xpert MTB/RIF assay and future prospects for a point-of-care test. Lancet Infect. Dis. 13, 349-361 (2013).
8. McNerney, R. & Daley, P. Towards a point-of-care test for active tuberculosis: obstacles and opportunities. Nat. Rev. Microbiol. 9, 204-213 (2011).
9. Cepheid|GeneXpert IV. Available at: http://www.cepheid.com/us/cepheid-solutions/systems/genexpertsystems/genexpert-iv. (Accessed: 30 Apr. 2017)
10. Bain, B. J. Diagnosis from the Blood Smear. N. Engl. J Med. 353, 498-507 (2005).
11. WHO|A WHO external quality assurance scheme for malaria nucleic acid amplification testing. Meeting report. WHO (2016).
12. WHO|Global tuberculosis report 2016. WHO (2017).
13. Notomi, T. et al. Loop-mediated isothermal amplification of DNA. 28, (2000).
14. Priye, A. et al. A smartphone-based diagnostic platform for rapid detection of Zika, chikungunya, and dengue viruses. Sci. Rep. 7, 44778 (2017).
15. Curtis, K., Rudolph, D., Nejad, I. & Singleton, J. Isothermal amplification using a chemical heating device for point-of-care detection of HIV-1. PLoS One (2012).
16. Damhorst, G. L. et al. Smartphone-Imaged HIV-1 Reverse-Transcription Loop-Mediated Isothermal Amplification (RT-LAMP) on a Chip from Whole Blood. Eng. (Beijing, China) 1, 324-335 (2015).
17. Song, J. et al. Instrument-Free Point-of-Care Molecular Detection of Zika Virus. Anal. Chem. 88, 7289-7294 (2016).
18. Toumazou, C. et al. Simultaneous DNA amplification and detection using a pH-sensing semiconductor system. Nat. Methods 10, 641-6 (2013).
19. Duarte-Guevara, C. et al. On-chip electrical detection of parallel loop-mediated isothermal amplification with DG-BioFETs for the detection of foodborne bacterial pathogens. RSC Adv. 6, 103872-103887 (2016).
20. Barker, R. H. et al. A simple method to detect Plasmodium falciparum directly from blood samples using the polymerase chain reaction. Am. J Trop. Med. Hyg. 46, 416-426 (1992).
21. Brambilla, D. et al. Multicenter Evaluation of Use of Dried Blood and Plasma Spot Specimens in Quantitative Assays for Human Immunodeficiency Virus RNA: Measurement, Precision, and RNA Stability Multicenter Evaluation of Use of Dried Blood and Plasma Spot Specimens in Quan. J Clin. Microbiol. 41, 1888-1898 (2003).
22. Arrayit Microarray Printing Pins and Printheads—Professional 946 Stealth ChipMaker™ Spotting Gene Chips Bead Array Manufacturing. Available at: http://www.arrayt.com/Products/Microarray_Printing/microarray_printing.html. (Accessed: 30 Apr. 2017)
23. A Flash Storage Technical and Economic Primer.flashstorage.com (2015).
24. Taiwan engineers defeat limits of flash memory. phys.org
25. Micron Technology, Inc.-3D XPoint™ Technology. Available at: https://www.micron.com/about/ourinnovation/3d-xpoint-technology. (Accessed: 30 Apr. 2017)

### Example 2: Detection of Bacteria in a Dried Blood Sample

Detecting extremely low target analyte or pathogen count (1-10 CFU/ml or lower) has been a fundamental challenge in the medical diagnostics¹ and food testing industry² due to extremely low signal to noise ratio. Cell culture, which usually take 1-5 days, is the only way of detecting these low pathogen levels in any sample.^{1,3,4} Sepsis, a clinical syndrome that results from dysregulated inflammatory response to infection leading to organ dysfunction has very high morbidity and mortality.⁴ It is also estimated to be the most costly inpatient diagnosis, accounting for more than $23 billion annually in US alone.⁵ Since sepsis usually has very low pathogen counts (1-3 CFU/ml)⁶ the only way for diagnosis is culture followed by nucleic acid amplification or direct molecular detection after a positive blood culture.³ Blood cultures (or other sample cultures) are very time consuming (1-5 days), have a very high false negative rate and do not work for fastidious pathogens such as Chlamydia pneumoniae which are harder to culture.^{3,7}

Our approach involves directly amplifying (nucleic acid amplification) target analyte from dried crude biological samples. We perform this amplification in a pixelated petri dish with an array of wells making the whole approach “Digital”—The number of wells which give positive amplification give the exact count of the copy number of analytes. This is a practical way of doing digital amplification with extremely high sensitivity (1 cfu/ml or lower) from crude, unprocessed samples, while processing large quantities (1-10 mL) of the biological sample. The processing of large amount of the biofluid is required to effectively sample the target analyte or pathogen which is present in very low concentrations. We have shown that for reactions from crude samples, bi-phasic reactions, consisting of separate dried sample phase and separate solution phase (with the amplification enzymes and reaction mix) have lower threshold times and are fundamentally superior compared to single phase (mixed) reactions where the crude sample is mixed with the amplification enzymes and buffers. This is likely because the interaction between inhibitory contaminants in crude samples and the amplification enzymes are reduced in the scenario where the sample is dried and does not mix with the enzyme solution above. We have also shown that this nucleic acid amplification reaction, post thermal lysis (universally applicable) of the target, initiates both in the solid phase (dried sample) and in the solution phase through the simultaneous diffusion of enzymes and buffer components into the dried sample cake and the diffusion of the target DNA (e.g. Pathogen DNA) into the solution above.

We start by drying the body fluid sample (blood, urine, saliva, stool, nasopharyngeal sample etc.), or food sample slurry (after grinding), in a pixelated petri dish with an array of wells. (The well numbers can range from 1e3 to 1e6 or higher, and, the well volume can vary from 10 picoliters to few nanoliters). The pixelation or partitioning of the sample into many (1e3 to 1e6 or higher) sub samples in separate wells helps in improving the signal-to-noise ratio and the efficiency of the reaction by performing independent reactions in each well. For example, if 1 ml of blood containing only 1 bacteria (starting concentration=1 bacteria/ml) is equally partitioned into 1e6 number of wells, then that one bacteria will end up going in one of the wells and the final bacteria concentration for that well will become 6 orders of magnitude higher (1 bacteria/nanoliter). The drying of the sample has the following roles:

1. Reduces the effective sample size to handle. For example, whole blood has approximately 50% plasma and so after drying, 1 mL of whole blood will become approximately 0.5 mL of dried blood cake.

2. Traps the target of interest in the wells.

3. Preserves the RNA/DNA for months at room temperature avoiding the need for cold chain for sample storage and stability. We confirm the stability of dried blood samples and repeatability of amplification from dried blood with spiked pathogens (E. coli or S. Aureus) for up to 28 days.

4. Drying of crude biological sample allows nucleic acid amplification to occur in the wells even with very high abundance of contaminants (For e.g.—Erythrocytes and other cell types in blood, Extracellular matrices etc.). The contaminants remain as a cake at the bottom of the wells behaving similar to a precipitate in the solution and have minimal effect on the reaction.

5. The dried sample tethers to the substrate (wells) and behaves like a single solid object which is easier to handle and store.

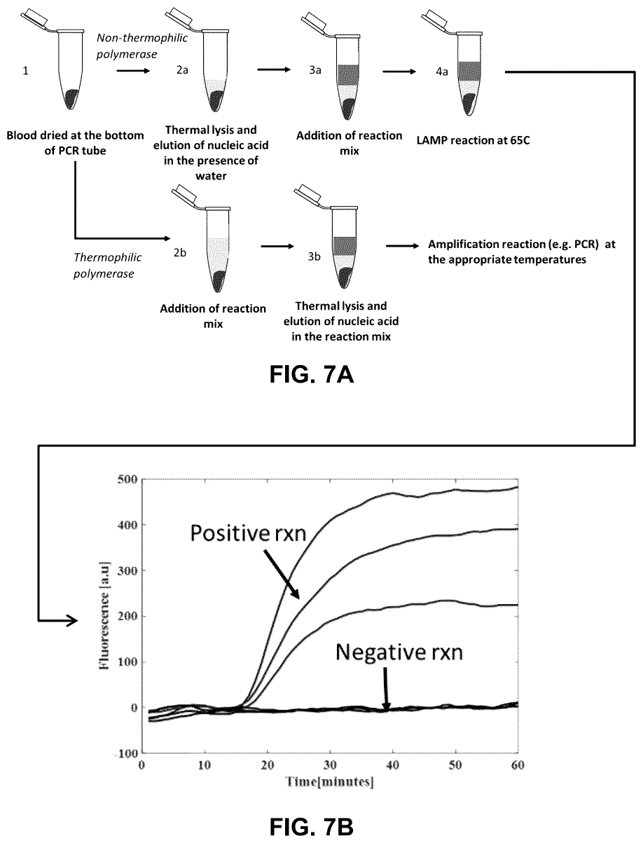

Bi-phasic amplification reaction: To validate the reaction mechanism of our bi-phasic reaction approach, we carried out tube-based loop mediated isothermal amplification reaction (LAMP) for the detection of wzy gene for the O157:H7 strain of E. coli in whole blood (FIGS. 7A-7B). Unprocessed whole blood spiked with the spiked E. coli was dried at the bottom of PCR tubes, followed by elution of bacterial DNA in the presence of nuclease-free water by thermal lysis at 95° C. Thermal lysis is a standard procedure of lysing cells (also works with viruses, and other pathogens) and eluting their nucleic acids (gram-positive and gram-negative bacteria).⁸⁻¹⁰ Upon lysis of the cells, the reaction mix is added and LAMP amplification is carried out at 65° C. In our bi-phasic reaction, the dried blood and associated contaminants at the bottom of the tube remains as a solid single phase with minimal diffusion into the reaction mix above. Inhibitors remain as precipitates at the bottom of the tubes, and do not become part of the reaction. The amplification reaction initiates both in the solid phase (dried sample) and in the solution phase through the simultaneous diffusion of enzymes and buffer components into the dried sample cake and the diffusion of the target DNA (e.g. Pathogen DNA) into the solution described herein.

Our bi-phasic reaction approach can be made compatible with any sample type (urine, saliva, food) to test for the presence of any nucleic acid markers via any nucleic acid amplification test (NAAT).

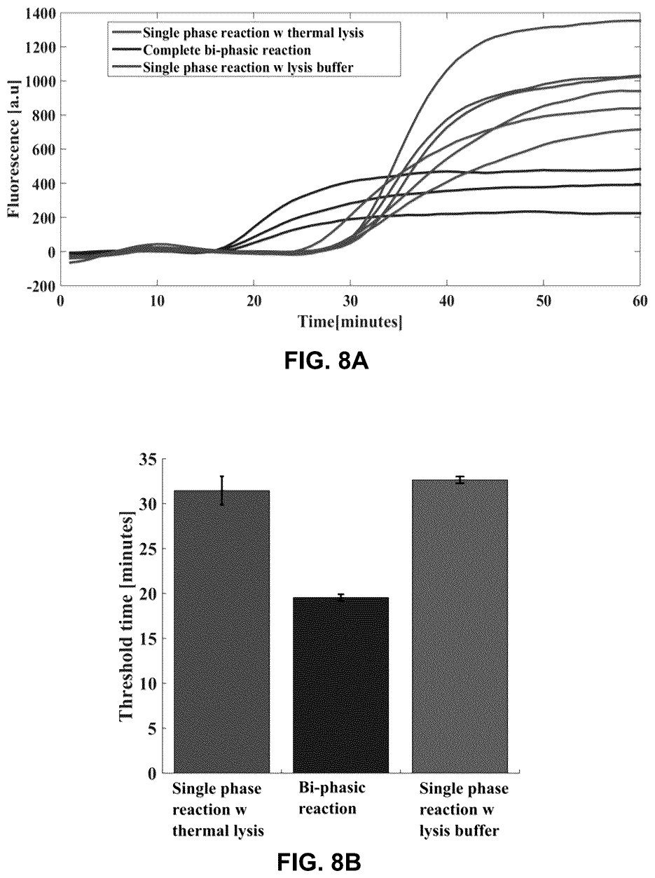

To validate that the described bi-phasic reaction is superior to other single phase reactions, we compare the reaction approach to other LAMP reaction techniques that use unprocessed whole blood. The final reaction concentration of the spiked pathogen was kept the same at 1.25e3 CFU/uL across all the splits tested. The bi-phasic reaction using the dried sample yielded much shorter threshold times compared to the two other alternate techniques. (FIGS. 8A-8B).

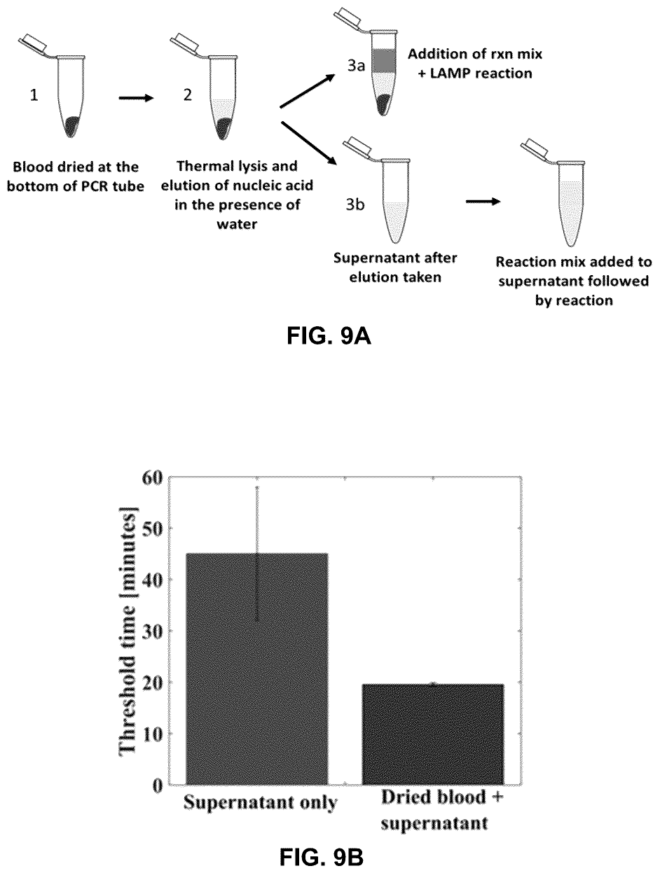

The bi-phasic reaction using dried blood is initiated in both the solid dried blood phase and the liquid phase (with the eluted DNA). (FIGS. 9A-9B) Reaction carried out with only supernatant water post elution gave much higher threshold times compared to the usual dried blood reaction (dried blood+supernatant solution). This indicates that not all of the pathogen DNA is eluted into the solution above the dried blood cake and indicates the presence of target DNA in the dried blood cake. The accessibility of the Polymerase enzyme to this trapped DNA in the dried blood cake is not an issue as evident by the consistent and repeatable threshold times across experiments. The final concentration of the O157.H7 strain of E. coli was 1.25e3 CFU/mL for both the experiments.

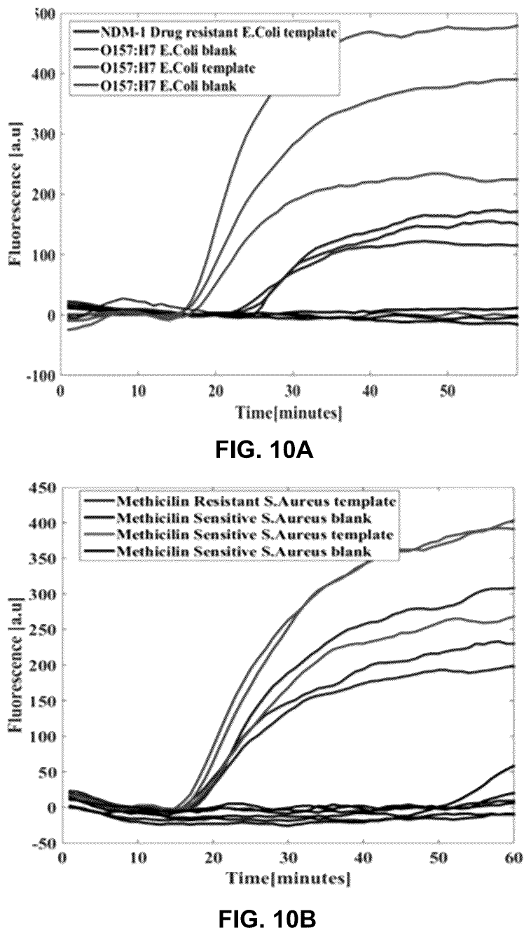

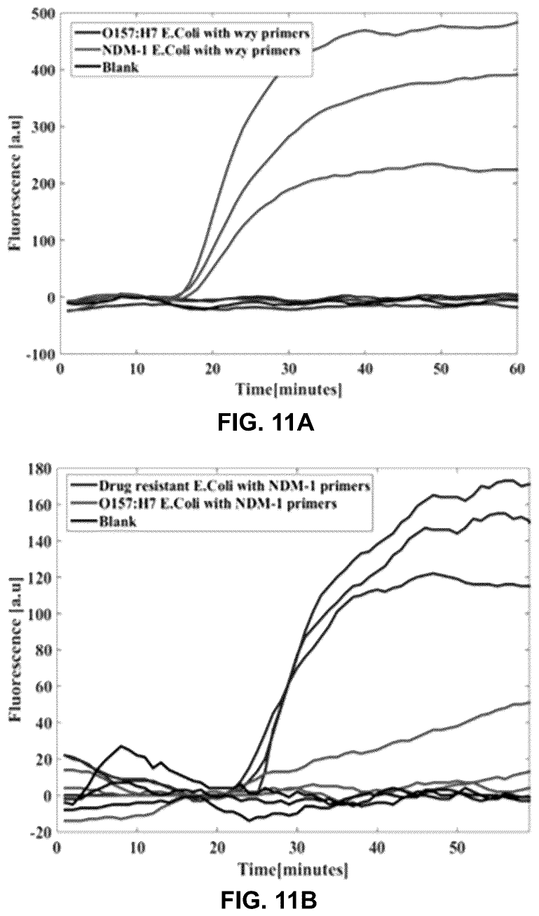

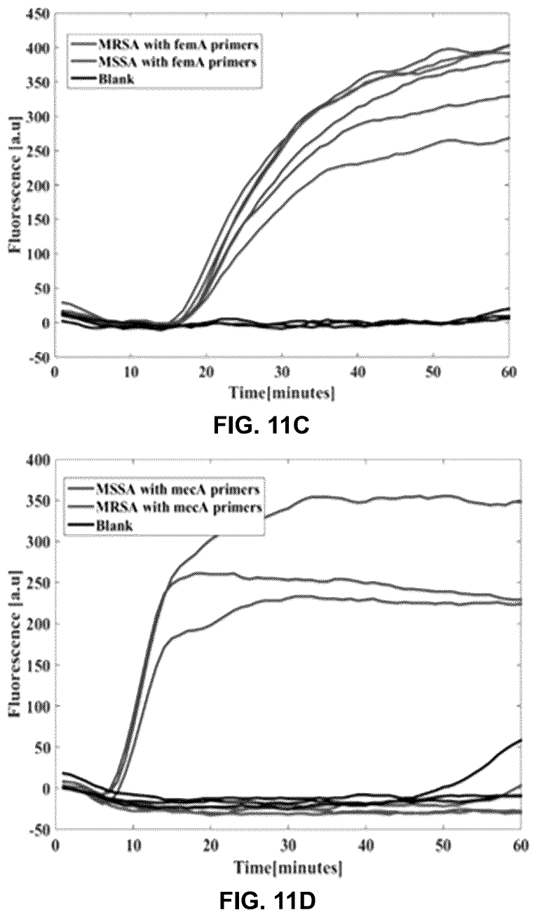

Bi-phasic reaction with different pathogens: Elution protocol via the thermal lysis proved to be efficient for the detection of both gram-negative and gram-positive using our bi-phasic reaction approach. LAMP reactions were carried for the detection of two gram-negative bacteria (carbapenem sensitive and carbapenem-resistant of E. coli), and two gram-positive bacteria (methicillin sensitive and methicillin resistant strain of S. Aureus). (FIGS. 10A-10B) The detection of these targets was specific and yielded no non-specific amplification. The cross-reactivity of each primer sets was also confirmed using our bi-phasic reaction approach (FIGS. 11A-11D).

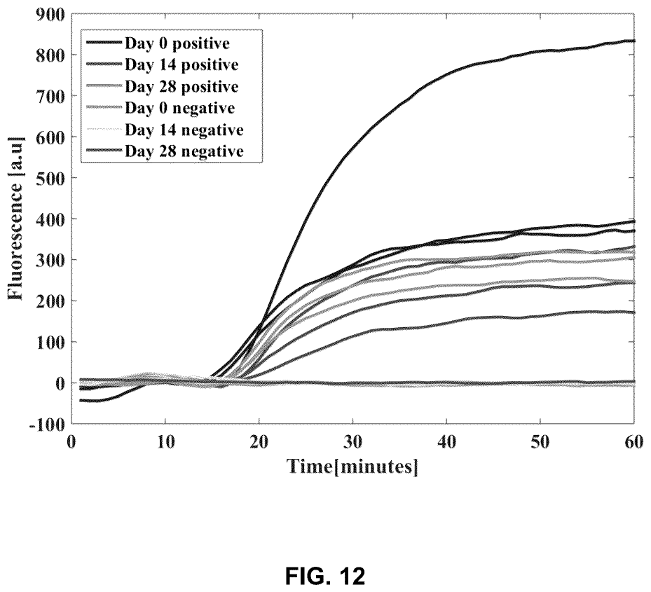

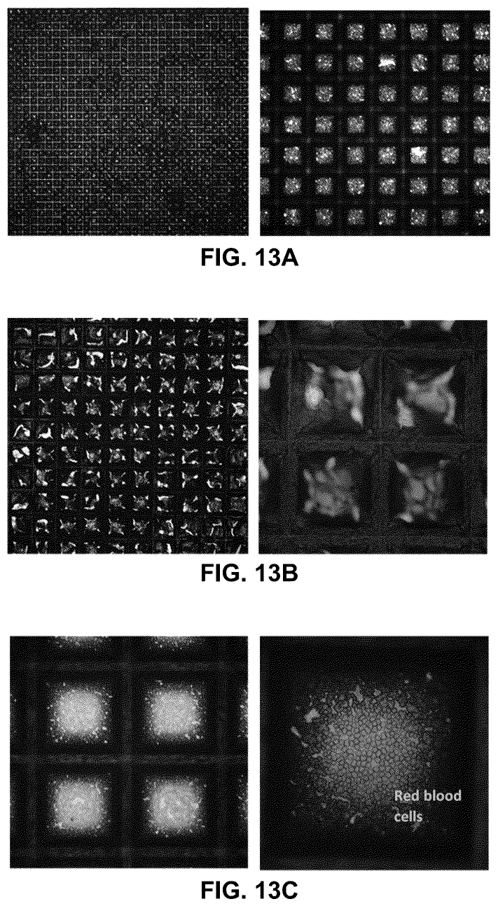

On-chip digital amplification reaction: To increase the sensitivity of the bi-phasic reaction, the bi-phasic reactions are combined with digital nucleic acid amplification reaction. The digital amplification is carried out by reaction in microfluidic chips (silicon, glass, plastic) with >1e6 wells. The sample is loaded into the million wells and then dried on our chip. The low starting pathogen count, low volume of liquid per well and large number of partitions implies that there is either 1 or 0 target (pathogen) per well (poison statistics for digital amplification). Moreover, the low volume per well increases the effective concentration of the template in the positive reaction. The digital amplification reaction can be monitored end-point using a fluorescent microscope or a portable fluorescence reader. The number of positive wells, characterized by greater fluorescence at the end-point, indicates the number of pathogens/target in the starting sample. The blood loading protocol is optimized on-chip to achieve uniform filling per well with minimal cross-talk between wells. (FIG. 12.) The chips are made hydrophilic (using Amino silane or PLL), and an excess amount of whole blood is loaded onto the chip to ensure all the wells were filled with blood. Upon complete filling, excess blood is removed by blowing air with a N₂ gun. The blood equivalent to a well volume is retained in each well due to dominant capillary forces and the excess is removed due to air pressure. This rapid sample loading technique takes only a few seconds to load greater than million wells (with picoliter to nanoliters of volume) homogenously. Then, the blood is allowed to dry, and chip filling was qualitatively assessed via optical microscopy.

Enzyme for amplification reaction: After loading the blood on chip, the target DNA/RNA is eluted in the reaction mix at 95° C. for 1-2 minutes. The reagent loading may be done in either a one-step, or, a two-step process depending on the thermal stability of the polymerase. In the case of polymerase chain reaction which requires thermal cycling, or isothermal amplification reactions utilizing a thermophilic enzyme (stable at 95° C. for a few minutes), the reaction mix with the enzymes is added to the wells with dried blood and the nucleic acids are eluted at 95° C. For amplification reactions not using a thermostable polymerase, such as LAMP Bst polymerase which gets denatured above 70° C., the polymerase is introduced into reaction mix separately after the DNA/RNA elution step via thermal lysis. The elution step in such scenario is done in an amplification compatible buffer mix containing all the ingredients for amplification except the enzyme (everything except enzymes are thermostable at 95° C.).

There are two broad strategies for the introduction of non-thermophilic polymerase post DNA/RNA elution as follows:
a. Bead-based lyophilized enzyme delivery
b. Lyophilizing enzymes on micropillars and introducing into the wells as a cover to the chip

Both the techniques utilize the lyophilization of polymerase enzymes either on beads or micropillars.

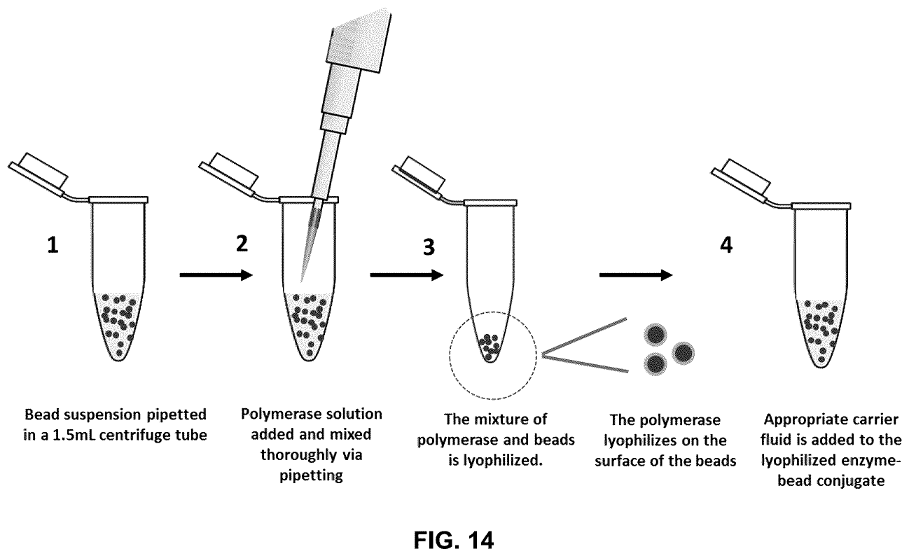

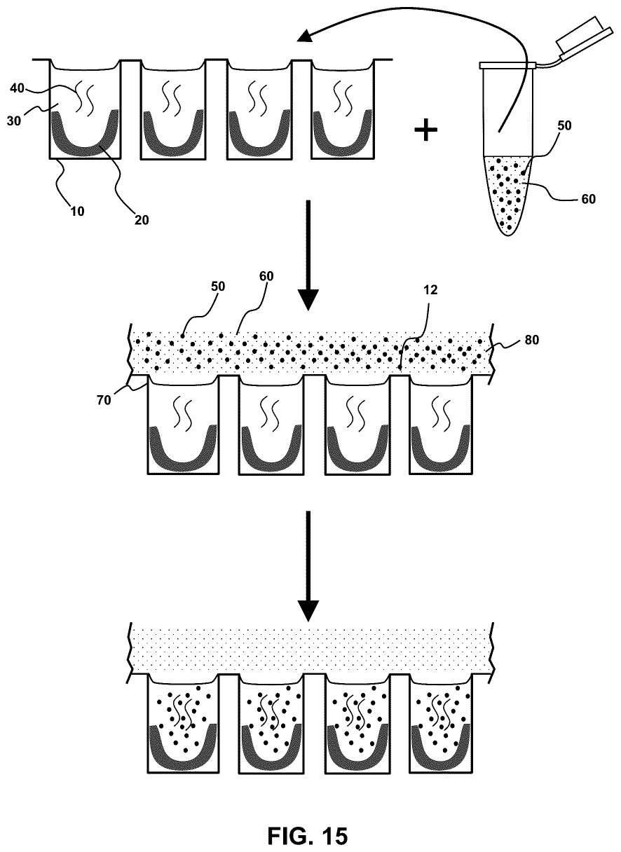

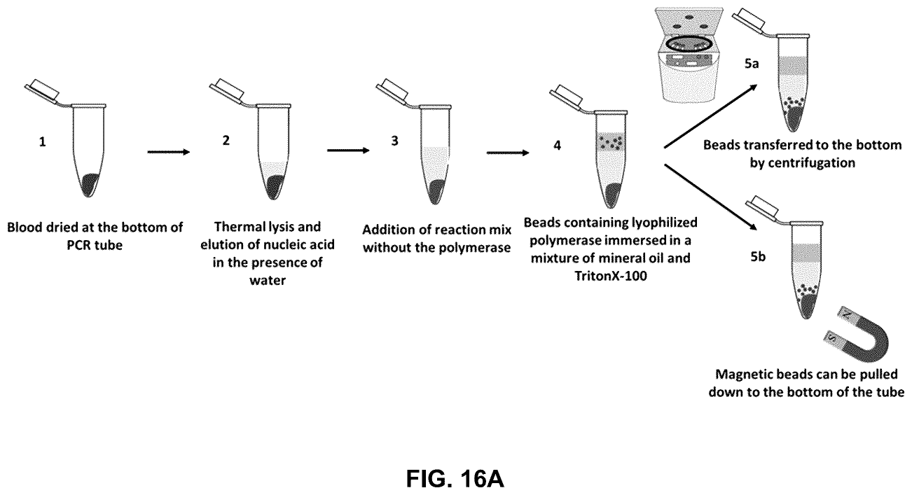

Bead-based enzyme delivery: FIG. 14 show the principle for lyophilizing enzymes on beads. The large number of beads per tube minimizes the lyophilization of beads on tube walls and maximizes enzyme retention on beads. Once the enzymes have been lyophilized on beads, a carrier fluid is used to pick up the beads and transfer them on chip and into the wells. The considerations for this step are:

1. A solvent that is miscible or soluble with the reaction mix (already in wells) may not be directly used to pick up the beads. Since the wells are already filled with buffer mix in the prior nucleic acid elution step, adding more of soluble/miscible solvent will cause overflow of the solvent and allow cross-talk between adjacent wells. This violates the independent well criteria required for independent and isolated reactions.

2. An immiscible solvent may be used which is lower in density than reaction mix solution to pick up the beads. This allows the carrier fluid to float on top of the wells while the denser beads with enzymes can fall into the underlying wells.

3. A miscible solvent (soluble with water/reaction mix) may be used as a carrier fluid if we have a separating layer of immiscible fluid in between. See the tri-layered approach described herein for explanation.

4. A miscible solvent can be used if the reaction mix is frozen post elution and the bead loading is done in sub-zero temperatures (degree C.). This prevents the molecular diffusion and mixing of the 2 miscible liquids (reaction mix and carrier fluid). The freezing temperature of the carrier fluid in such scenario should be below 0° C. and below the operating temperature of the process, so it remains as a liquid even when the reaction mix is frozen.

Based on the above criteria, the bead based polymerase delivery can be divided into 3 sub-categories.
i. Bi-layered approach (uses immiscible (with reaction mix) solvent as carrier fluid).
ii. Tri-layer approach (uses miscible/soluble solvent as carrier fluid with a separating immiscible fluid in between).
iii. Loading at sub-zero temperatures (uses low freezing point miscible/soluble solvent as carrier fluid at sub-zero temperatures).

Bead-based bi-layered approach: In the bi-layered approach to introduce polymerase into the reaction wells, the lyophilized beads are suspended in a mixture of a carrier fluid and a surfactant. The carrier fluid is both immiscible in and lighter than water so that it can stay afloat on top of the buffer mix in our microfluidic chip. The surfactant allows good mixing of the beads in carrier fluid by reducing the surface energy of the beads and keeping them de-agglomerated in the carrier fluid (otherwise the interactions between polar lyophilized beads and non-polar carrier solvent are not energetically favorable resulting in poor mixing). The lyophilized beads are polar (due to the enzymes present on them), and they are solubilized in a non-polar solvent (immiscible liquid—e.g. mineral oil, hexane, toluene) through the formation of nanoscale reverse micellar structures by the surfactant molecules. The mechanism for the polymerase lyophilization is described in FIG. 14. Both surfactants were tested with both low hydrophile-lipophile balance (HLB) such as PEF-2000, and high HLB such as Triton-X 100.

The on-chip schematic with the process flow (steps 1-4) for this bi-layer approach is illustrated in FIG. 15, illustrating the system having a plate with a plurality of microwells 10, dried fluid sample 20, liquid phase 30 in the microwells 10 and target nucleic acid 40. Beads 50 are provided in barrier layer (also referred to as a carrier fluid) 60. The beads are forced into the liquid phase, such as by application of release force, e.g., centrifugation or passive settling under gravity. In the top panel, wells with dried blood are filled with buffer mixture, after DNA/RNA elution, as illustrated by nucleic acid 40 in the buffer mixture liquid phase 30. A reagent (also referred herein as a biomolecule), such as a lyophilized enzyme on beads 50 suspended in an immiscible carrier fluid 60, is introduced to the plate with wells 10. The middle panel illustrates carrier fluid 60 with beads 50 to form a barrier layer (also referred herein as a hydrophobic substrate 80) over a top portion 70 of the microwell 10. Adjacent microwells 10 are separated by a separation surface 12. The bottom panels shows beads pulled from the hydrophobic substrate to the wells using centrifugation or other force, such as magnetic force for magnetic or magnetizable beads 50. In this manner, the hydrophobic substrate 80, with beads that may have a biomolecule, connects to the microwell separation surface 12 to fluidically seal adjacent microwells and prevent fluid transmission between microwells, thereby reliably preventing unwanted cross-talk.

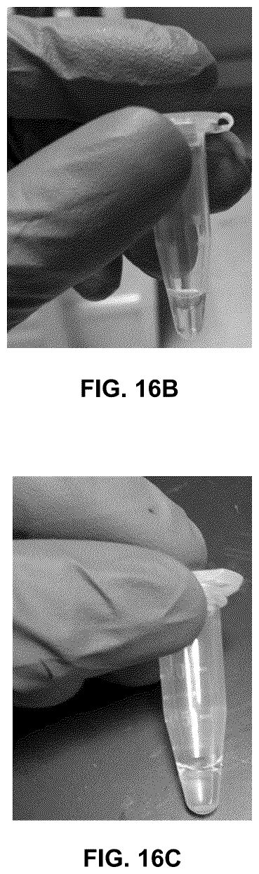

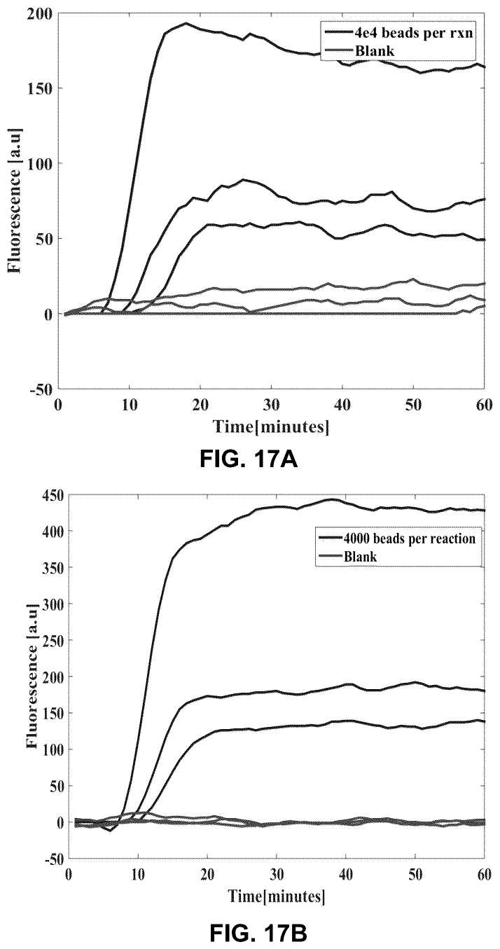

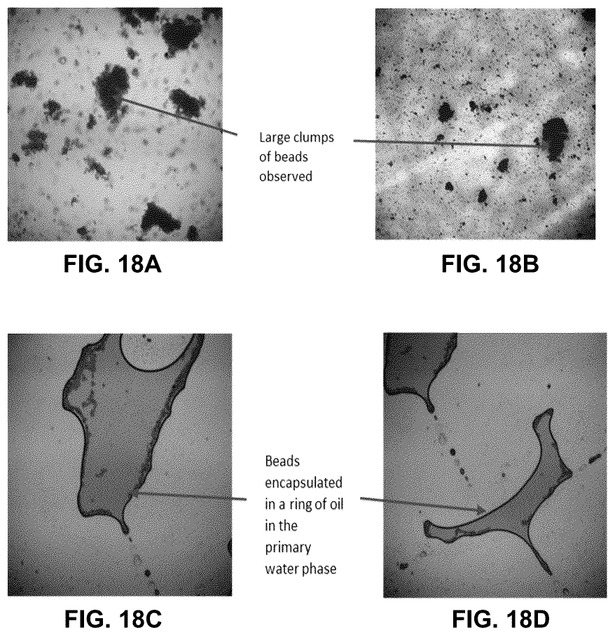

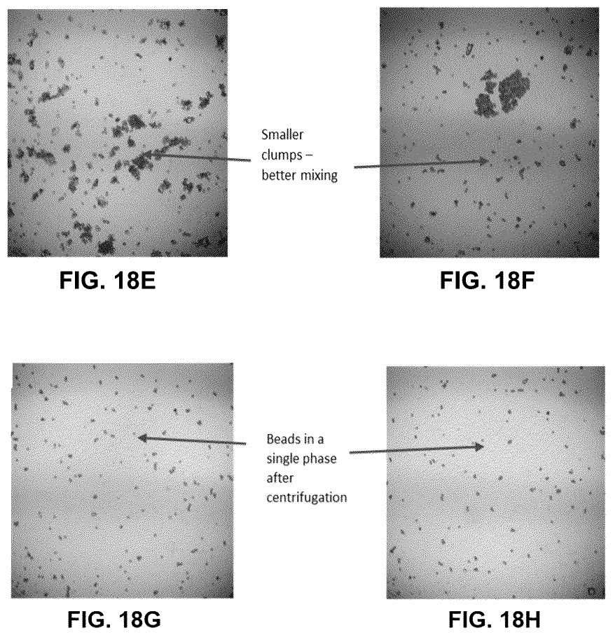

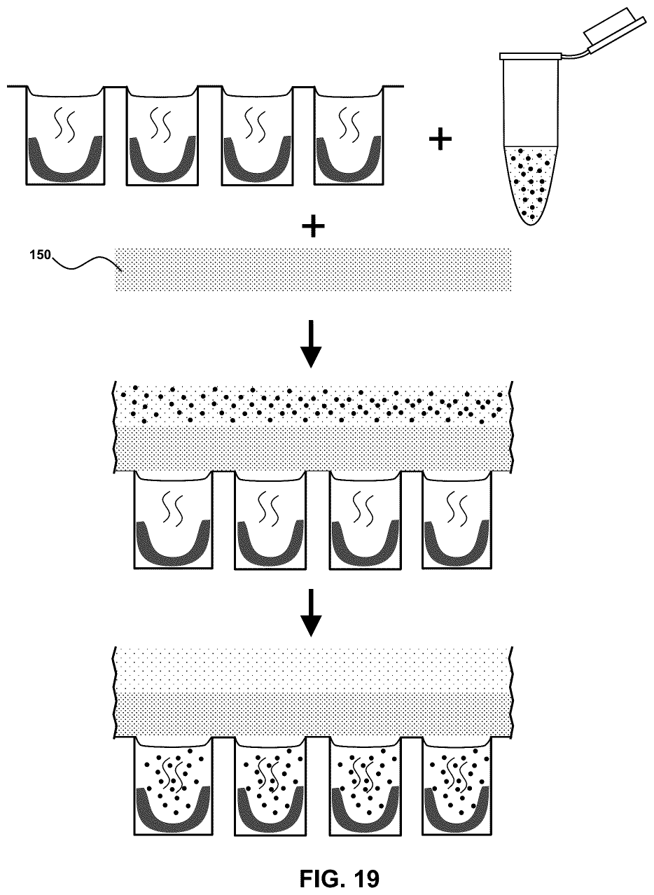

To validate this technique, a tube-based LAMP amplification reaction was carried out using this setup (FIGS. 16A-16C) using a mixture of mineral oil (other hydrocarbon based solvents such as hexane or toluene are also compatible) and Triton-X 100 as the carrier fluid. The beads are pulled to the bottom of the reaction either via centrifugation for polystyrene beads or via a strong magnet when using magnetic beads. LAMP amplification curves validating this technique and the microscopic validation of this technique are shown in FIGS. 17A-17B and FIGS. 18A-18H respectively.

Bead-based tri-layered approach: The tri-layered approach utilizes a polar solvent, such as ethanol which can solubilize beads easily but is less dense than water and the immiscible middle layer (e.g. mineral oil) as the carrier fluid. The bottom-most layer of the tri-layer system is water. The middle layer is an immiscible liquid lighter than water with or without a surfactant (similar to the top layer in the bi-layer approach). The topmost layer is the lyophilized beads suspended in a mixture of surfactant and a polar solvent. The topmost layer must be immiscible and less dense than the middle layer. It is important to note that the stability of polymerase enzymes may be compromised in solvents such as ethanol.¹¹ When using solvents such as ethanol which have the required low density for this method, the enzymes need to be protected from such solvents due to the solvents' denaturing effects on proteins. The protection of enzymes in denaturing solvents such as ethanol is shown by forming nanoscale micellar structures surrounding the bead with lyophilized enzymes through the use of surfactants. In this method, the surfactant in water (0.1% Triton-X 100-water-based) was first added to the lyophilized beads to initially form micelles around the beads with lyophilized enzymes. The ethanol was added after this step. This sequence of steps showed positive amplification. On the contrary, when ethanol came into direct contact with the enzymes, without any prior presence of the surfactant, no positive reaction was observed. The protection of enzymes in the presence of denaturing solvents was tested to be effective for 4 hours (not tested for more than 4 hours).

The on-chip schematic with the process flow (steps 1-4) for this bi-layer approach is illustrated in FIG. 19, where wells with dried blood, filled with buffer mixture and eluted nucleic acid are combined with lyophilized enzyme on beads suspended in a carrier fluid, such as ethanol, and a surfactant. Immiscible separating layer 150 forms an additional barrier layer, with the carrier fluid positioned on top of layer 150. Beads with desired reagents are pulled into the wells. This is referred to as a tri-layer, with two barrier layers and a liquid in the wells together forming three separate and well-defined liquids.

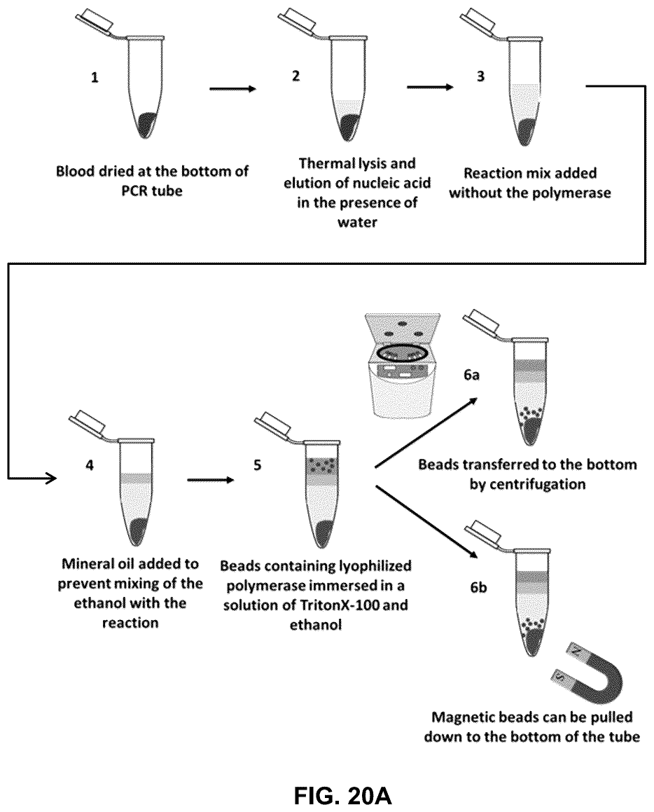

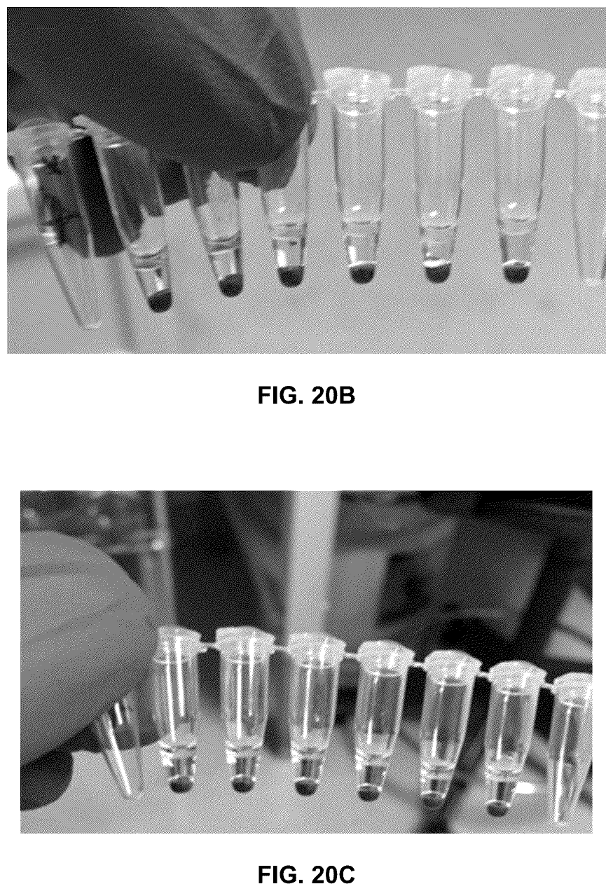

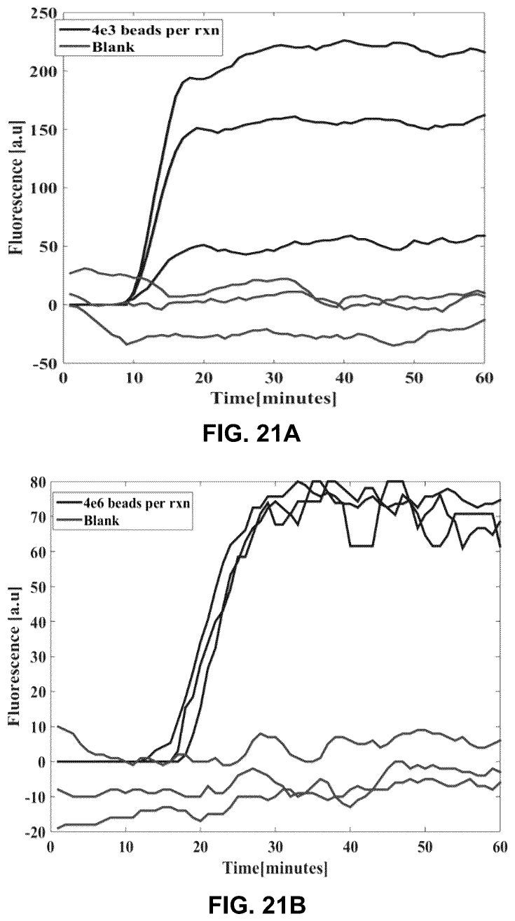

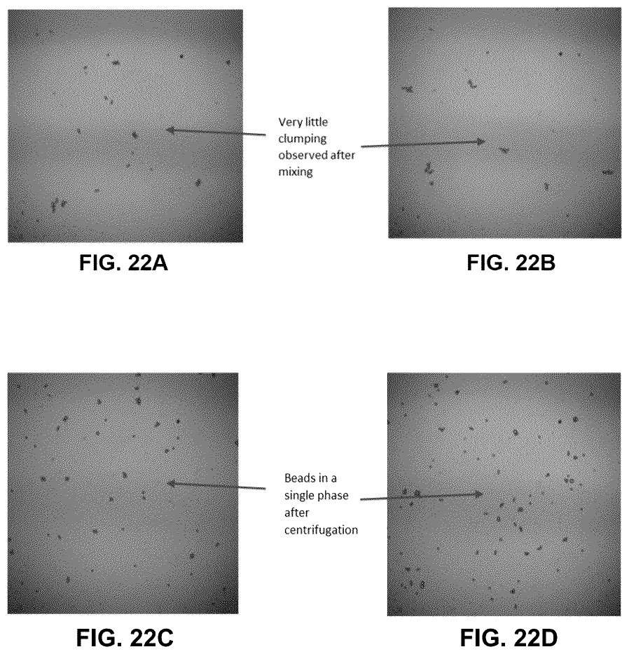

To validate this technique, we have carried out LAMP reactions where 1% Triton-X 100 followed by the addition 100% ethanol served as the carrier fluid for the beads. The beads were pulled down into the reaction mix in the same ways described in the bi-layered approach herein. The reaction schematics, LAMP amplification curves, and optical microscopy validation of the technique are shown in FIGS. 20A-20C, FIGS. 21A-21B and FIGS. 22A-22D, respectively.

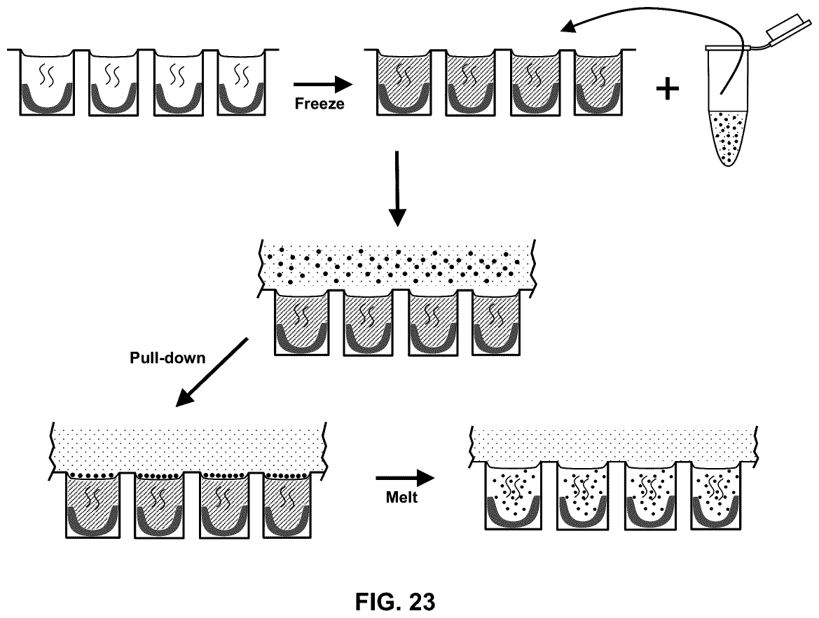

Loading at sub-zero temperatures: A miscible solvent can be used if the reaction mix is frozen post elution and the bead loading is done in sub-zero temperatures (degree C.). This prevents the molecular diffusion and mixing of the 2 miscible liquids (reaction mix and carrier fluid). The freezing temperature of the carrier fluid in such scenario should be below 0° C. and below the operating temperature of the process, so it remains as a liquid even when the reaction mix is frozen. Upon freezing of the reaction mix in wells at the appropriate temperature, a cold centrifugation or a magnetic pull-down (when using magnetic beads) will be carried out to pull down the beads to the interface of the frozen layer. The top layer, which will remain a liquid at the operating temperature, can then be removed by pipetting, and/or doing a series of cold washes, and/or letting it evaporate at sub-zero temperatures. After removal of the top layer, the bottom layer is thawed and the beads will be automatically incorporated into the reaction. The schematics for the on-chip process flow (steps 1-6) for this approach is illustrated in FIG. 23. The top panel illustrates freezing of the wells with buffer mixture and dried sample. Addition of the carrier fluid with beads results in a barrier layer on the top surface and beads pulled onto the top available frozen surface. Upon melting, the beads are pulled into the wells. The carrier fluid freezing point is lower than that of the water-based buffer or reaction mixture, to ensure the barrier layer is not frozen even if the liquid in the wells are frozen.

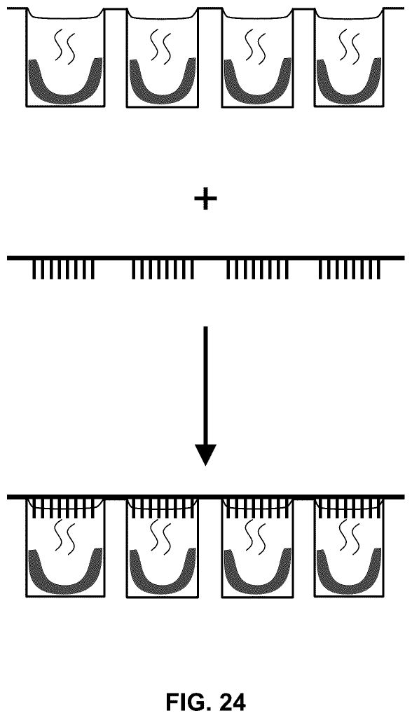

Micropillars based amplification enzyme delivery: In this approach, needle-like micropillar structures are fabricated where polymerase enzymes will be lyophilized, more generally referred to as microstructures. Once the target nucleic acid has been eluted in the buffer mix on-chip in filled wells, the microneedles with the lyophilized polymerase are aligned and interfaced with the chip from the top. Each well can interact with multiple micro-needles. The lyophilized polymerase will hydrate and diffuse into the reaction once the needles come in contact with the buffer mix. The micro-needles can either be removed or clamped to the microfluidic chip to make one final device. The schematics for the process flow micropillars-based enzyme delivery are illustrated in FIG. 24. Briefly, wells with dried fluid sample, such as blood, are filled with a liquid phase, such as a buffer mixture. Nucleotide target (e.g., DNA/RNA) has been eluted from the sample (top panel). Micropillars with lyophilized enzymes (middle panel) are used to cover the wells (bottom panel). The micropillars introduce the enzyme into the reaction mixture while also forming a seal to prevent evaporation and unwanted cross-talk between wells.

Bead loading through emulsion: In yet another version, an emulsion of polar solvent in another immiscible solvent is prepared where the beads will be in the compatible polar phase. Then this emulsion be used a carrier fluid for bead loading.

### REFERENCES

1 A. Perner, A. Gordon, D. De Backer and G. Dimopoulos, Intensive care, 2016.
2 F. Wang, Q. Yang, J. A. Kase, J. Meng, L. M. Clotilde, A. Lin and B. Ge.
3 R. P. Peters, M. A. van Agtmael, S. A. Danner, P. H. Savelkoul and C. M. Vandenbroucke-Grauls, Lancet Infect. Dis., 2004, 4, 751-760.
4 J. Vincent, S. Opal and J. Marshall, Lancet (London, 2013.
5 A. B. Martin, M. Hartman, J. Benson and A. Catlin, Health Aff, 2016, 35, 150-160.
6 A. J. M. Loonen, M. P. Bos, B. van Meerbergen, S. Neerken, A. Catsburg, I. Dobbelaer, R. Penterman, G. Maertens, P. van de Wiel, P. Savelkoul and A. J. C. van den Brule, PLoS One, 2013, 8, 1-7.
7 N. Mancini, S. Carletti, N. Ghidoli and P. Cichero, Clin. Microbiol., 2010.
8 M. S. Islam, A. Aryasomayajula and P. R. Selvaganapathy, 2017.
9 M. M. Packard, E. K. Wheeler, E. C. Alocilja and M. Shusteff, 2013, 105-116.
10. O. M. De Bruin and H. C. Bimboim, BMC Microbiol., 2016, 1-10.
11 R. Chaloupkova and J. Damborsky, 2013.
12 A. Ganguli, A. Omob, H. Yu, G. L. Damhorst, W. Chen and F. Sun, 2017, 1-13.

### Example 3: Lyophilized/Dried Biomolecule Delivery to Micro/Nano-Well Arrays and Strategies for End-Point Multiplexed Detection of Nucleic Acid Targets

To deliver material, including biomolecules, enzymes and the like, into a micro-well array in a multi-step process, the material can be lyophilized or dried on a fluid barrier, such as beads-on-a-wax paper, and pulled down into the wells using centrifugation or magnetic pull down. Since in a micro-well array, cross contamination between two adjacent wells is a challenge, loading the enzyme through beads on a wax paper allows simultaneous loading of a large array of wells (>1e6) in a single step requiring less than 1 minute. The fluid barrier may be a substrate with a hydrophobic coating, such as wax paper with its hydrophobic wax coating. Various barriers are compatible with the methods and systems provided herein, so long as the barrier prevents any fluid exchange between adjacent wells during the process.

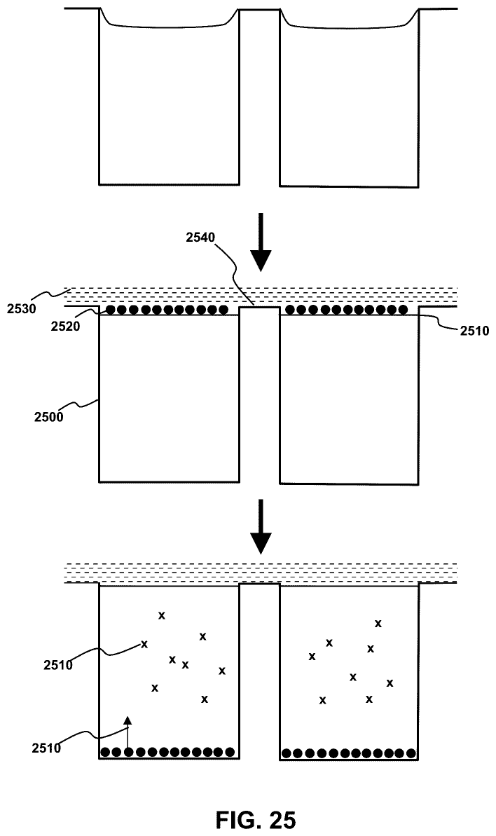

A schematic illustration of the beads-on-barrier example is provided in FIG. 25. In this example, lyopholized/dried biomolecules are provided on beads 2520, with the beads connected to a hydrophobic substrate, so as to provide a platform for homogeneous delivery of material into a microwell array, including reliable delivery to individual microwells of the array. Reagents 2510 are provided in the microwells 2500. Material 2520, illustrated as beads containing lyopholized/dried biomaterial, is connected to a hydrophobic substrate 2530 provided to the top 2501 of the microwells. The substrate 2530 functions to fluidically seal 2540 adjacent microwells, thereby preventing any unwanted cross-talk between adjacent microwells. Application of a release force 2550 releases the biomolecules (reagents) 2510 to the microwell. The biomolecules may be actively dispersed in the well by a mixing force and/or may passively disperse by diffusion.

Advantages of a process such as the one illustrated in FIG. 25 include: (1) Loading lyophilized enzymes/biomolecule while preventing any cross-talk between adjacent wells; (2) Sequential loading can allow multiple loading steps, each taking a relatively short time, such as less than a minute; (3) Uniform distribution of loading achieved as the distribution of beads on paper substrate is the same as the distribution transferred into the micro-well array. Of course, as desired, the beads may be distributed over the substrate release surface as desired, including in a non-uniform or spatially varying pattern, so that certain wells receive more biomolecule compared to other wells; and (4) No need for any other solvent/liquid for the loading process.

### Example 4: Multiplexing Using Enzymes as End Point Detection Step for a High Number of Targets, Including CRISPR Proteins

For digital amplification from dried whole blood in a micro-well array, we can use the CRISPR-cpfl (or similar protein/biological molecules) for an end-point detection step of a high number of targets. The digital amplification chip isolates different targets in different wells allowing unique detection reactions in each well. Unique Crispr-cpfl enzymes are designed for each nucleic acid target and then the target specific crispr-cpfl enzymes are lyophilized on different sets of beads. The enzymes on beads are dispersed in an immiscible solution using surfactants and sequentially introduced into the digital amplification chip. Other methods of enzyme introduction into the microwell array are also compatible. After each step of introduction of the target specific crispr-cpfl beads, the chip is measured for any positive detection. After the first round of bead introduction and detection, the next round begins with a different target-specific crispr-cpfl beads. This can be repeated as many times as desired, such as for up to 100 targets, or even more than 100 targets, and each detection takes a very short time since the on-chip reaction volumes are very low (nanoliters to picoliters) and it will be easy for the cpfl to quickly find its target in that volume (effective target concentration become high since volume becomes low after sample partitioning). A preamplification step to increase the copy numbers of the target can be performed to aid in the detection process. This technique is compatible with any sample digitization setup, with or without amplification. When combined with digital amplification it can be used to detect extremely low pathogen concentration (1 copy/ml or lower) for any number of applications including, for example, Sepsis diagnosis.

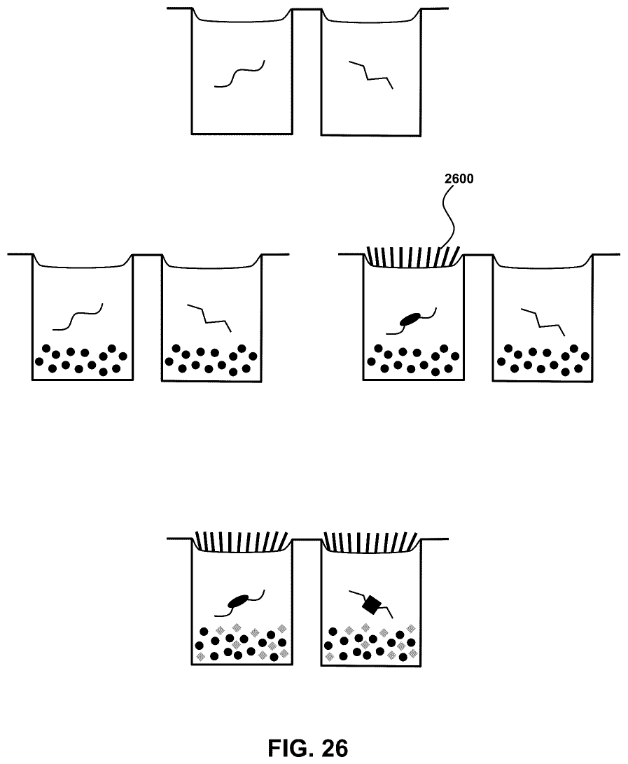

This example is schematically illustrated in FIG. 26, where a single target is provided in each well by sample digestion-sample partitioning. The remaining steps show sequential delivery of target specific Crispr-cpfl biomolecules in a microwell array, including by sequential delivery by the sequential process of FIG. 25. After each delivery of a Crispr-cpfl enzyme system, the array is scanned for fluorescence and a positive fluorescent signal from any well indicates the presence of a target corresponding to the delivered cpfl. This sequential delivery and detection can be done for a large number of targets, such as up to 100 to 1000, 100 to 200, or greater than 100. Large target numbers are achieved by selecting the bead volume as about 3-4 orders of magnitude lower than the well volume. FIG. 26 shows Crispr-Cpfl for multiplexed detection of nucleic acid targets in a digitized platform. Two wells are illustrated with different targets. Sample digitization-sample partitioning achieve a one target molecule per partition (e.g., well) (top panel). The remaining panels show sequential delivery of target specific crisp-cpfl biomolecule in a microwell array. After each delivery, the array is optically scanned for fluorescence, and a positive fluorescent signal 2600 from a well indicates the presence of a target corresponding to delivered cpfl. Sequential delivery and detection can be performed for a large number of targets (e.g., >150) by selecting a bead volume which is 3-4 orders of magnitude lower than the cell volume (bottom panel).

Advantages of this multiplexing example includes: (1) This technique allows combining digital amplification (PCR, LAMP, etc) in microwell array (or other formats) with end-point highly multiplexed and specific detection of the targets. In the digital format, the detection would also serve as quantitation since statistically only 1 or 0 copy of the target nucleic acid is present per well; (2) One cycle can be defined as loading of target-specific crispr-cpfl followed by fluorescence detection/scanning of the array. N such cycles can be repeated, where N is up to 100 or even greater than 100. The wells lighting up (fluorescing) for a given cycle number will have the corresponding target molecule. (3) Crispr-cpfl, crispr-cas13 and similar proteins bind specifically to their target molecule through their “engineered guide RNA” and once bound, they start degrading/cutting any and all unbound nucleic acid molecules. Hence, a reporter nucleic acid molecule with a quenched fluorescence (quencher) can be used, whose fluorescence is retrieved once the molecule is cut by the cpfl or similar enzyme. (4) A pre-amplification step will increase the copy numbers of the target per well and reduce the time to detection for each cycle/target. (5) No two targets can be in the same well as only 1 target would be detectable in such case and the other degraded by the active enzyme. Hence, there is a need for sample digitizing and partitioning system (droplet/well etc) to perform multiplexed end point detection. (6) Crispr-cpfl can be replaced by other target specific hybridization strategies such as fluorescence, in situ hybridization, and the like.

### Example 5: A Culture-Free Biphasic Approach for Sensitive and Rapid Detection of Pathogens in Dried Whole Blood Matrix

Antibiotic therapy within 1-3 h of initial symptom-based recognition can significantly reduce mortality in BSI and bacteraemia. However, blood cultures which remain the gold standard for pathogen detection, can take up to 5 days for a confirmed negative result. Here, we report a culture-free “biphasic” approach to performing amplification reactions directly from whole blood, including large volume whole blood, including with detection at less than 3 hours. “Large volume”, in this context, refers to a whole blood sample that is greater than 0.5 mL, greater than 10 mL, including between 0.5 mL and 50 mL, and any subranges thereof. We dry the blood and create a physical micro and nanoscale fluidic network inside the dried blood matrix to allow for DNA amplification. We show single molecule sensitivity (LOD=1.2 cfu/mL) for E.coli, MRSA, MSSA, and C. Albicans (0.8-1 mL of blood, sample-to-result time <2.5 h). We validate the assay with clinical samples and found complete agreement with the results of the clinical laboratory that used blood culture and PCR.

Blood stream infections (BSIs) cause high mortality and their rapid detection remains a significant diagnostic challenge. Timely and informed administration of antibiotics can significantly improve patient outcomes. However, blood culture, which takes up to 5 days for a negative result, followed by Polymerase Chain Reaction (PCR), remains the gold standard in diagnosing BSI. This example provides a new approach to blood-based diagnostics where large blood volumes can be rapidly dried resulting in inactivation of the inhibitory components in blood. Further thermal treatments then generate a physical micro and nanoscale fluidic network inside the dried matrix to allow access to target nucleic acid. The amplification enzymes and primers initiate the reaction within the dried blood matrix through these networks precluding any need for conventional nucleic acid purification. High heme background is confined to the solid phase, while amplicons are enriched in the clear supernatant (liquid phase) giving fluorescence change comparable to purified DNA reactions. We demonstrate single molecule sensitivity using loop-mediated-isothermal-amplification reaction (LAMP) in our platform by detecting broad spectrum of pathogens including gram-positive methicillin-resistant and methicillin-susceptible S.aureus bacteria, gram-negative E. coli bacteria, and Candida albicans (fungus) from whole blood with a LOD of 1.2 cfu/mL from 0.8-1 mL starting blood volume. We validate our assay using 63 clinical samples (100% sensitivity and specificity), and significantly reduce sample-to-result time from over 20 h to <2.5 h. The reduction in instrumentation complexity and costs compared to blood culture and alternate molecular diagnostic platforms has broad applications in healthcare systems, including in both developed world and in resource-limited settings.

Fast and accurate identification of infection causing microorganisms in blood remains a significant diagnostic challenge¹. Bloodstream infections (BSI) are often associated with severe diseases and result in high morbidity and mortality, especially in critically ill patients². Sepsis, a life-threatening organ dysfunction caused by a dysregulated host response to infection, is the most important diagnostic and therapeutic challenge from BSIs³. Sepsis is currently the most expensive condition treated in US hospitals with its nonspecific diagnoses alone accounting for US $23.7 billion every year^{4,5}. Despite that, there is an alarming 31% increase in sepsis related deaths between 1999 and 2014⁶. Moreover, neonates comprise an additional group vulnerable to BSIs due to their deficient adaptive immune responses. Twenty five percent of all neonates admitted to neonatal ICU are diagnosed with Sepsis and 18-35% of these end up dying from pathogen infection⁷⁻⁹.

Sepsis generally results from a primary bacterial infection, or less frequently from fungal and/or viral infection¹. It has been shown that timely administration of appropriate antibiotics significantly improves patient outcomes^{10,11}. However, the current clinical gold standard for diagnosing sepsis/BSIs remains blood culture followed by nucleic acid amplification and detection using Polymerase Chain Reaction (PCR). The blood culture step is too slow and cumbersome to allow for initial management of patients and thus contributes to high mortality¹²⁻¹⁴. Moreover, in the absence of timely results from robust diagnostic tests, the patients are administered highly potent broad-spectrum antibiotics without any patient stratification, increasing anti-microbial resistance, and emergence of drug-resistant and atypical pathogens^{15,16}.

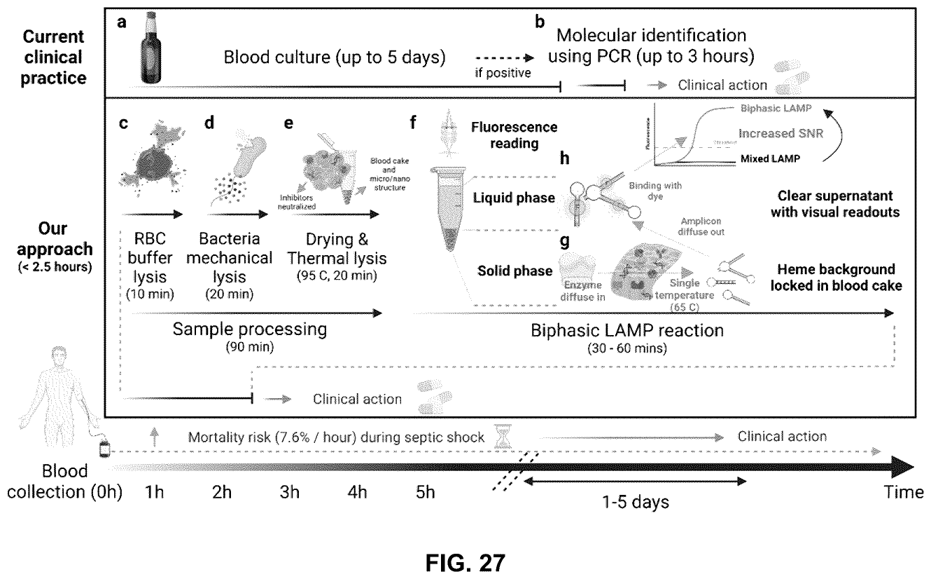

Blood culture for diagnosing blood stream infections requires the culture of viable pathogens for up to 5 days (FIG. 27—left, top panel)^{1,17}. If culture is positive then morphological and molecular testing is performed to identify the pathogen¹ (FIG. 27—right, top panel). However, it has been shown that correct initial choice of antibiotics therapy, specifically within 1-3 hours from the initial symptom-based sepsis recognition, has a higher contribution to reducing mortality than any other medical interventionis²¹. Specifically, a 5-fold reduction in survival has been shown due to inappropriate antimicrobial therapy within first 6 hours of recognition¹². Apart from the long time to result, blood culture also suffers from other well documented problems such as sub-optimal sensitivity²², failure to identify slow growing pathogens, and substantial delay or failure to identify pathogens in BSIs for patients who have previously received antibiotics²³. Pathogen detection from blood culture is worse in neonatal patient population due to the limited sample volume (1 mL) with detection only possible in 10-15% of symptomatic neonates after excluding contaminants^{24,25}. Given these limitations and challenges of blood culture, there is a need to develop analytical and molecular diagnostic approaches with faster time to result and better sensitivity.

Currently in the U.S, nearly all U.S Food and Drug Administration (FDA) approved sepsis molecular diagnostic platforms require blood culture as a first step and thus do not effectively improve patient management²⁶⁻²⁹ (FIG. 27 top panel). Commercial kits performing NAAT typically perform a separate, upstream purification step in which DNA and RNA from crude samples are extracted and purified using solid phase extraction columns made from silica³⁰⁻³². Although the adsorption strength and capacity of these silica columns have been well characterized in previous works, most studies were conducted within the confines of high DNA loads, where the total input DNA exceeded 1 μg³³. In clinical applications, workflow typically involves treating a biological sample (blood, urine, cerebral spinal fluid, etc.) with lysis buffer to release the nucleic acids from cells, bacteria, and/or virions. The DNA is then isolated from solution using a solid phase extraction column, retrieved using an elution buffer, and quantified via molecular tests for diagnosis³³. This nucleic acid purification has two inherent loss mechanisms. First, DNA adsorption onto the column may be inefficient, and second, the purified DNA may not be efficiently eluted from the column³³. Hence, when processing ˜1 mL of blood, currently available nucleic acid purification kits cannot efficiently capture and retain a few copies of target pathogenic DNA against a vast background of contaminants and millions of copies of Human genomic DNA. This is also the reason why the FDA approved Sepsis NAATs such as Biofire's FilmArray and Nanosphere's Verigene can only test for pathogens after a positive blood culture^{34,35}. Of the few tests that circumvent the need for blood culture, most use conventional techniques such as polymerase chain reaction on purified DNA from whole blood or serum. Consequently, these tests suffer from low and variable sensitivity between 13-100% and overall lower detection limit due to combined inefficiency of nucleic acid extraction with inefficiencies of downstream test specific processes such as splitting the extracted DNA into multiple reactions^{1,36-39}.

Currently, there is only 1 FDA approved pathogen identification platform from T2 Biosystems for blood stream infections. The T2 Biosystems' bacteria and candida panels can identify specific pathogens from whole blood samples in 5.4 hours (mean time)²², where the T2 bacteria panel was approved in 2018⁴⁰. Although this technique bypasses the need for conventional DNA purification by using a proprietary mutant polymerase for performing pathogen specific PCR from whole blood lysate, it uses expensive reagents and instruments such as magnetic nanoparticles and magnetic resonance reader along with the thermocycler for PCR, as visual readouts are impaired due to high heme background in the lysate. This increases the cost per assay and prevents possible translation into the low and middle-income countries. Moreover, the detection limit for E. Coli for T2 bacteria panel is 11 cfu/mL which falls short of the required sensitivity especially for neonatal patients where studies have shown that concentrations in 68% of culture-positive cases fall well below 10 cfu/mL^{41,42}.

To address the above challenges, we have taken a materials approach to whole blood processing which minimizes sample preparation and simultaneously offers unprecedented sensitivity. Here, we introduce a blood-processing module where we create a porous micro- and nano-fluidic network within dried blood matrix (FIG. 27 middle, left (panels labeled c-e), allowing the polymerase to access the DNA inside the blood matrix and initiate the amplification (FIG. 27 middle, right (panels labeled f-g)). Previous studies have tried extracting viral nucleic acids using conventional purification techniques from dried blood spots on filter paper (limited to 50 μL blood) but have showed amplification with limited sensitivity⁴³⁻⁴⁵. Our continuum scale simulation studies revealed that for low cfu pathogen counts, the ideal approach is to introduce the enzymes into the dried blood matrix through diffusion in micro- and nano-fluidic network instead of trying to elute the target DNA out of the blood matrix. In our platform, the dried blood does not take part in the reaction and acts as a substrate through the duration of the reaction where the inhibitory elements such as platelets, cells, and proteins are neutralized and become a part of the substrate. We show that the generated porosity and the micro- and nano-fluidic network allow for enzymes to access DNA in the liquid phase and initiate amplification with single molecule sensitivity inside the dried blood matrix thus bypassing the need for conventional DNA purification⁴⁶ (FIG. 27 bottom right (panel labeled g)). The drying of blood can be accomplished in as low as 10 minutes at high temperatures (95° C.), significantly reducing the sample preparation time. Moreover, the dried blood solid phase does not remix with the supernatant and keeps the high heme locked in the background in red blood cells, whereas the fluorescent amplicons post-amplification are concentrated in the clear supernatant phase, giving an extraordinary signal-to-noise and fluorescence change even with more than 20% blood per reaction volume (FIG. 27 middle right (panel labeled h)). Hence, the term “biphasic amplification” for the reactions (FIG. 27 middle right (panels labeled f-h)). We couple this biphasic blood processing module with a robust Bst polymerase, which we have previously shown to perform amplification in tissue matrices and loop mediated isothermal amplification (LAMP) reaction, minimizing the need for a thermocycler⁴⁷.

We first demonstrate our platform by efficiently amplifying cell free methicillin resistant S. aureus (MRSA) and E. coli DNA in microliters of dried whole blood with single molecule sensitivity (1 copy/4 μL blood). We then couple our blood processing module with mechanical bead lysis (FIG. 27 middle left (panel labelled d “bacteria mechanical lysis”)) to demonstrate a detection limit of 1.2 cfu/mL for MRSA (gram +ve), methicillin sensitive S. aureus (MSSA, gram +ve), E. coli (gram −ve), and Candida Albicans (Fungus) pathogens from 0.8 mL of spiked healthy human blood samples. We test 170 spiked samples, with 80 samples having concentration below 10 cfu/mL. The reliability of the developed approach is further confirmed by testing 63 whole blood clinical samples, including 14 positives for E. coli and 1 positive for MSSA (100% sensitivity and 100% specificity). The reliability of the developed approach is further confirmed by testing 63 clinical whole blood samples, including 14 E. coli positive, 1 MSSA positive, and 15 culture negative samples (100% sensitivity and 100% specificity). In addition, 40 samples culture-positive for organisms other than MSSA, MRSA, E. coli, or Candida are also tested as specificity controls. The sample-to-answer time of our platform is less than 2.5 hours. It is important to note that for E. coli, the platform provided herein is almost an order of magnitude more sensitive than the current only FDA approved culture-free bacteria panel²².

Assay Design for Cell-Free DNA in Blood:

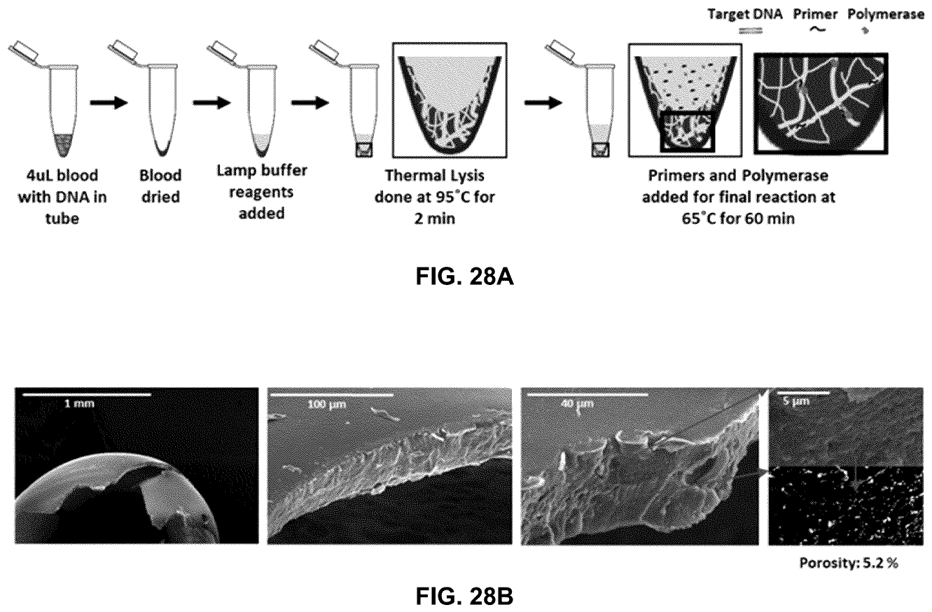

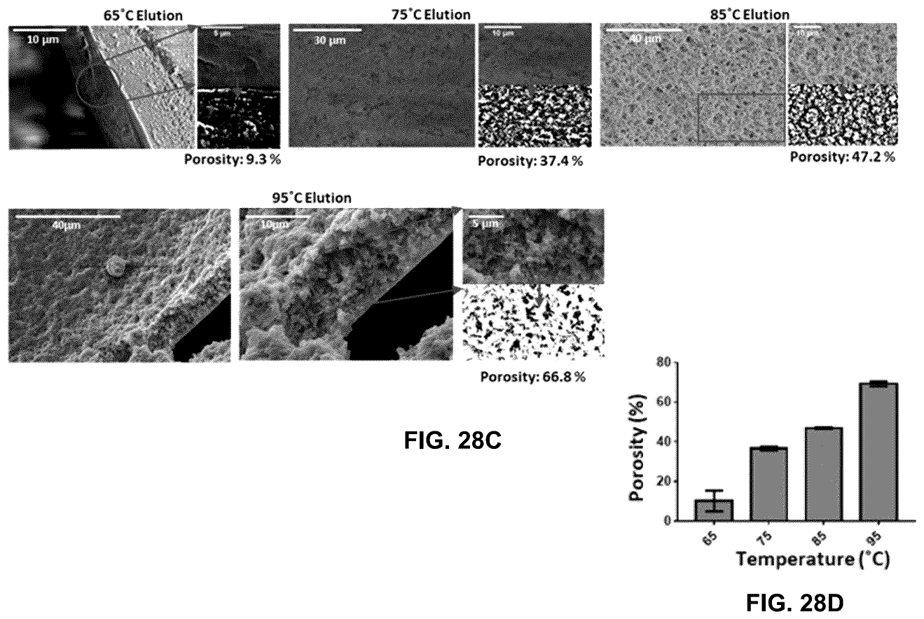

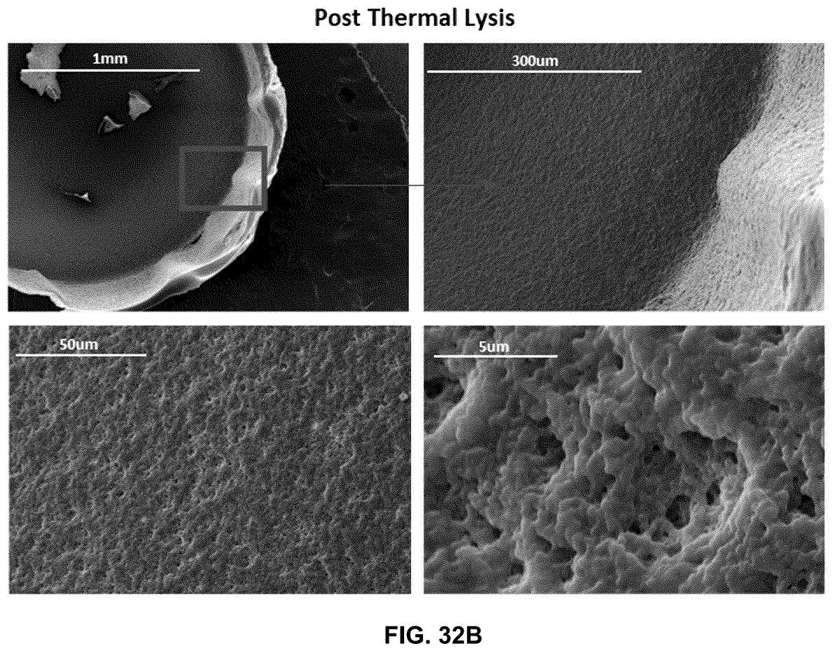

We first design the biphasic amplification process using spiked DNA in whole blood, akin to cell-free DNA in whole blood in small blood volume reactions by adding 4 μL of whole blood with spiked pathogen DNA into standard 0.2 mL PCR tubes, followed by rapid drying of the blood in a heater (37° C., 20 min) (FIG. 28A). This protocol represents the same process in FIG. 27. SEM images show that after drying, the blood becomes a solid substrate/sheet with a porosity at or below 6.4% (FIG. 28B and FIGS. 32A-32B). In the next step, we add the amplification buffer and reagents (without primers and polymerase), and subsequently generate a porous physical network inside the dried blood matrix by performing a wet thermal lysis for 95° C. for 2 minutes. The SEM characterization of dried blood after thermal lysis at different temperatures from 65° C. to 95° C. show micro-nanoscale pores and networks. Image analyses show an increase in porosity of the dried blood matrix from ˜10% at the 65° C. lysis temperature to over 60% for 95° C. (FIGS. 28C-28D). Hence, 95° C. is selected as the final thermal lysis temperature. Physical micro- & nano-fluidic networks are observed inside the dried blood matrix after the thermal lysis step (FIG. 28C and FIGS. 32A-32B).

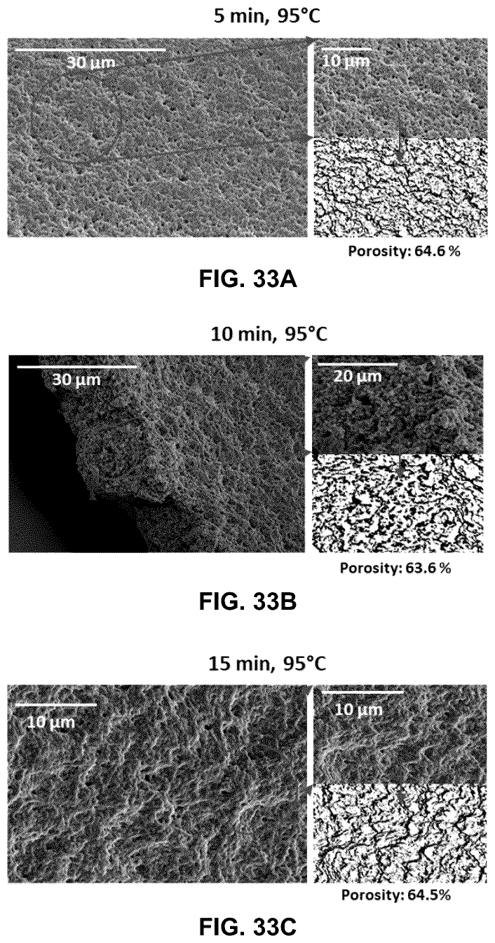

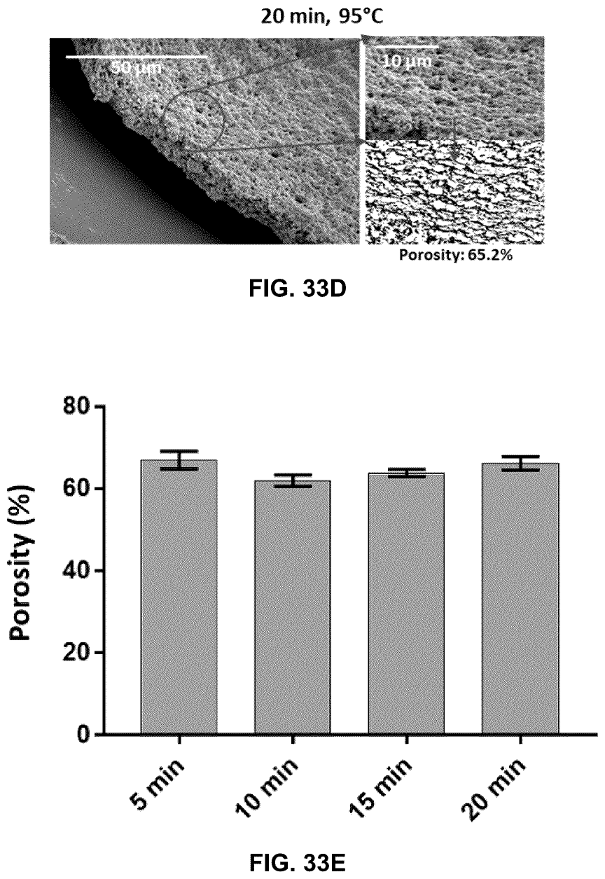

In addition to varying the blood drying conditions and thermal lysis times for porous network generation discussed above, we also explore the effect of increased thermal lysis times on the porosity of the dried matrix. To maximize porosity, we tried longer times of thermal lysis at 95° C. from 5 minutes to up to 20 minutes and the SEM analysis of porosity is shown in (FIGS. 33A-33E). As can be seen from the analysis, for whole blood, increased thermal lysis times gave similar porosity results (ranging between 63.6% and 65.2%) in comparison to previously observed with thermal lysis at 95° C. for 2 minutes. Hence for our final protocol we select 2 minutes of thermal lysis at 95° C.

In the final step, the primers and polymerase are added, and the loop mediated isothermal amplification (LAMP) reaction is performed at a constant temperature of 65° C. for 60 minutes. The micro- and nano-fluidic network inside the dried blood sample allows primers and polymerase to access the DNA molecule via diffusion and initiate the amplification reaction inside the blood matrix. It is important to note that we use Bst polymerase for our biphasic reactions which we have previously shown to be robust against tissue matrices⁴⁷. The solid dried blood phase also allows for a clear supernatant phase where high signal to noise and a large fluorescence change can be observed during amplification, comparable to that of purified DNA reactions with no blood (FIG. 27). Since the optical reading components are located on the top of the reaction tube in the QuantStudio 3 system that we used, the solid dried blood at the bottom of the tube does not significantly interfere with the fluorescence reading from the fluid above (“supernatant” fluid) the solid phase. Numerical simulation and experimental validation of biphasic reaction mechanism is provided in the Results 1 section below.

Detection of MRSA and E. coli Cell Free DNA in Whole Blood in Biphasic Format

To evaluate the range and limit of detection of our biphasic assay for cell free DNA, we next spiked serial dilutions of MRSA and E. coli DNA in whole blood. For MRSA, we amplified the mecA gene which is responsible for the methicillin drug resistance. For E. coli, using previously published LAMP primers^{48,49}, we amplified the malB gene which is conserved in majority of infectious E. coli strains.

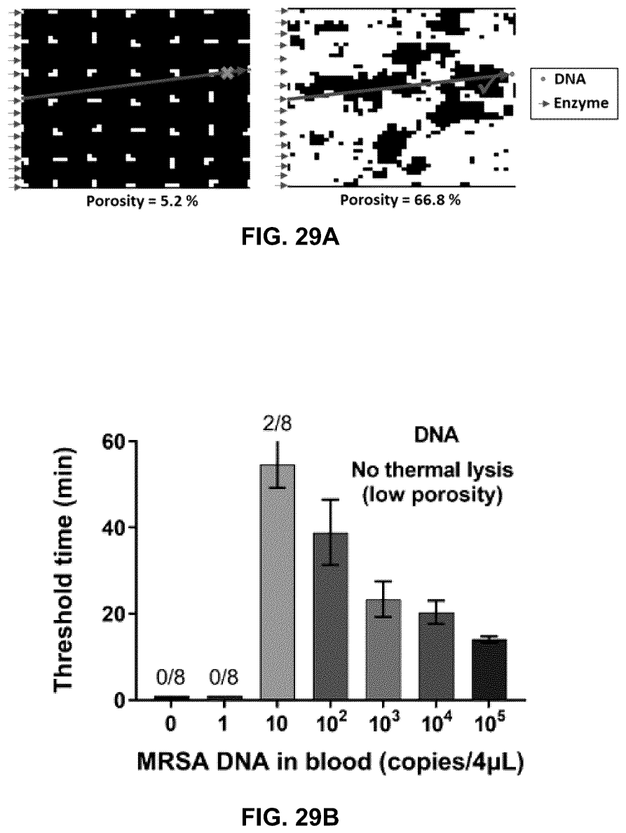

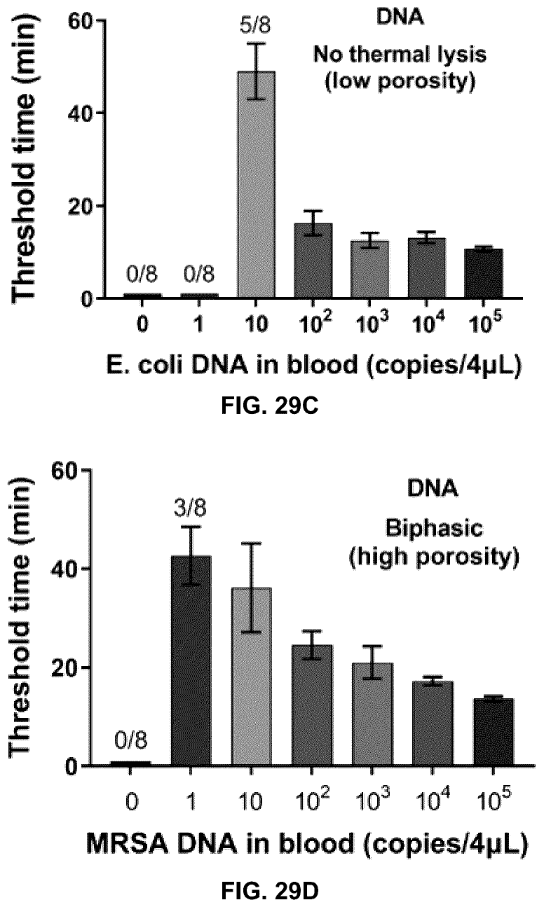

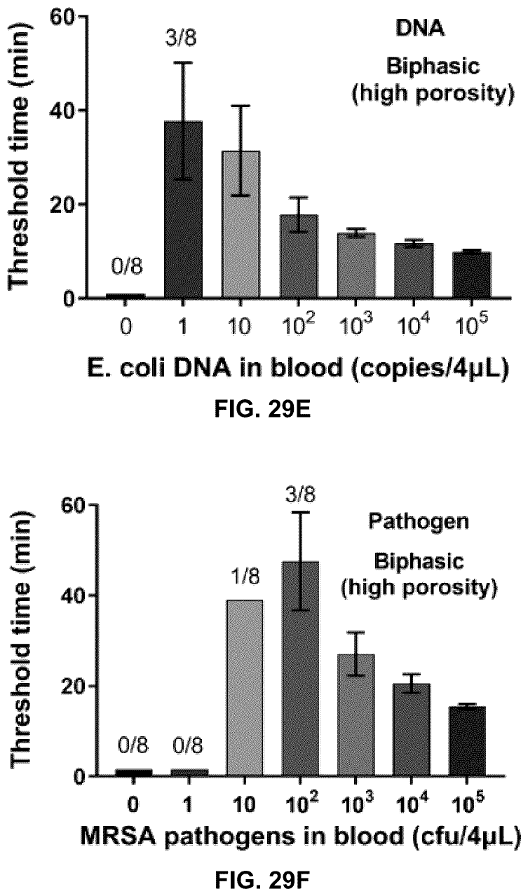

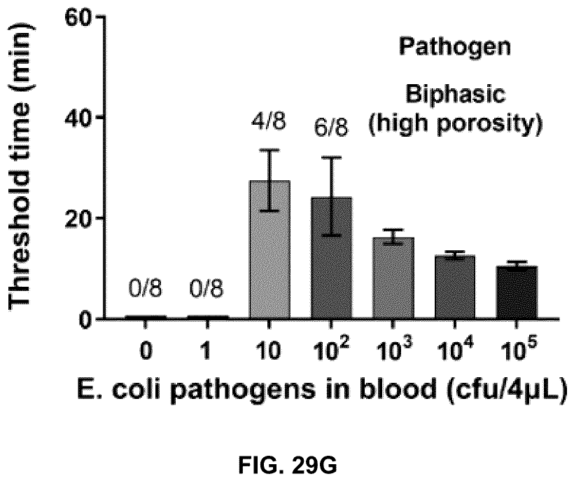

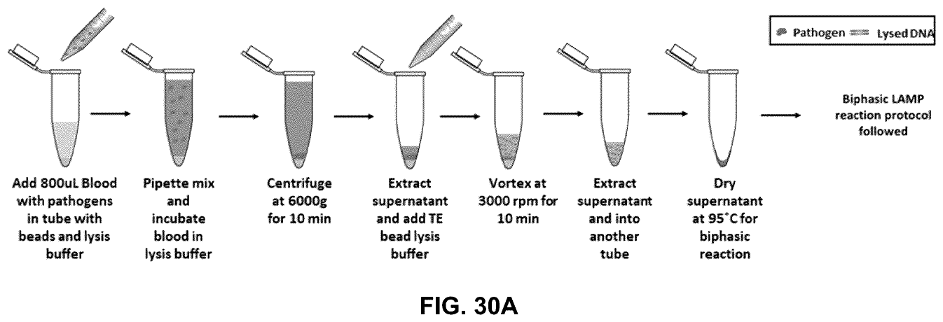

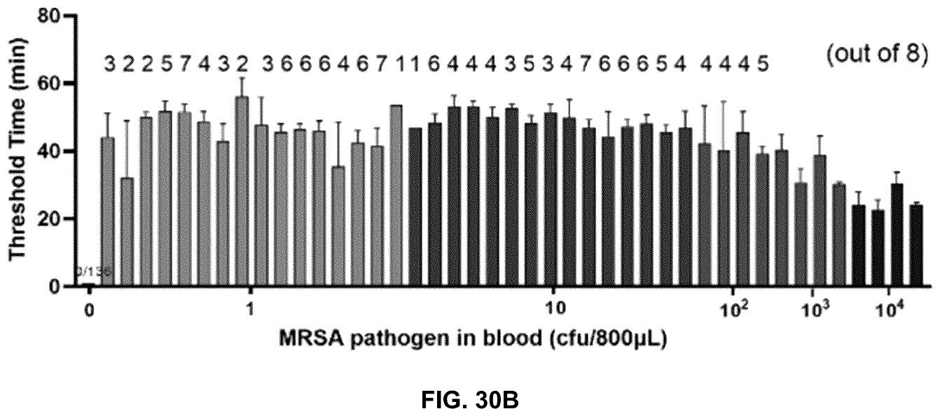

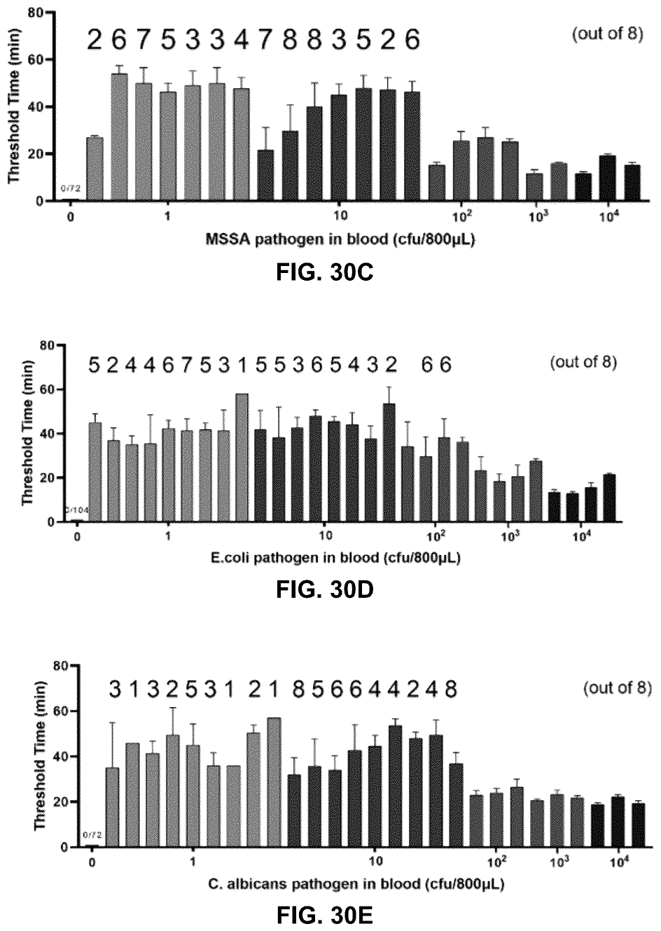

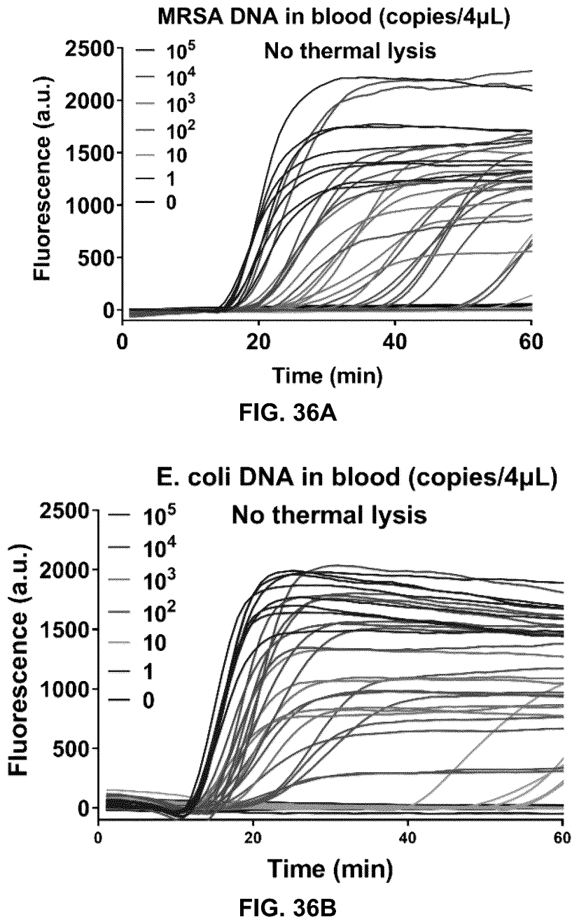

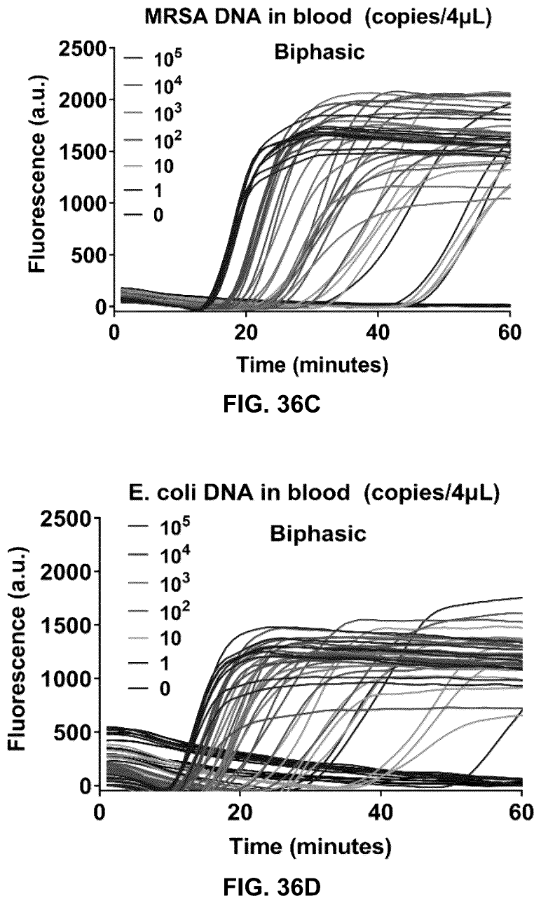

First, to experimentally examine if simple drying step and dried blood matrix provides enough sensitivity for the detection of cell free DNA, we performed no thermal lysis controls. The porosity simulation (FIG. 29A) showed that the enzyme could not diffuse to the target DNA inside the blood matrix due to low porosity (˜5%). The amplification curves, and the threshold time bar graphs in no thermal lysis control reactions from blood are shown for MRSA DNA (FIG. 36A and FIG. 29B) and E. coli DNA (FIG. 36B and FIG. 29C). As predicted by porosity simulation, the detection limit for both the reactions was found to be 100 copies/4 μL of blood, highlighting the need for thermal lysis. Comparatively, it is shown that extra thermal lysis step and consequent high porosity in biphasic reactions allows the enzyme to reach the DNA inside the blood matrix (FIG. 29A). The amplification threshold times in blood using our biphasic format are shown for MRSA DNA (FIG. 29D) and E. coli DNA (FIG. 29E). The amplification curves are shown in the FIGS. 36C-36D. The limit of detection for both the cases was found to be 1 copy/4 μL of whole blood (Limit of Detection is 1 copy since amplification frequency=expected sampling frequency) showing single molecule sensitivity in our biphasic reactions. It is important to note that the created micro- & nano-fluidic network allows access to even a single copy of DNA inside the solid blood matrix phase in our protocol. As expected, a larger range of amplification threshold times (10-20 minutes) was observed for low DNA copy number amplifications. Additional characterization of biphasic reactions with cell free DNA can be found in the Results 2 section below. This provides evidence that the instant platform is useful for detecting cell-free nucleic acid, including DNA.

Detection of Low Cfu Pathogens in Whole Blood in Biphasic Format

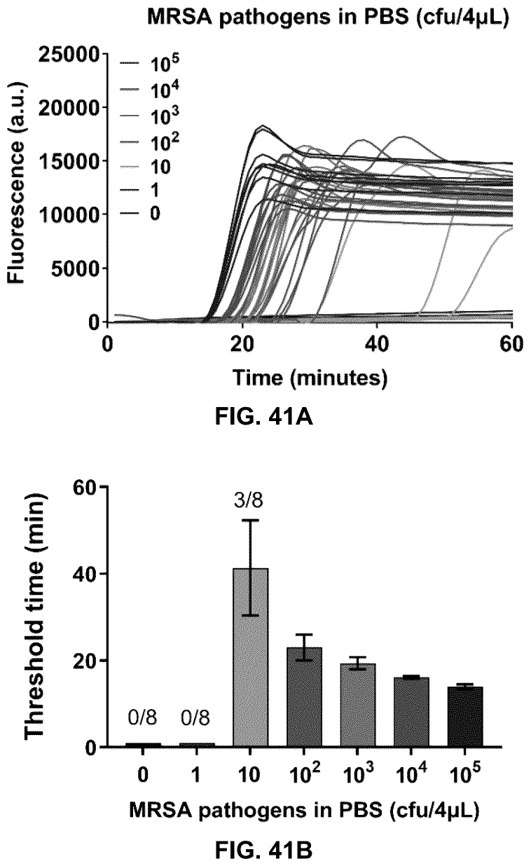

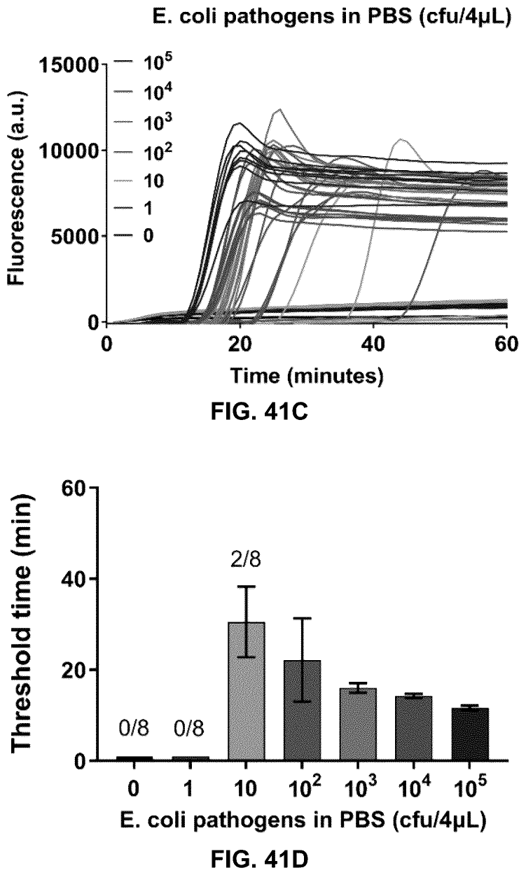

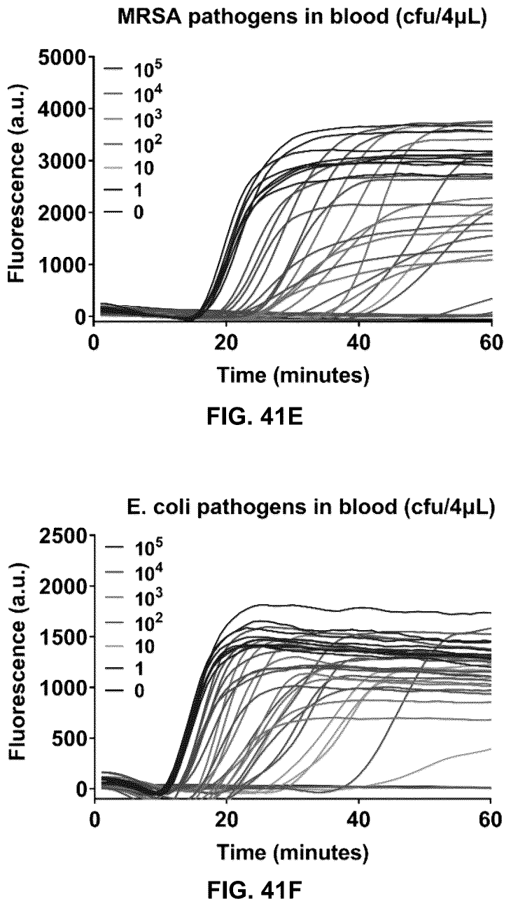

Next, to translate our blood processing and biphasic reaction module to detect pathogens in blood, we first carried out buffer reactions with pathogens spiked in PBS instead of blood. The amplification curves and the threshold times for MRSA (FIGS. 41A-41B) and E. coli (FIGS. 41C-41D) pathogens are shown. The limit of detection for both the pathogens was found to be 100 cfu with only 3/8 (MRSA) and 2/8 (E. coli) replicates giving amplification for 10 cfu. This reduced sensitivity is expected, as thermal lysis (95° C., 2 min), performed to disrupt the bacterial cell wall (see methods section for detailed protocol) has been previously shown to be inefficient in lysing bacteria^{50,51}. Next, we repeated the above experiments with pathogens in blood in the biphasic reaction format for MRSA (FIG. 41E and FIG. 29F) and E. coli (FIG. 41F and FIG. 29G). Similar reduced limit of detection of 1000 cfu was observed for both the pathogens with only 3/8 (MRSA) and 6/8 (E. coli) replicates giving amplification for 100 cfu. These results highlight the need for coupling of our biphasic technique with a more efficient mechanical bacterial cell lysis approach to allow access to DNA and achieve improved sensitivity of our assay for detection of pathogens at low concentrations relevant to BSI and sepsis⁵²⁻⁵⁵. However, it is important to note that the optimized biphasic approach with small volumes of whole blood and moderate LODs is important in itself, for example, for finger pricks or heel lance nucleic acid testing in newborn blood samples⁵⁶.

Assay Design for ˜1 Cfu/mL Limit of Detection of Bacteria in Whole Blood

To address the challenges in pathogen identification in blood stream infections, specifically in sepsis where the pathogen concentrations can often be below 10 cfu/mL^{41,42}, we couple the biphasic blood processing and reaction module with conventional bead-based mechanical pathogen lysis. FIG. 30A is a protocol where 800 μL of whole blood with pathogens is loaded into a 2 mL tube containing hypotonic red blood cell (RBC) lysis buffer and 100 μm glass beads. The blood is mixed with the RBC lysis buffer to lyse majority of the red blood cells and centrifuged thereafter to pellet the intact cells. After discarding the supernatant from the RBC lysis, TE buffer is added, and mechanical bead lysis is performed by vortexing at 3000 rpm for 10 minutes. Note that, any cell free DNA will also be discarded with the supernatant in the above step and only intact cells will be retained. The blood lysate post mechanical bead lysis from a single sample is aliquoted into 8 standard 0.2 mL PCR tubes with 30 μL per tube and dried for the biphasic amplification. Sample is considered positive for the target if any of these 8 tubes (from the same starting sample) show amplification. The drying is performed by heating the sample at 95° C. for 10 minutes, followed by the LAMP reaction protocol for biphasic format (methods section).

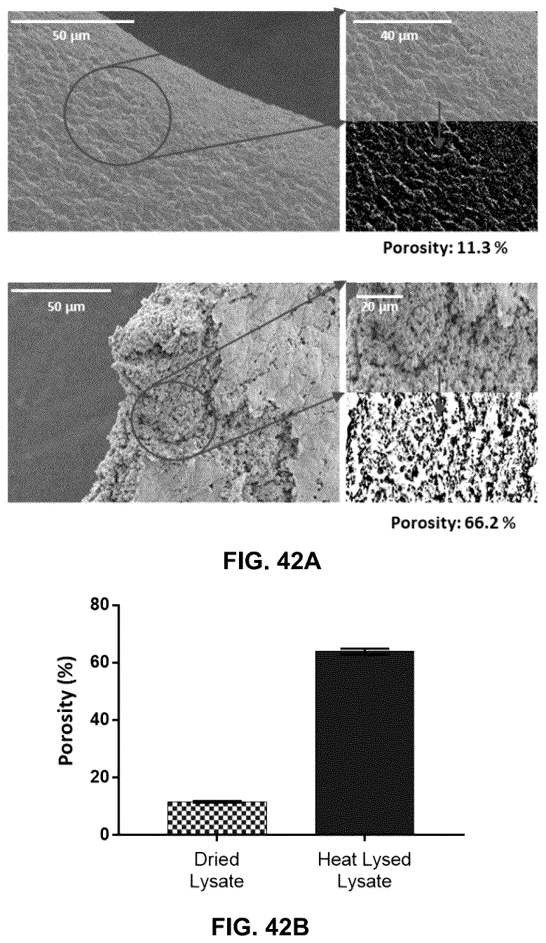

To characterize the microenvironment, we perform SEM analysis of dried blood lysate after bead beating and found the porosity of the dried matrix pre and post thermal lysis to be 11.5% and 63.8%, respectively, which is very similar to what we previously observed without bead beating. The SEM analysis can be found in supplementary information (FIGS. 42A-42B). It is important to note that, for high volume (0.8-1 mL) blood processing, we were able to rapidly dry 30 μL of blood lysate post bead beating at 95° C. while retaining the higher porosity post thermal lysis (˜ 63.8%). This is likely because the clotting proteins and factors were removed along with the supernatant during the RBC lysis steps while intact cells and pathogens were sedimented during centrifugation (6000 g, 10 min).

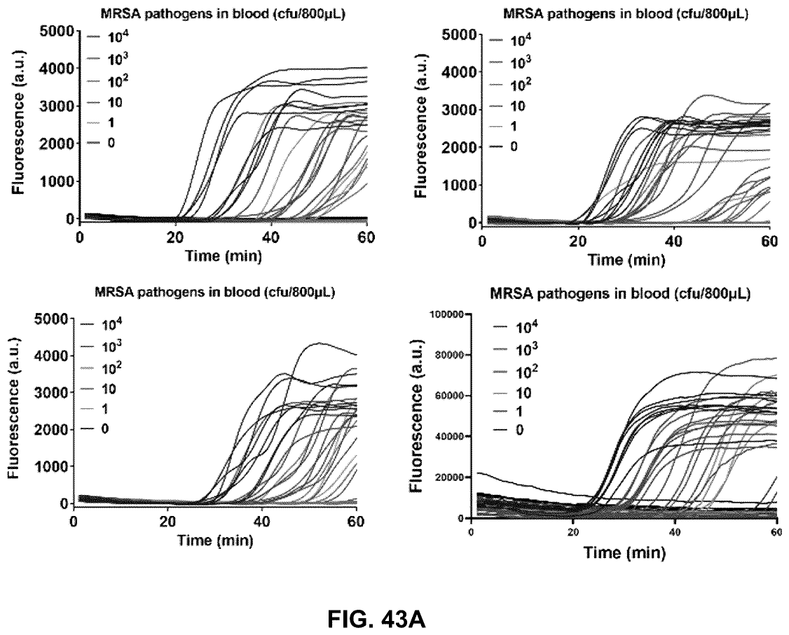

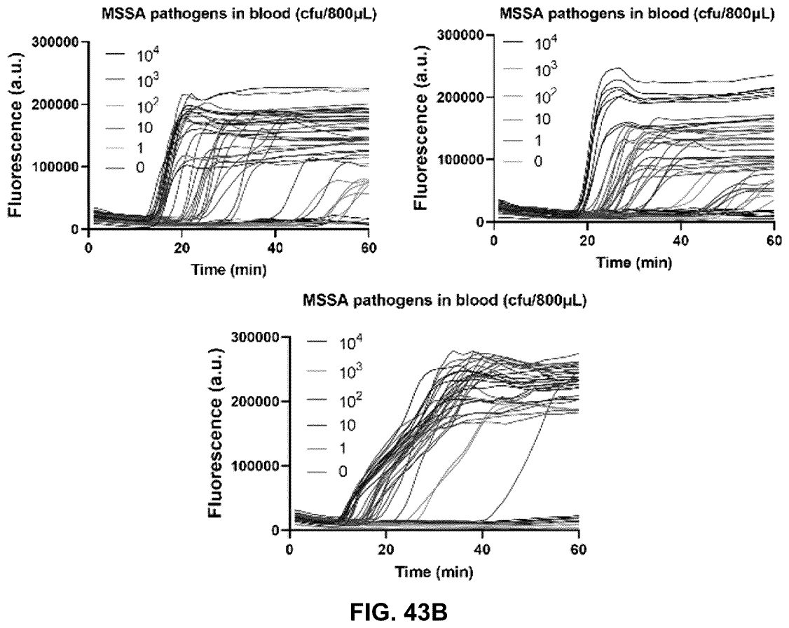

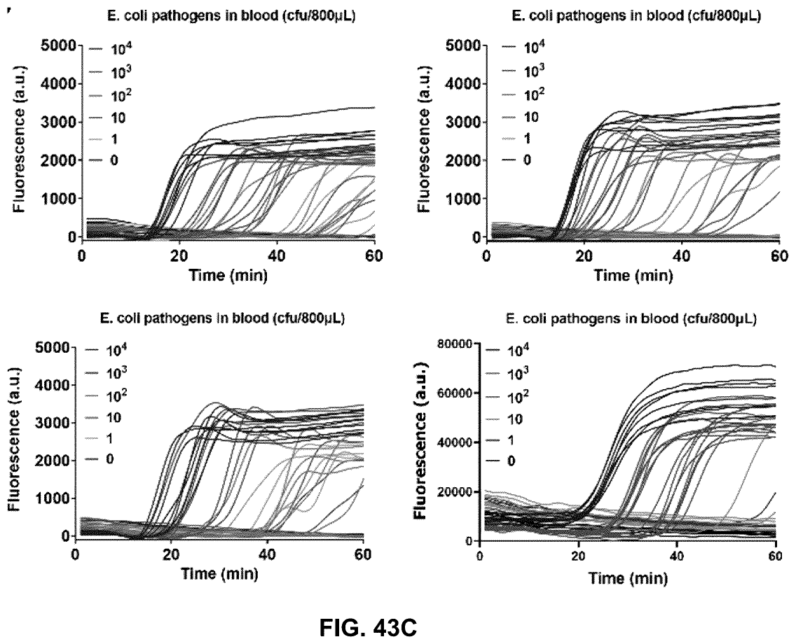

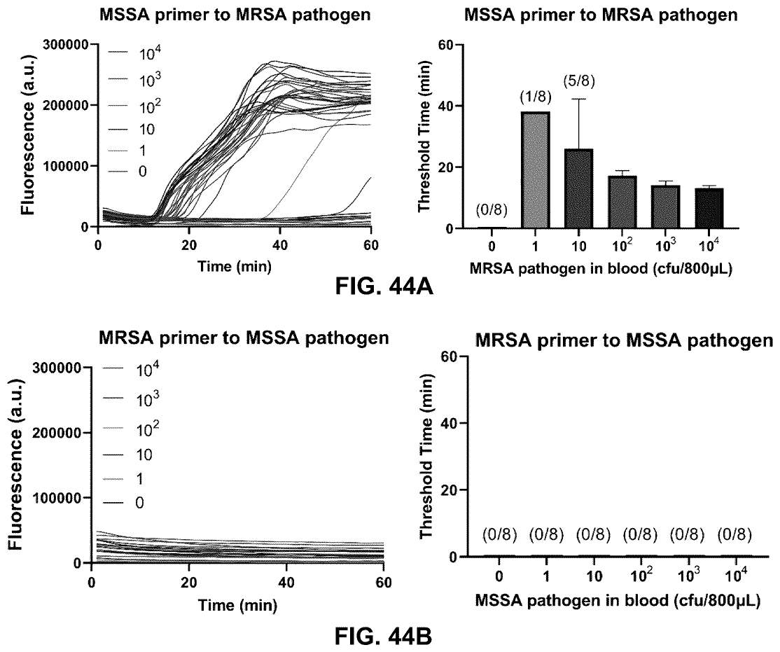

The threshold time bar graphs for MRSA (FIG. 30B), MSSA (FIG. 30C) and E. coli (FIG. 30D) spiked in 800 μL of whole blood is shown. The amplification curves MRSA, MSSA (fem A gene) and E. coli are shown in FIGS. 44A-44B⁴⁸. It is important to note that the concentration range of the assay (1.2e4 to 1.2 cfu/mL) was chosen to overlap with the reported pathogen concentration in patients with blood stream infections⁵²⁻⁵⁵. Moreover, MRSA, MSSA and E. coli serve as good targets to demonstrate our platform, not only because MRSA and MSSA are gram positive (thicker cell wall) whereas E. coli is gram negative, thus covering a range of bacterial infectious pathogens, but also because they have among the highest disease burden of all BSI pathogens⁵⁷. Overall, the detection of MRSA, MSSA and E. co/i was performed from 134 mock samples where 62 samples were at 10 cfu or 1 cfu per 800 μL of whole blood and 39 were negative control samples (FIGS. 30A-30E and FIGS. 43A-43C). The limit of detection of our MRSA, MSSA and E. coli assays in our platform was found to be 1.2 cfu/mL. While many more replicates need to be performed, we clearly show an improvement of an order of magnitude over the current state of the art E. coli detection limit of 11 cfu/mL in the only FDA approved blood culture free diagnostic platform²². Moreover, to confirm that our primers can distinguish between MRSA and MSSA, specificity tests were performed for MSSA primers using MRSA pathogens (FIG. 44A) and MRSA primers using MSSA pathogens (FIG. 44B). As a result, by using the MRSA and MSSA primers, the presence or absence of fem A- and mec A-gene (resistance gene) was identified and therefore MRSA and MSSA could be distinguished using our platform. Control experiments and analysis of heme content for biphasic reaction can be found in Results 3 below.

Assay Design for ˜1 Cfu/mL Limit of Detection of Fungal Pathogens in Whole Blood

To show that our platform applies to a broader pathogen range, we evaluate the limit of detection of fungal pathogens using mechanical bead lysis coupled with biphasic blood processing. Candidemia is a high mortality (40%) fungal blood stream infection caused by the Candida species of fungus where rapid diagnosis is crucial^{12,13,58}. Studies have shown that initiation of correct antifungal treatment in less than 12 hours can reduce mortality from 40% to 11%^{13,53}. However, its current clinical gold standard of diagnosis is blood culture which takes 2-5 days for culture growth and has a low sensitivity of ˜-50%⁵⁹. Within Candida species, we chose to detect Candida Albicans in our platform since it is one of the most prevalent and is responsible for invasive candidiasis in majority of the cases in the United States⁶⁰.

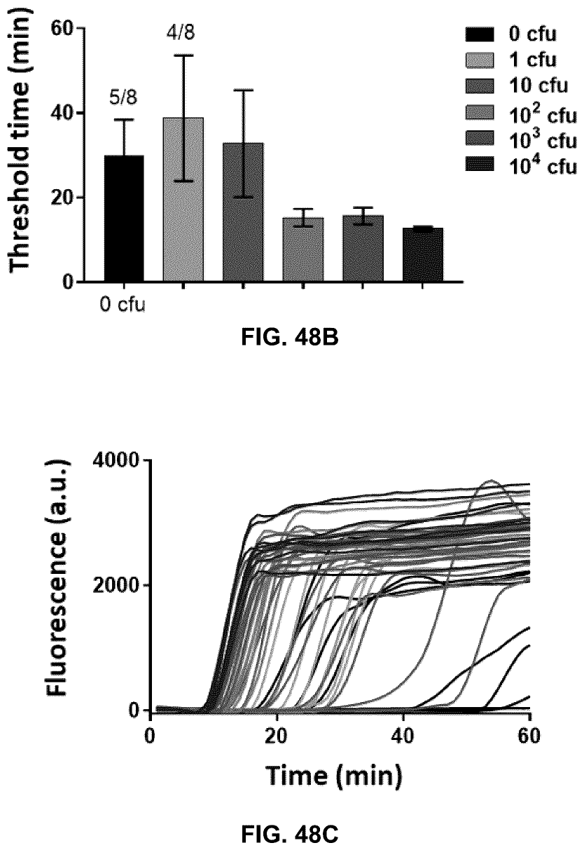

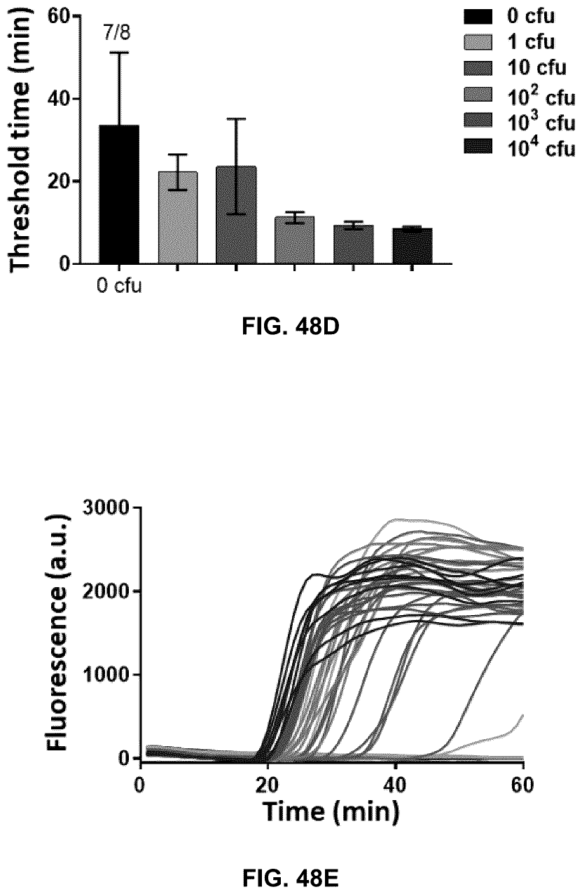

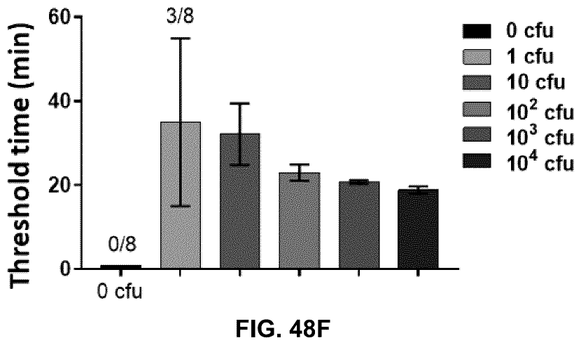

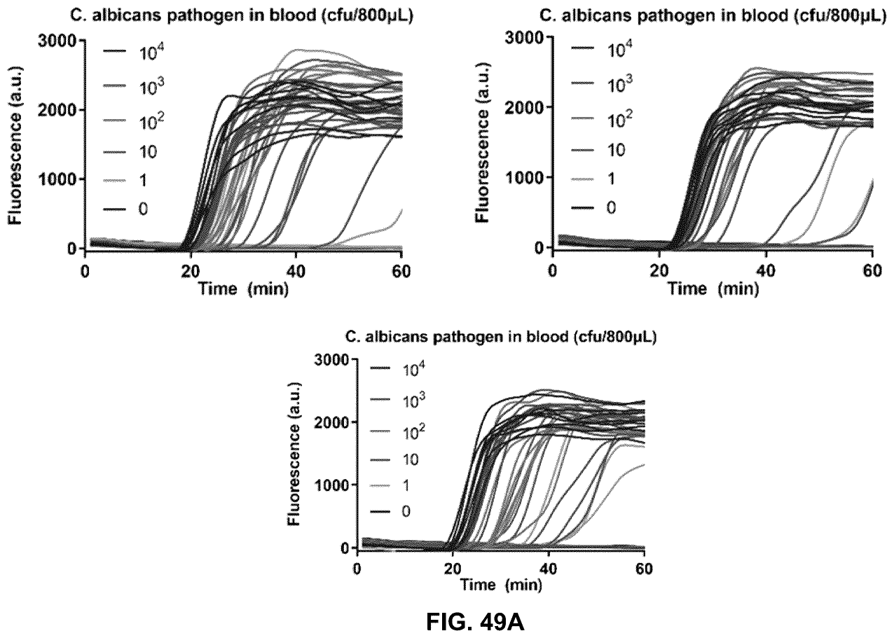

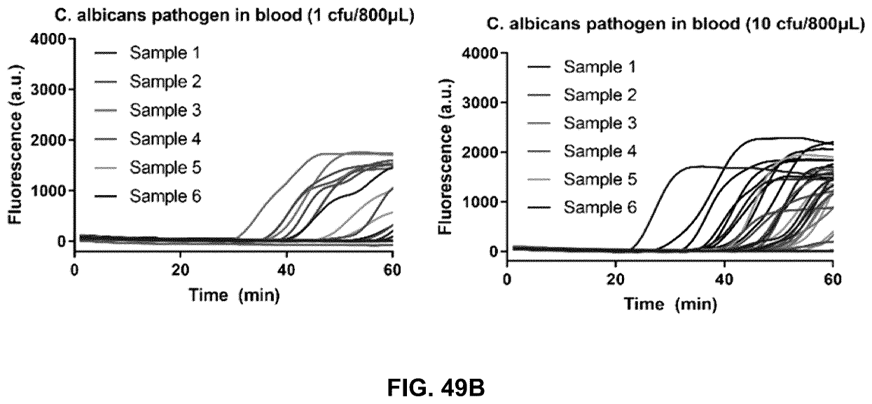

In comparison to bacteria, fungus are larger in size (10-12 μm) and their cell wall composition does not include peptidoglycan and lipid layers, but instead includes layers of complex polysaccharides including chitin, β-1,3-glucans, and β-1,6-glucans with cell wall proteins covalently bonded to this network^{61,62}. This makes the fungal cell wall mechanically very strong and difficult to break. To disrupt the fungal cell wall, we modified our mechanical bead lysis to include larger 500 μm diameter glass beads⁶³ while the rest of the protocol remained the same. For the LAMP reaction, we targeted the intervening transcribed spacer 2 (ITS2) region within the Candida rDNA using previously published LAMP primers⁶⁴. We first optimized the reaction temperature and primer concentrations for our 800 μL high blood volume biphasic format using the above primers. We found that a higher reaction temperature of 67° C. along with reduced primer concentrations of 0.04 μM of F3 and B3, 0.33 μM of FIP and BIP, and 0.17 μM of LF and LB primers yielded the best results with no non-specific amplification (FIGS. 48A-48F). The limit of detection experiments with optimized protocol starting from 800 μl whole blood spiked with Candida Albicans are shown (FIG. 30E and FIGS. 49A-49B). We could reliably detect 1 cfu/800 μL (LOD of 1.2 cfu/mL). Together, these figures show detection of Candida Albicans from 36, 800 μL spiked whole blood samples with 18 of these being low count samples (10 cfu, 1 cfu per 800 μL blood) and 9 negative control samples.

Assay Validation for Pathogen Identification from Clinical Whole Blood Samples

Finally, we demonstrate the efficacy of our biphasic reaction to identify circulating pathogens in blood from clinical whole blood samples, using the process currently followed in clinical practice as a control. We collect a total of 724 samples, of which 63 samples (15 negative, 48 positive) are tested using our biphasic approach. The clinical samples are first analyzed using current clinical practice (blood culture and PCR) and then the obtained results are compared with our results (biphasic approach).

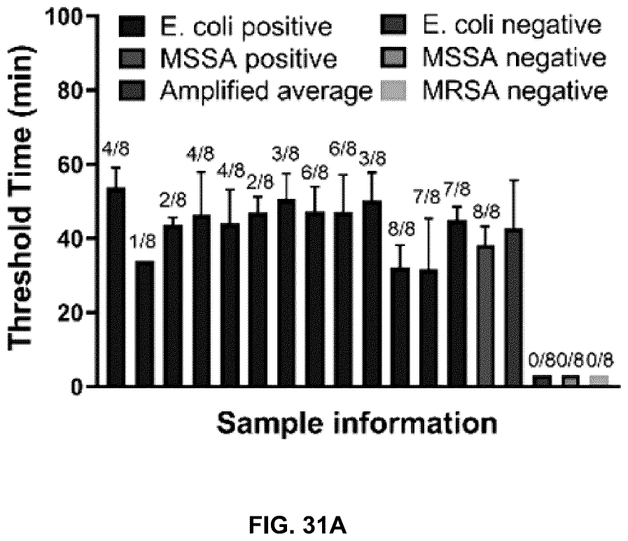

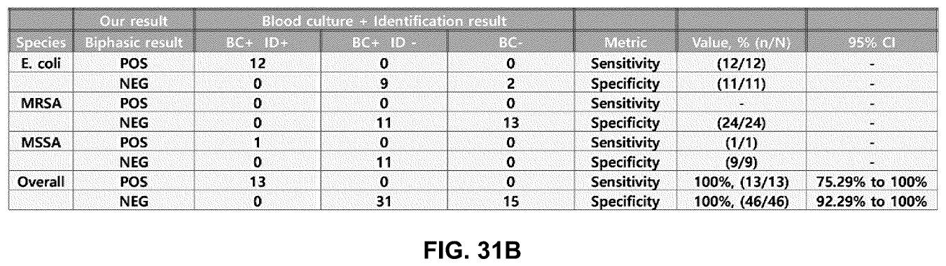

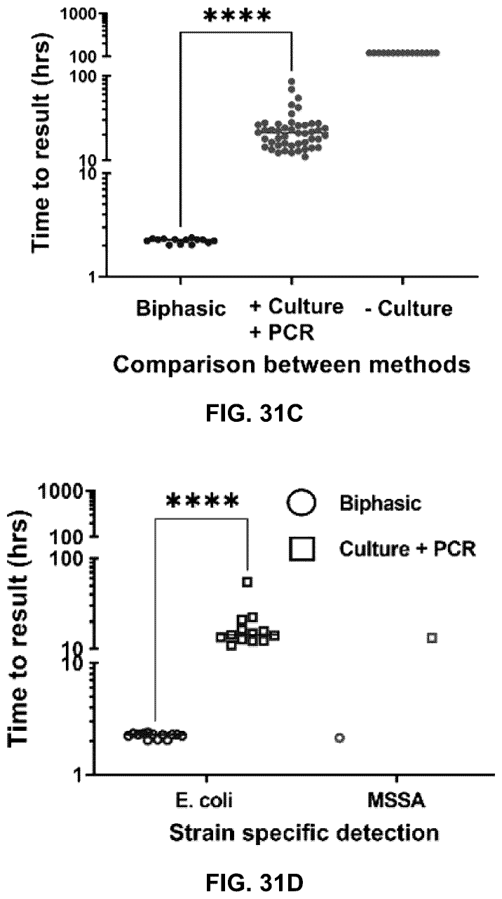

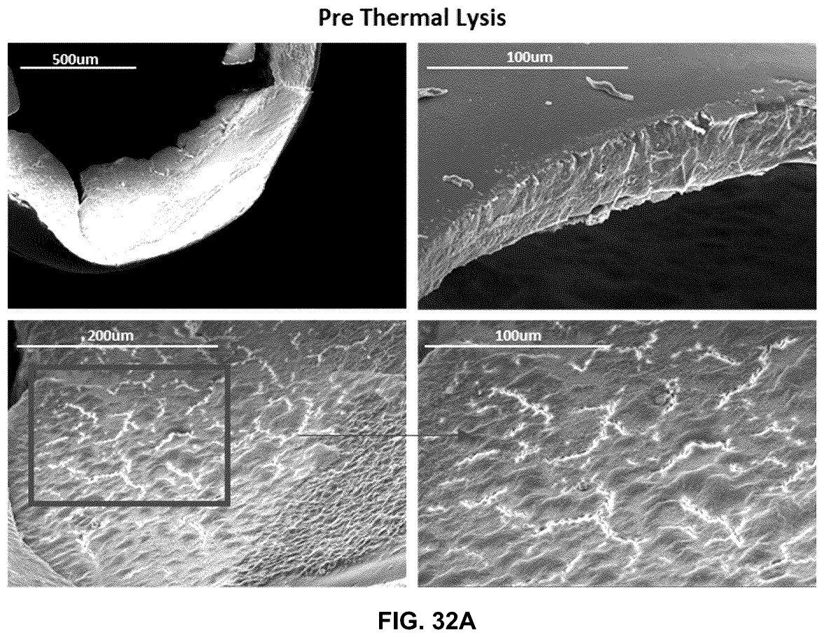

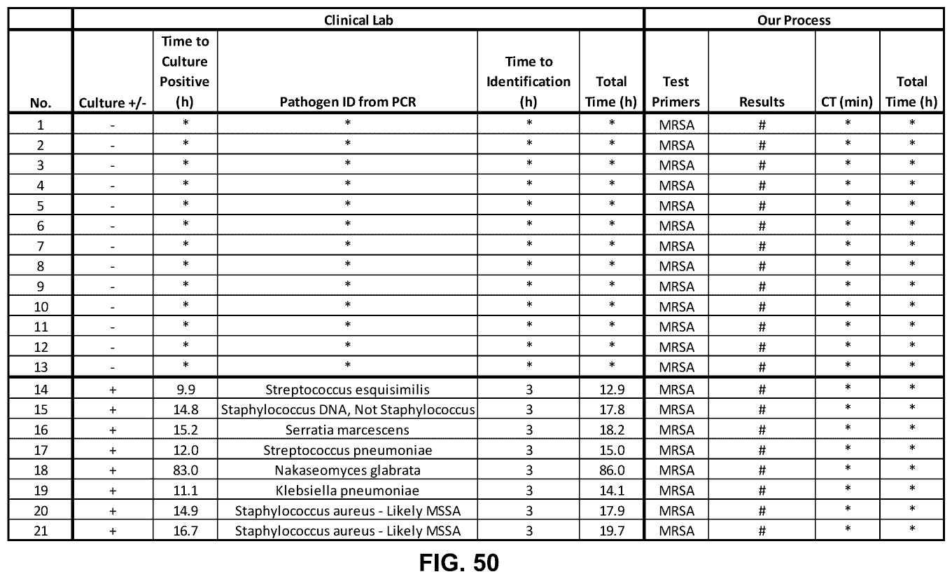

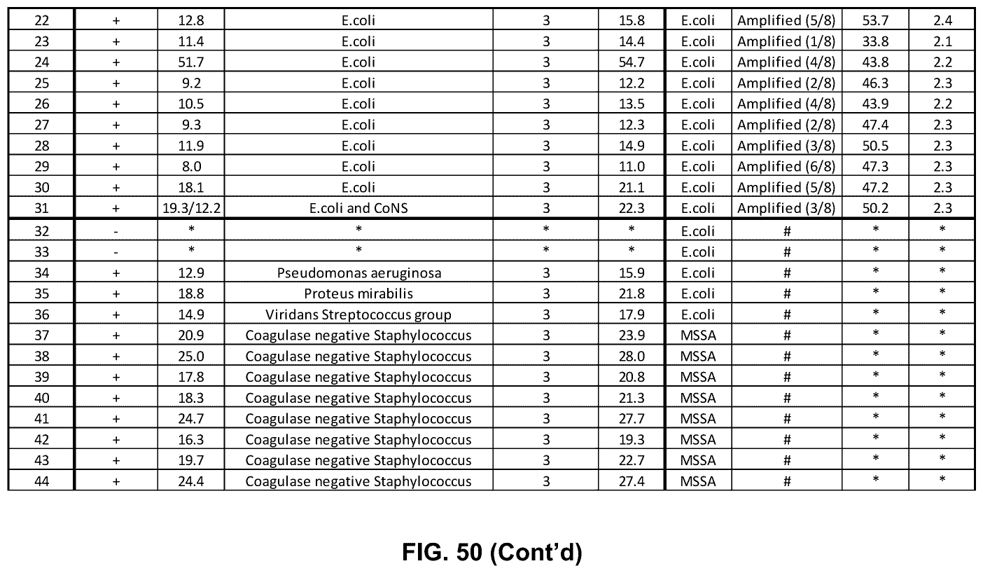

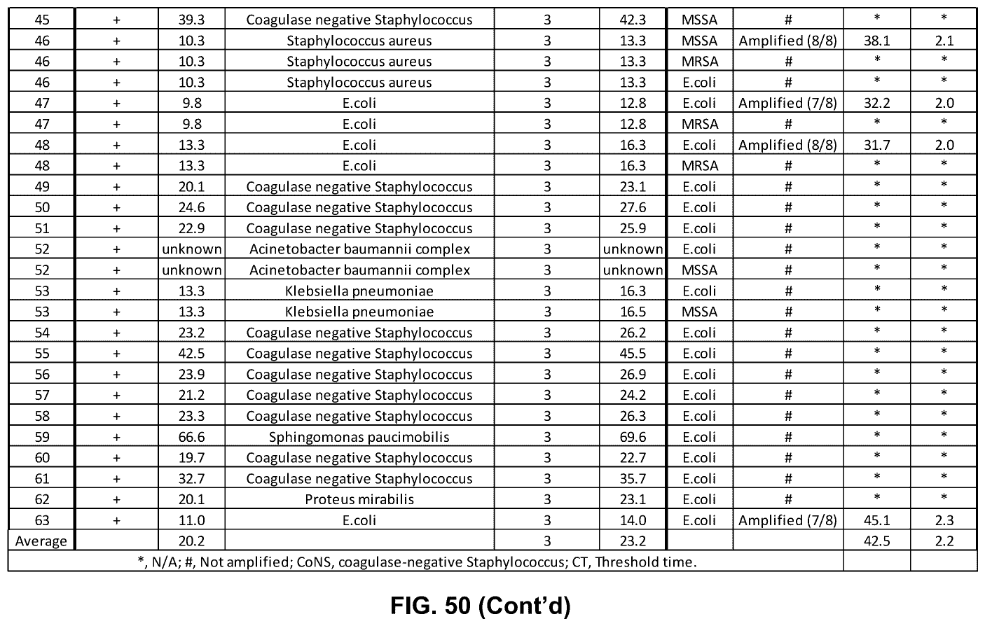

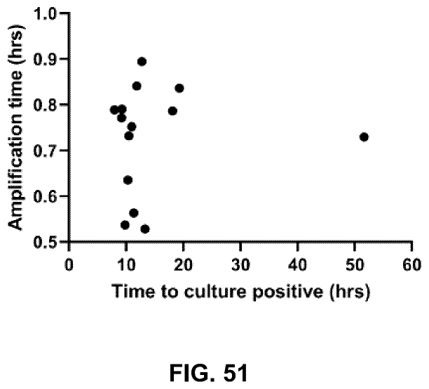

Clinical laboratory results (including culture time and identification time) are summarized along with results from our biphasic process (test primers and threshold time) in FIG. 50. To analyze the clinical samples, three primer sets (specific against E. coli, MRSA, and MSSA) were used. Of the clinical samples we tested, 14/63 samples (13 E. coli and 1 MSSA) were specific to targets that our primer sets can detect. Of these 14, 5 samples (Sample ID: 46, 47, 48, 52 and 53) were tested with more than one set of primers to confirm specific identification and assay specificity (FIG. 50). As a result, FIG. 31A demonstrates the threshold times for 14 amplified samples. The average threshold time for the 14 amplified samples was 42.5±10.1 minutes. Considering that mock samples of E. coli and MSSA (>100 cfu/mL) were amplified within 40 minutes (FIGS. 30C-30D), it can be inferred that most of the samples analyzed were <100 cfu/mL. On the other hand, no amplification was observed in the analysis of negative samples (15) nor during the analysis of positive samples (40) for organisms other than E. coli, MSSA, and MRSA. FIG. 31B summarizes the sensitivity and specificity of our assay. Our assay correctly identified all samples positive for E. coli and MSSA and identified all samples negative or positive for other organisms as negative for E. coli, MSSA, and MRSA, resulting in a sensitivity and specificity of 100%. These results, combined with the detection limit of 1.2 cfu/mL for the three target bacteria (confirmed by 134 mock samples), highlight the reliability of the instant biphasic assay, which avoids the need for any blood culture.

Next, we compare the pathogen identification time required by the biphasic assay with the identification time required in the clinical lab. Our biphasic assay has shown an average amplification time 42.5 minutes (FIG. 50). Adding this amplification time to the sample preparation time (90 min), the total time required for the identification of the bacteria using the biphasic assay was obtained. This total identification time was compared with the time needed in the clinical laboratory (time to positive culture plus identification by PCR). The overall (FIG. 31C) and species-specific (FIG. 31D) time to result are shown to illustrate the advantage of the biphasic assay in terms of response time. On average, while the biphasic reaction required 2.2 hours to achieve pathogen identification, the clinical laboratory required 23.2 hours. The t-test demonstrated a clear statistically significant difference between the results of the biphasic assay and the clinical practices (p-value <0.0001, FIG. 31C). This same behavior is observed when analyzing only the E. coli detection results (FIG. 31D).

Rapid and accurate identification of pathogens causing blood stream infections (BSIs) has remained a significant diagnostic challenge in healthcare, especially in conditions such as Sepsis where pathogen concentrations in blood can be as low as 1 cfu/mL. Due to the lack of rapid tests, blood cultures have remained the gold standard in diagnosing BSIs even though they take up to 5 days to produce results. It has also been shown that correct initial choice of antibiotic therapy within 1-3 hours from initial symptom-based sepsis recognition can significantly reduce mortality. There are only a few diagnostic platforms that bypass the need for blood culture but most of these platforms suffer from low and variable sensitivities due to inefficiencies in required conventional nucleic acid purification prior to detection in these platforms. The instant approach provides an alternative to blood processing and blood-based diagnostics for BSIs where we rapidly dry the blood with pathogen DNA to generate a dried blood matrix and then create a physical micro- & nano-fluidic network inside this dried blood matrix. Through simulations and experiments we show that this generated micro- & nano-fluidic network directly allows the amplification enzymes and primers to diffuse into the dried blood matrix, access the pathogen DNA and initiate amplification inside the dried blood matrix precluding any need for conventional nucleic acid purification. Further studies should be performed on understanding the mechanisms and confirming that the blood-drying protocol inactivates the inhibitors and keeps them in the solid phase, allowing the target nucleic acid amplification in the liquid phase. For example, measurement of heme or hemoglobin in the solid and liquid phase using ELISA or mass spectroscopy as a function of time and temperature could shed light into this hypothesis. This gives an extraordinary signal-to-noise and fluorescence change in our reactions which is comparable to purified DNA (no blood) reactions, even with more than 20% blood per reaction volume. This “biphasic” approach significantly lowers the time of analysis and reduces the associated instrumentation complexity and consumable costs (Table 2). We demonstrate our platform and the biphasic reaction approach on MRSA and E. coli cell-free DNA in whole blood and show single molecule reaction sensitivity (1copy/4 μL of blood dried per reaction). For cell-free DNA, we also show that compared to our biphasic reaction protocol, mixed blood reactions and reactions from only plasma post blood fractionation can have as much as 3 orders of magnitude higher (or worse) limit of detection. It is important to note that the optimized biphasic approach with small volumes of whole blood and moderate LODs is important in itself, for example, for finger pricks or heel lance nucleic acid testing in newborn blood samples⁵⁶. To detect a broad spectrum of pathogens and target the clinically relevant concentration range in sepsis, we coupled our biphasic, blood processing platform with mechanical bead lysis to disrupt the thick bacterial and Candida cell walls and allow access to DNA. In this format, we processed 0.8 ml of blood and showed a detection limit of 1.2 cfu/mL of blood for MRSA, MSSA, E. coli and Candida Albican. For E. coli, this detection limit is an order of magnitude improvement over the current state of the art E. coli detection limit of 11 cfu/mL in the only available FDA approved blood culture free diagnostic platform²². Our platform's superior detection limit can have a major impact especially for neonatal patients where studies have shown that concentrations in 68% of culture-positive cases fall below 10 cfu/mL^{41,42}. In contrast, since our method does not include purification and isolation of bacterial DNA since we directly dry the blood/blood lysate, we are able to capture and retain the few bacterial pathogens within the blood matrix. This results in a higher sensitivity in our biphasic method allowing detection of pathogens at low concentrations relevant to sepsis without culture⁵²⁻⁵⁵. We validated the biphasic assay by testing 63 clinical whole blood samples. As a result of this validation, our assay showed 100% agreement with clinical laboratory results in terms of sensitivity and specificity (no false positives were reported). Importantly, the average identification time using the biphasic approach (2.2 hours) was significantly shorter than the mean pathogen identification time in the clinical laboratory (23.2 hours).

Moreover, the current platforms require instrumentation such magnetic resonance reader and magnetic nanoparticles for detection of target post DNA amplification due to the high background from blood lysate and heme. In contrast, our platform only requires a centrifuge, a heater, a vortex, and a fluorescence reader for performing all the steps including blood drying, the micro- & nano-fluidic network generation, and isothermal biphasic amplification reaction using robust and commercially available Bst polymerase. With the minimum expertise such as accurate pipetting and possible contamination avoidance, these instruments have the potential to be optimized in automated manner to handle large volumes of the samples (˜5 mL).

We also demonstrate capability to detect genetic markers for drug resistance in pathogens by detecting the mecA gene in MRSA, which is responsible for its methicillin drug resistance. The current sample to result time in our platform is 2.5 hours with potential to go down to less than 2 hours with some automation. Also, assay time can be further reduced by skipping the thermal lysis step since drying-only protocol has shown the ˜1 cfu/mL of sensitivity. Our platform can easily be scaled to process 5 ml or more of blood to further improve the detection limit. Importantly, this platform can also be used to detect viral pathogens from whole blood where the option to culture the pathogen does not exist and rapid detection of 1 pfu/mL or lower are required. While we used vials for drying the blood and performing the reactions, cartridges akin to a ‘pixelated petri-dish’ with a larger area and shorter heights to accommodate 5 mL can provide for a more efficient drying of the blood. The clear supernatant in our reaction can allow visual or cell-phone camera based read out of pathogen amplification in our platform. Finally, we believe our platform will easily integrate into the current clinical workflow and significantly reduce costs and time to diagnosis of blood stream infections while providing state of the art sensitivity.

Materials and Methods:

DNA and Bacteria: Genomic DNA of Methicillin Resistant Staphylococcus Aureus (MRSA), strain HFH-30106, NR-10320, was obtained through BEI Resources. Genomic DNA of Escherichia Coli (O157:H7), NR-4629, was obtained through BEI Resources. These genomic DNA vials were aliquoted and stored at −80° C. Appropriate stock volumes were used either for direct experimentation or diluted to the right concentration in buffer or whole blood. For experiments using pathogenic bacteria, MRSA, strain HFH-30106, NR-10192, MSSA, strain MN8, HM-162, and E. coli (O157:H7), NR-4356 was obtained through BEI Resources. For experiments using pathogenic fungus, Candida Albicans, strain L26, NR-29445 was also obtained through BEI Resources. These bacterial and fungal glycerol stocks were stored at −80° C. Culture protocols in the next section.

Bacterial Culture: Media and agar plates were obtained from the Cell Media Facility at the University of Illinois Urbana-Champaign. Tryptic Soy broth and agar was used for MRSA culture and Luria-Bertani broth and agar was used for E. coli culture. Bacteria were grown in their respective broth at 37° C. for 16 hours overnight after which PBS stocks were prepared. C. albicans pathogens were grown in Yeast Peptone Dextrose broth at 30° C. for 16 hours overnight after which PBS stocks were prepared.

PBS stocks of pathogens were prepared in accordance with the work of Liao and Shollenberger⁶⁵. Briefly for bacteria, 250 μL of the overnight culture was centrifuged at 5000 g for 10 min to create a bacterial pellet, after which the pellet was washed twice with 1×PBS. Finally, the bacterial pellet was diluted in 1 mL of PBS, which was aliquoted and kept in room temperature. Each PBS stock was not used for more than 4 days post culture. PBS dilutions were done of the stock to the correct concentration and plated to know the bacterial concentration in the stocks. Based on the counts, the correct dilutions of the bacterial stocks were made in 1×PBS buffer or blood for the experiments. For fungus, PBS stock was prepared as described above and the 1 mL aliquot was kept in 4° C.; each stock was not used for more than 48 hours post culture. PBS dilutions were done of the stock and a hemocytometer was used to calculate the correct concentration of the pathogen, based on which the correct dilutions of the fungal stocks were made in 1×PBS buffer or blood for the experiments.

Blood Preparation and Drying

Whole venous blood samples are drawn from healthy, consenting, MRSA and E. coli negative adult volunteers with a syringe, which were later transferred to 6 mL BD Vacutainer K2 EDTA collection tubes. The tubes were stored in a sample rotisserie at 4C before using them for experiments.

Ten-fold serial blood dilutions of DNA or bacterial stocks were done to achieve the correct concentration required for experimentation. The spiked blood was then distributed into 0.2 mL PCR Reaction tubes (4 μL in each tube). This blood was dried on a hot plate at 37° C. for 20 min.

For blood drying characterizations, we tested different drying conditions, as summarized in Table 1. Samples of the dried blood pre and post thermal lysis are prepared, and SEM analysis is performed to quantify the porosity of the blood matrices. The details of the blood drying temperature, time, and corresponding porosity pre and post thermal lysis are summarized in Table 1.

Primer sequences: All primer sequences for the LAMP reactions were synthesized by Integrated DNA Technology (IDT). Primer sequences for MRSA mec A gene were obtained from Xu et al. and sequences for E. coli mal B gene were obtained from Hill et al^{48,49}. Primer sequences for C. albicans ITS2 region were obtained from Kasahara et al⁶⁴.

LAMP Reactions: LAMP assay was designed to target the mecA gene for MRSA and malB gene for E. coli, and ITS2 region for C. albicans. The LAMP assay is comprised of the following components: 1× final concentration of the isothermal amplification buffer (New England Biolabs), 1.025 mmol L⁻¹ each of deoxy-ribonucleoside triphosphates (dNTPs), 4 mmol L⁻¹ of MgSO₄ (New England Biolabs), and 0.29 mol L⁻¹ of Betaine (Sigma-Aldrich). These individual components stored according to manufactural instructions and a mix including all components was created fresh prior to each reaction. In addition to the buffer components, 0.15 μM of F3 and B3, 1.17 μM FIP and BIP, and 0.59 μM of LoopF and LoopB primers, 0.47 U μL⁻¹ Bst 2.0 WarmStart DNA Polymerase (New England Biolabs), 1 mg/ml BSA (New England Biolabs), and 0.74× EvaGreen (Biotium), a double-stranded DNA (dsDNA) intercalating dye, was included in the reaction. The final reaction volume was 16 μL. In case of buffer reactions, 1 μL of template in water or bacteria in 1×PBS buffer was added to make the final reaction volume 16 μL.

The format of biphasic blood reactions is as follow: In tubes with 4 μL of dried blood, 4 μL total of the buffer mix, BSA, and dye in the correct concentration are added so that final reaction concentrations are as mentioned above. Post thermal lysis, 12 μL total of buffer mix, BSA, dye, as well as primers and polymerase are added to make a final 16 μL reaction.

The format of mixed blood reactions with DNA spiked whole blood is as follows: In tubes with 4 μL of spiked blood, 16 μL total of the LAMP reaction reagents including primers and polymerase in the final concentrations mentioned above are added and mixed. For mixed reactions with supernatant of fractionated blood, 100 μL of DNA spiked blood was centrifuged at 5000 g for 10 min and supernatant with DNA was extracted and distributed in 4 μL aliquots into tubes. Thereafter, the 16 μL of LAMP reaction reagents were added in the final concentrations mentioned above and mixed.

All the LAMP tests were carried out in 0.2 mL PCR reaction tubes in an Eppendorf Mastercycler® realplex Real-Time PCR System. The tubes were incubated at 65° C. for 60 min in the thermocycler, and fluorescence data was recorded every 1 min. during the reaction. Eight replicates were done for each reaction.

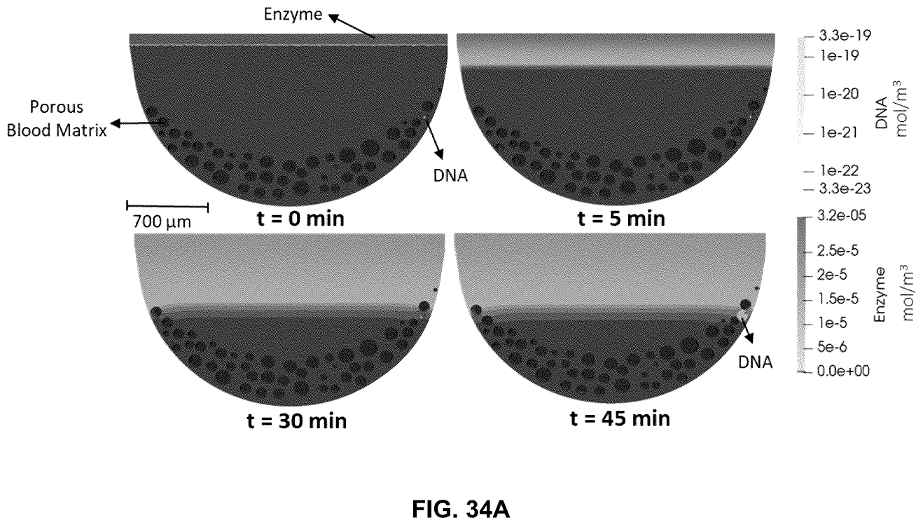

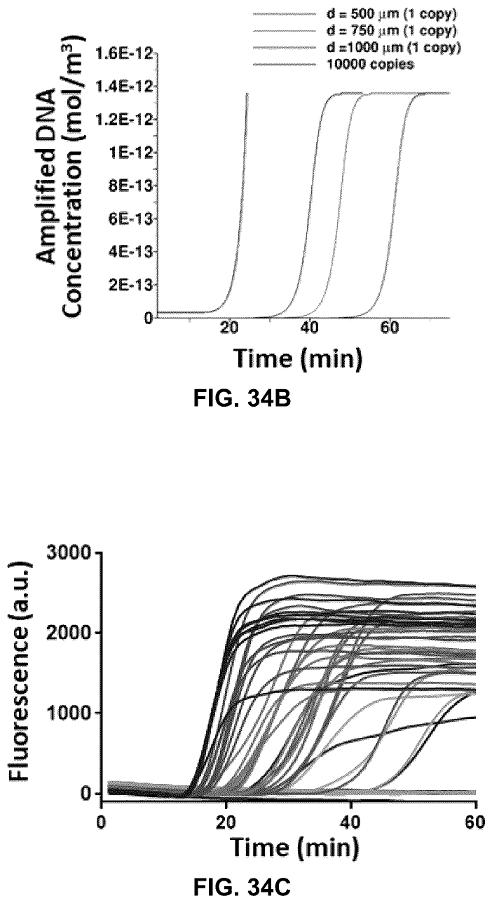

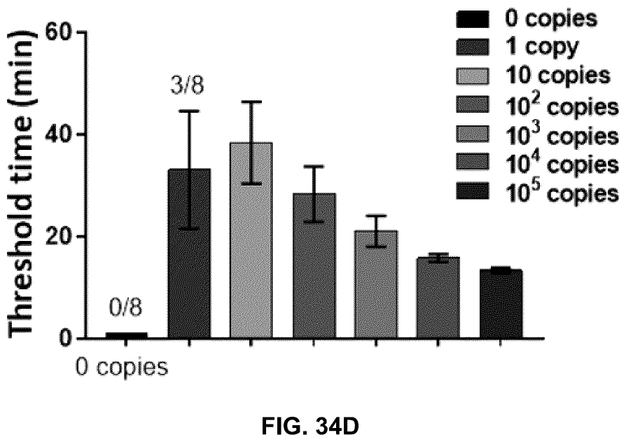

Reaction in blood cake: Reactions discussed in FIGS. 34A-34D conceptually showed that amplification starts within the porous channels of the blood cake. Experimentally, this was designed in the following format: In tubes with 4 μL of dried blood, 8 μL total of the buffer mix, BSA, and dye are added at the same final reaction concentrations mentioned above. Post thermal lysis, 4 μL of the supernatant was removed from the tubes. Then, 12 μL total of buffer mix, BSA, dye, as well as primers and polymerase are added to make the final reaction volume 16 μL.

High volume blood-processing with biphasic reactions: For high blood volume reactions and effective pathogen lysis, a bead beating protocol was used similar to that of T2 Biosystems⁶⁶. First, in a 1.5 mL tube, 800 μL (or 1 mL) of blood with the correct concentration of pathogens was added to a tube with 40 μL (90 mg) of glass disruptor beads (Scientific Industries, Inc.) and 600 μL of blood lysis buffer (consisting of 10 mmol L⁻¹ KHCO₃, 150 mmol L⁻¹ NH₄Cl, and 0.1 mmol L⁻¹ EDTA).The blood and lysis buffer were manually pipetted and left to incubate at room temperature for 5 min. After centrifugation at 6000 g for 10 min, the lysed blood supernatant was removed and 200 μL of TE buffer was added to the tube for bead lysis. The tubes were vortexed at 3000 rpm for 10 min. Finally, after a brief 10 second centrifugation, the 30 μL of lysate was distributed from the 1.5 mL tubes into as many 0.2 mL PCR-Reaction tubes as necessary to extract the complete lysate. This aliquoted lysate was dried at 95° C. in a heater for 10 min and the biphasic reaction protocol was followed thereafter. For these reactions, the format was as follows: In tubes with 30 μL of dried lysate, 72 μL of the buffer mix, BSA, and dye in the correct concentration were added so that final reaction concentrations were as previously mentioned. Post thermal lysis, 24 μL total of buffer mix, BSA, dye, as well as primers and polymerase were added to make a final 96 μL reactions. These assays were incubated at 65° C. for 60 min in the thermocycler, and fluorescence data was recorded every 1 min. during the reaction.

For mixed blood reactions, in which blood lysate post blood processing and bead beating was not dried, the reaction format was as follows: In tubes with 30 μL of lysate, 66 μL of the LAMP reaction mix components including primers and polymerase were added in the correct concentration so that the final 96 μL reaction concentrations were equivalent to that previously mentioned. The maximum recommended liquid capacity for PCR tubes is 100 μL. The assays were incubated at 65° C. for 60 min in the thermocycler and data was collected.

For effective lysis of C. Albicans fungal pathogens, the blood lysis and bead beating protocol was followed as mentioned above, but with 500 μm glass disruptor beads (Scientific Industries, Inc.). For the biphasic reaction done with dried blood lysate, reaction composition was the same as that previously mentioned for 96 μL reaction and incubation for the amplification occurred at 62° C. for 60 min, which was later optimized to occur at 67° C. for increased specificity of the assay. These reactions were further optimized by decreasing the final concentration of each primer in the 96 μL reaction: 0.04 μM of F3 and B3, 0.33 μM FIP and BIP, and 0.17 μM of LoopF and LoopB.

Amplification data analysis: The off-chip raw fluorescence curves and amplification threshold bar graphs were analyzed using a MATLAB script and plotted using GraphPad Prism. The threshold time for each curve was taken as the time required for each curve to reach 10% of the total intensity. The amplification threshold bar graphs are show a mean of 8 samples.

Clinical samples: The clinical samples were discarded whole blood samples from patients in the ED that have a blood culture ordered. Samples were collected at Carle Foundation Hospital (Urbana, IL) through an approved institutional review board study (Carle IRB #21BIO3462). The samples were transferred to UIUC and stored at 4° C. until use. The clinical procedure for pathogen identification can be seen in the supplementary information (Methods 1).

Table 1. Summary of blood drying conditions. Thermal lysis in all conditions was performed at 95° C. for 2 minutes. Porosity values are presented as mean and standard deviation of 3 samples (n=3).

TABLE 1
Summary of blood drying conditions. Thermal lysis in all conditions was
performed at 95º C. for 2 minutes. Porosity values are presented as mean and standard
deviation of 3 samples (n = 3).

| | | | | | Porosity | Porosity | |
|---|---|---|---|---|---|---|---|
| | | Blood | | | Pre | Post | |
| Volume | Mechanical | Drying | Drying | Drying | Thermal | Thermal | Detection |
| Dried | Bead Lysis | Method | Temperature | Time | Lysis (%) | Lysis (%) | Limit |
| 4 μL- | No | Hot plate | 37° C. | 15-20 | 6.45 ± 1.18 | 68.60 ± 1.57 | 1 copy/4 μL |
| Whole | | with low | | min | | | whole |
| Blood | | vacuum | | | | | Blood |
| | | (5 mmHg) | | | | | |
| 4 μL- | No | Hot plate | 37° C. | 20 min | 16.34 ± 1.17 | 60.18 ± 0.41 | 1 copy/4 μL |
| Whole | | only | | | | | whole |
| Blood | | | | | | | Blood |
| 4 μL- | No | Hot plate | 50° C. | 10 min | 11.8 ± 1.46 | 61.69 ± 0.47 | 1 copy/4 μL |
| Whole | | only | | | | | whole |
| Blood | | | | | | | Blood |
| 4 μL- | No | Hot plate | 95° C. | <5 min | 6.59 ± 0.22 | 7.00 ± 0.53 | 1000 |
| Whole | | only | | | | | copies/4 μL |
| Blood | | | | | | | whole |
| | | | | | | | Blood |
| 30 μL- | Yes | Hot plate | 95° C. | 10 min | 11.55 ± 0.27 | 63.83 ± 1.02 | 1 |
| Blood | | only | | | | | cfu/800 μL |
| Lysate post | | | | | | | whole |
| bead | | | | | | | Blood |
| beating | | | | | | | |

TABLE 2
Consumable cost calculation for processing 1 patient sample (0.8-1
mL blood) in a biphasic reaction. The table includes all consumable
products and reagents required for the processing 0.8-1 mL blood
in the bead-beating based biphasic LAMP reaction.

| Biphasic | | Amount or | | Price of |
|---|---|---|---|---|
| Reaction | | Concentration | | Processing 1 |
| Process | | Required for | | patient sample |
| Flow Step | Materials/Reagents | each reaction | | (0.8-1 mL of blood) |
| All Steps | Pipette Tips | ½ Box (48 tips) | | $7.22 |
| | 1.5 mL Microcentrifuge Tubes | 15 | tubes | $2.17 |
| | 0.2 mL, Optical 8-Tube Strip | 1 | strip | $0.84 |
| RBC Lysis | Screw Cap Micro Tube (1.5 mL) | 1 | tube | $0.13 |
| and Bead | 0.1/0.5 mm Disruptor Beads | 90 | mg | <$0.01 |
| Beating | KHPO3 - ACK Lysis Buffer | 10 | mM | <$0.01 |
| | NH4CL - ACK Lysis Buffer | 150 | mM | <$0.01 |
| | EDTA - ACK Lysis Buffer | 0.1 | mM | $0.01 |
| | H2O - ACK Lysis Buffer | N/A | | $0.04 |
| | TE Buffer | 1X | | $0.01 |
| Biphasic | Primers | Range: 0.2-1.6 μM | | $1.41 |
| LAMP | Polymerase *** | 470 | U/mL | $14.18 |
| Reaction | Dye | 925 nM (0.74X) | | $1.36 |
| | BSA | 1 | mg/mL | $2.07 |
| | Betaine | 0.29M | | $0.81 |
| | dNTPs | 1.025 | mM | $6.46 |
| | H2O | N/A | | $0.09 |
| | *** Amplification Buffers are | Total | | $36.80 |
| | included | | | |

Results 1. Numerical Simulation and Experimental Validation of Biphasic Reaction Mechanism

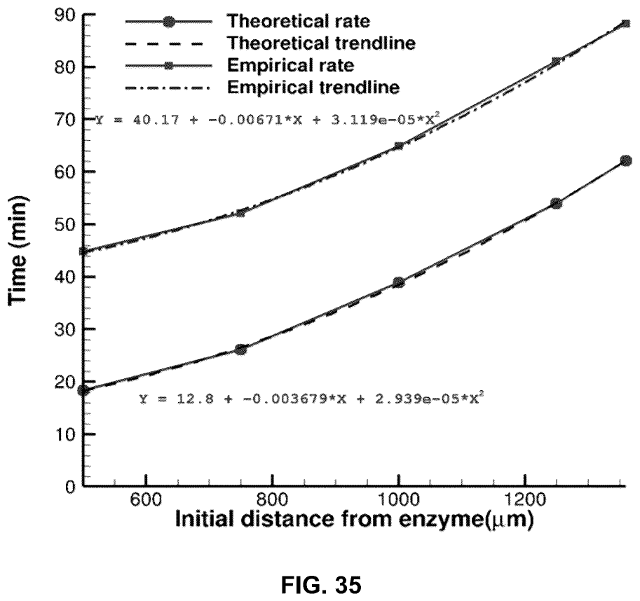

To further dissect the mechanism of biphasic amplification, we perform simulations to recreate the dried blood matrix and micro- & nano-fluidic networks captured in SEM images (porosity 66.8%) and simulate the diffusion of enzymes through these networks to access DNA and initiate amplification. In our simulations for a single DNA copy in dried blood matrix, the location of the pathogen DNA is varied, and the simulation is carried out for different distances between location of the DNA and the enzyme. In all the simulations, we observe that only the enzyme (Bst polymerase) and the raw materials (dNTP, primers) diffuse into the micro-nano channels, reach the DNA and start the replication reaction, whereas the pathogen DNA does not diffuse out⁴⁶. This is because the pathogen DNA has a diffusion coefficient (2.82e-14 vs. 5.63e-11 m/s) which is three orders of magnitude smaller than that of the other components of the system⁶⁷ (FIG. 34A). Initially, the enzyme starts at a specific distance from the single pathogen DNA molecule, which we simulated from 500 to 1000 μm. Note that the blood matrix itself is only 20-40 μm thick based on the SEM images but we simulated for much longer distances since the polymerase and primers are added post thermal lysis and mixing in the vicinity of blood structures might not be adequate. The concentration vs. time curves for 10,000 copies of DNA were also compared with 1 copy of DNA at different initial distances from the enzyme (FIG. 34B). For simulating 10,000 copies of DNA, we distribute the DNA uniformly across the blood matrix, whereas in the case where 1 copy of DNA is simulated, we place the DNA at specific locations in the blood matrix. We observe that for 10,000 copies, the DNA amplification takes around 20 minutes, whereas 1 copy of DNA starts amplification at around 30 minutes and can go up to 60 minutes, depending on the initial distance of the enzyme from the DNA. For the rate of amplification of DNA used in the final transport equation (Equation 1, methods) in the simulation, we use the empirical rate which is calculated from the experimentally purified DNA amplification data by normalizing and fitting it to a sigmoidal curve of the form mentioned in Equation 3 in methods. The comparison of theoretical and empirical rates for simulated amplifications is shown in FIG. 35. Details for the equations and their parameters are provided below.

To experimentally confirm the biphasic mechanism, we discarded the supernatant after the thermal lysis and only amplified the DNA in the blood cake after adding the polymerase and primers (Methods Section). We observed robust amplification down to a single copy implying that for low copies, none of the DNA was extracted in the supernatant and the enzymes can initiate the amplification within the blood matrix. Also, the simulation results demonstrate that for low copy number of DNA, the amplification time depends on the initial distance between the enzyme and the DNA. This is also corroborated in experimental results by the larger range of amplification threshold times (10-20 minutes) for low DNA copy numbers (FIGS. 34C-34D). MRSA DNA was used in these experiments.

Simulation data: To understand the biphasic reactions, the system was modeled as a continuum, where the experimental process was divided into three main steps:
Thermal Lysis: Performed at 95° C.; this process was used to create the microchannels in the dried blood matrix.
Diffusion: Performed at 65° C.; this process consisted of adding the enzymes (BST polymerase) and the raw materials (dNTP) to the porous dried blood matrix and allowing them to diffuse into the microchannels.
Amplification: This process comprises the reaction taking place between the pathogen DNA and dNTP in the presence of BST polymerase, replicating a segment in the DNA sequence of the pathogen.

The dimensions of the experimental setup were of the order of millimeters; therefore, we used continuum simulations to model the system. We first created the geometry, which was modeled after the standard 0.2 mL PCR tube with 4 ul of blood dried in it for the biphasic reaction. To model the micro-nano fluidic networks within the blood matrix, we performed image segmentation in MATLAB of SEM images of the dried blood in PCR tubes post thermal lysis to calculate porosity of the sample. A constant threshold was used for image segmentation in MATLAB for pre and post thermal lysis SEM to characterize the difference in porosity. The flow and the dynamics of the system were simulated using the continuum transport equation given below (Equation 1). These simulations have been performed in OpenFoam⁶⁸ with a mesh size of 0.001 mm. Backward Euler method is used to evaluate the time derivatives, and Gaussian linear and linear-upwind schemes are used for evaluating the divergence and Laplacian, respectively. The time-dependent convection-diffusion-reaction equation is given by,

dcᵢ/dt−∇·(U·cᵢ)−∇·(Dᵢ·∇cᵢ)=Rᵢ (1)

where cᵢ is the concentration of species i, U is the velocity of the fluid in consideration, Dᵢ is the diffusion coefficient of species i and rate of the reaction, Rᵢ is initially given by

$${R}_{i} = \frac{ln 2}{τ} \times {c}_{i} \times {2}^{τ} \qquad ( 2 )$$

where τ is the time constant of the reaction.

The system was maintained at 1 atm pressure. The velocity of the fluid (U) is zero in our system as there are no pressure gradients and the electro-osmotic velocity is negligible. The temperature of the system was at 95° C. for the first 2 minutes of the simulation, where only the pathogen DNA could diffuse to model the thermal lysis process of the experiment. After 2 minutes, the temperature is maintained at 65° C. for 60 minutes to mirror the experimental conditions. The system is modeled using 2 species, namely the BST polymerase enzyme and the pathogen DNA. These species are assumed to be in aqueous medium, though the actual experimental setup consists of a buffer solution. In addition, we assume that the charged species in the solution do not affect the diffusion of the enzyme and dNTP into the blood matrix. This is because the Debye length, which is a measure of how far a charged species' electrostatic effect persists, calculated for this system is close to 5 nm, whereas the pores in the blood matrix formed post-lysis are of the order of micrometers (μm), as can be seen from the SEM images in FIG. 27. So, the electrostatic effects and the electro-osmotic velocity are negligible.

To be able to predict the time required for amplification in a porous blood matrix, we used experimental data to obtain an empirical rate equation, which could be used to predict how the reaction proceeds in similar media. FIG. 35 shows a comparison between the times of amplification for theoretical and empirical rates used in the transport equation. The x-axis is the initial distance between the enzyme and the pathogen DNA. For modeling the rate of the equation used in Eq. 1, we use the empirical rate equation which is calculated from the experimental data by normalizing the data and fitting it to a sigmoidal curve of the form given below. The empirical rate equation was compared with the theoretical rate given by Equations 2 and 3 and was used to predict the times of amplification for similar reactions in different geometries.

It can be seen that though the amplification times obtained with the empirical rate are different than that when compared to the theoretical rate, the trends for both the cases match very well, i.e., we are able to capture the physics of the theoretical rate with our empirical rate. Further, the way the theoretical rate is calculated does not represent our biphasic dried blood system very well, so the times of amplification are different.

$${R}_{i} = 0.0078 \times [ DNA ] \times ( \frac{1}{1 + {2}^{- \frac{( t - 1269 )}{166.32}}} ) \qquad ( 3 )$$

Results 2. Characterization of Biphasic Reaction Using Cell Free DNA

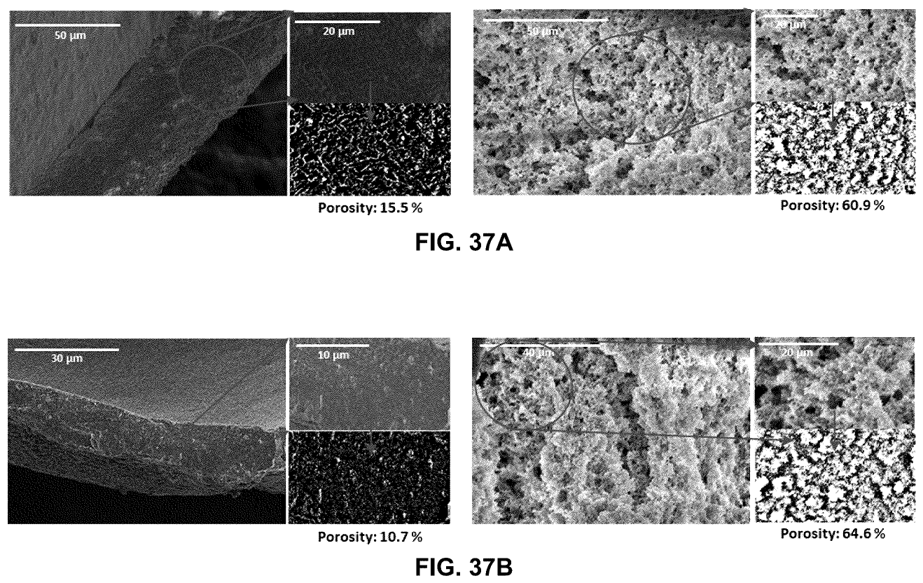

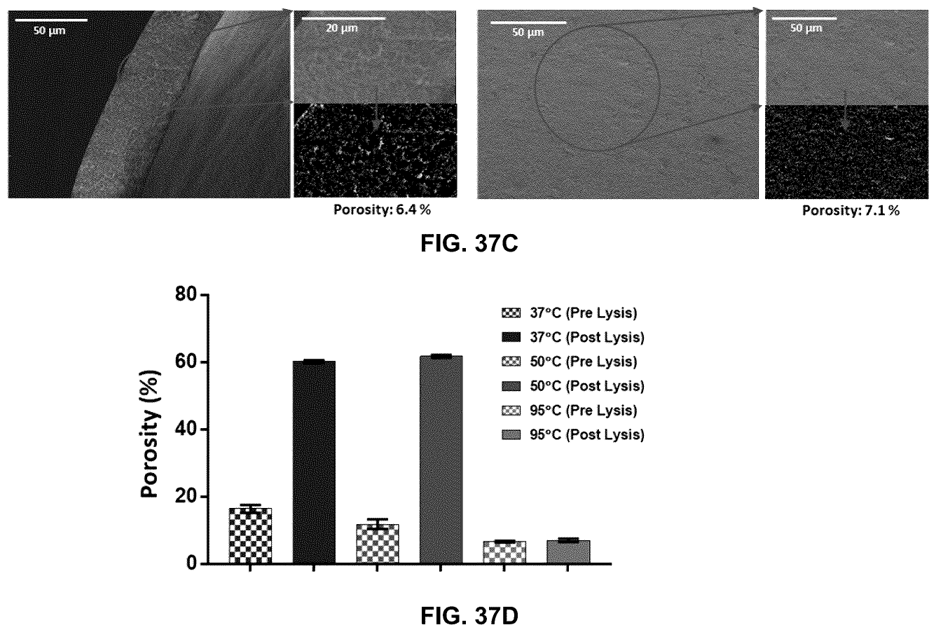

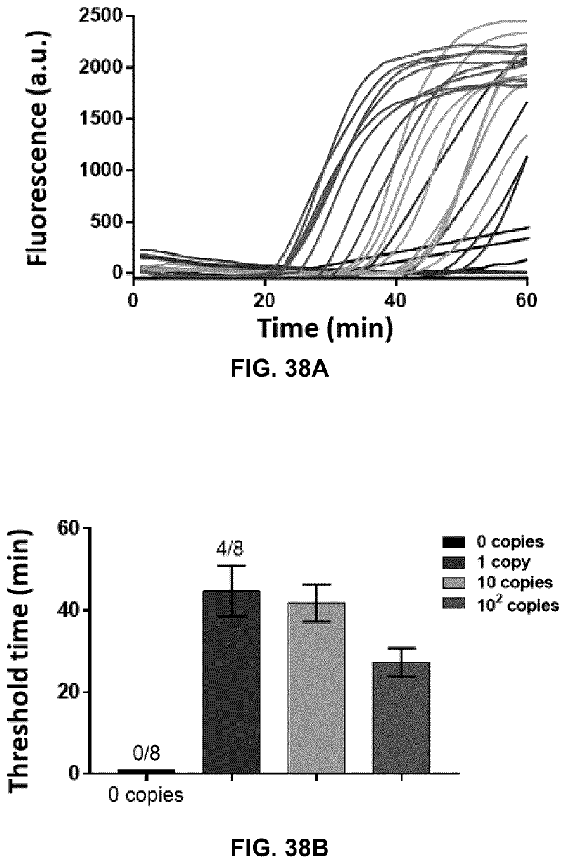

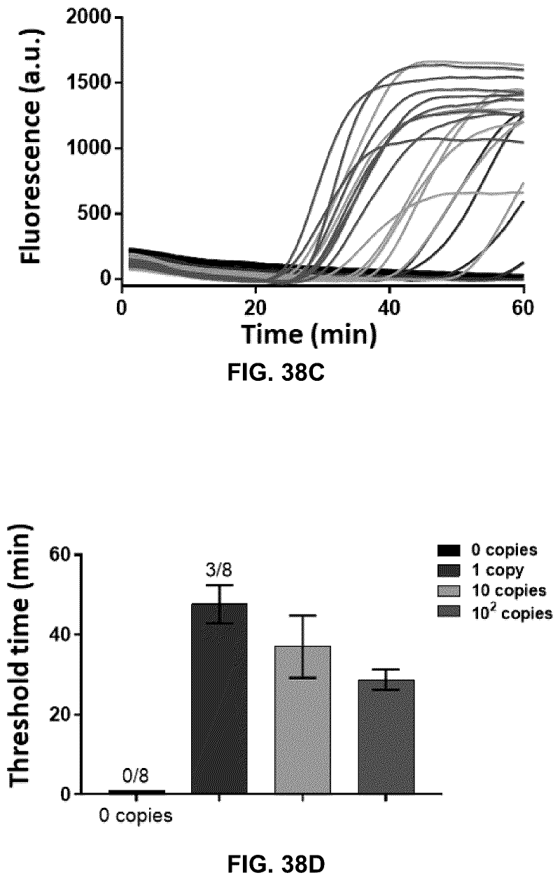

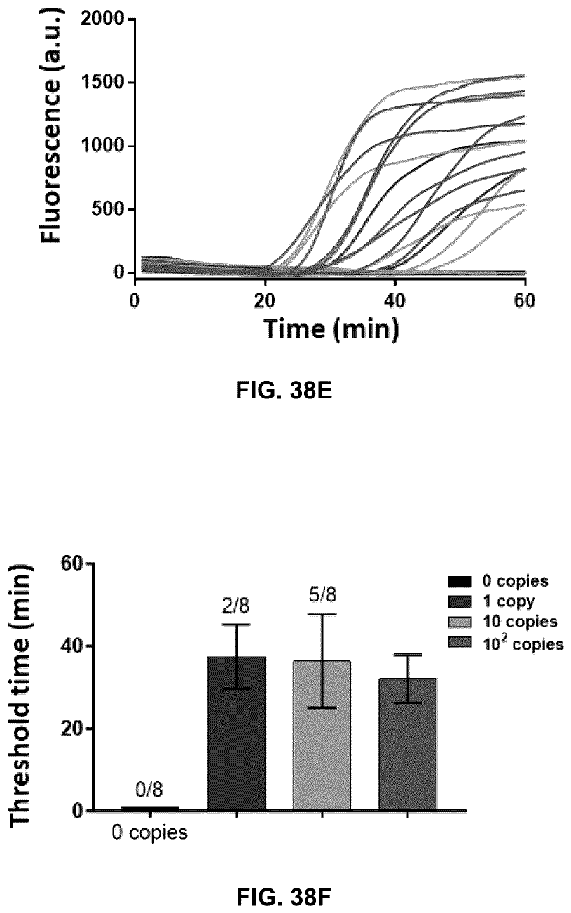

We tried different drying conditions and a complete list of drying parameters and its impact on porosity of the dried matrix and the detection limit of the biphasic assay is shown in the Table 1. The SEM analysis of porosity is found at FIGS. 37A-37D. As can be seen from the analysis, for whole blood, drying temperatures of 37° C. and 50° C. gave similar results in terms of porosity and associated detection limits in DNA amplification (˜1 copy/4 μl). However, when whole blood was dried at 95° C., we saw that the porosity of the dried matrix pre- and post-thermal lysis did not change appreciably (6.6% vs 7%), while resulting in ˜2 orders reduced detection limit of 100 MRSA DNA copies in 4 μL of dried whole blood (FIGS. 38A-38F). This is likely because of increased crosslinking of the blood matrix at high drying temperatures.⁵⁰.

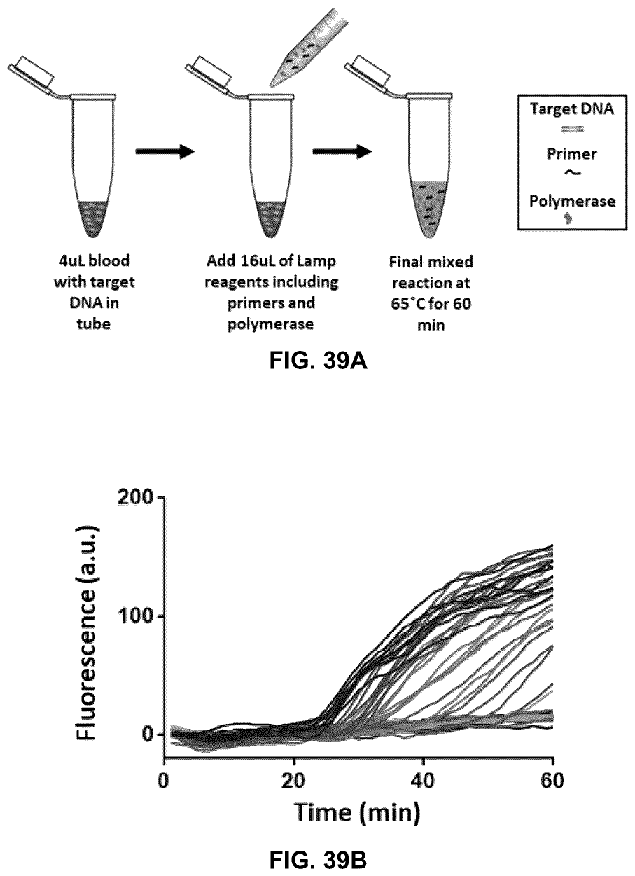

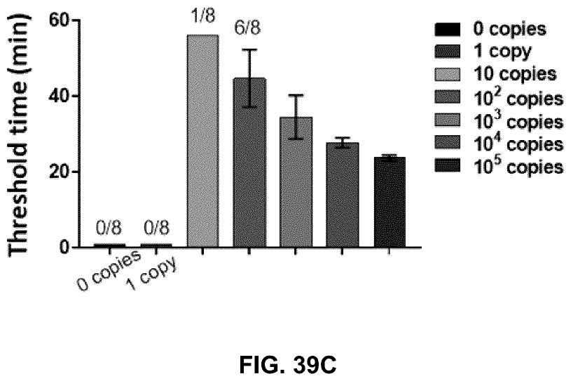

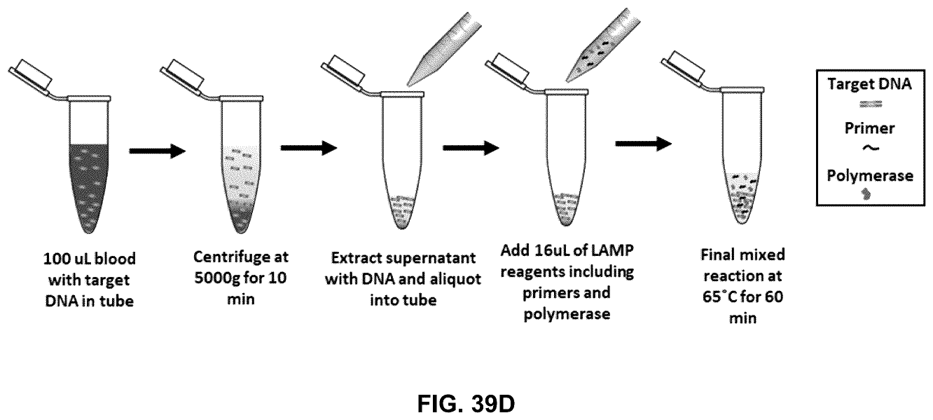

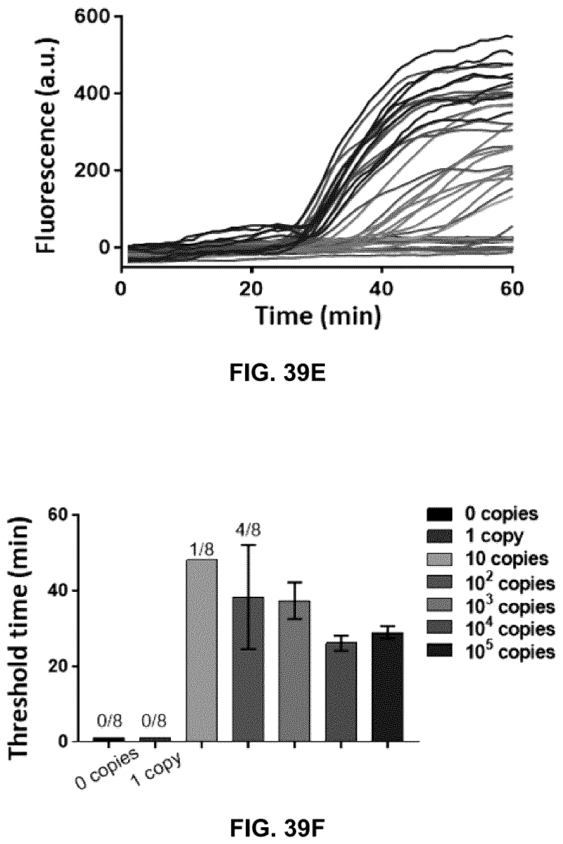

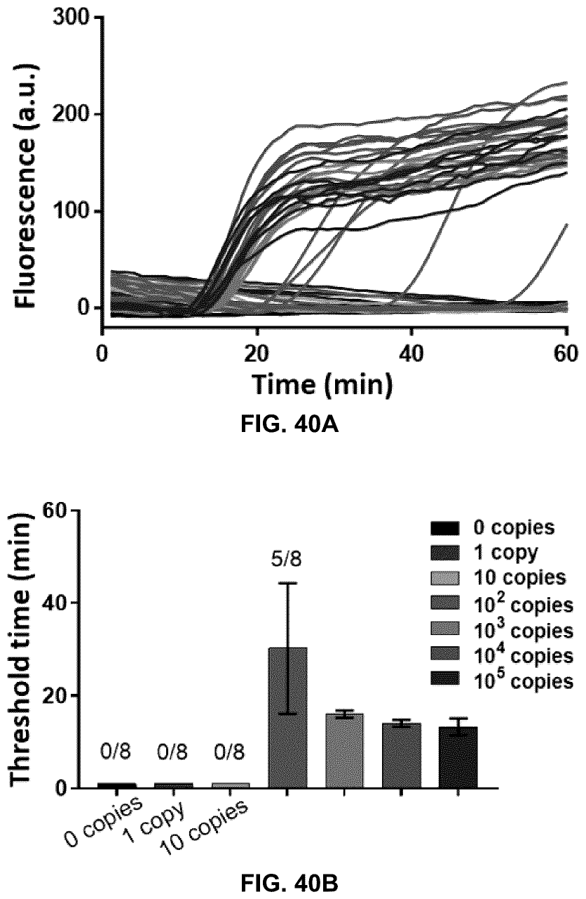

Next, we compared the sensitivity of our biphasic assay with other standard blood processing protocols. The protocol and amplification results in mixed whole blood LAMP reactions are shown (FIGS. 39A-39C and FIGS. 40A-40B). The reactions were performed with 4 μL of blood with MRSA DNA spiked at different concentrations and 16 μL of LAMP reaction mix to allow for a direct sensitivity comparison with our biphasic reactions. The amplification fluorescence curves, and the threshold times demonstrate a detection limit of 1000 copies per 4 μL of blood (only 6/8 repeats amplify in 100 copies/4 μL) (FIGS. 39B-39C). This is essentially 3 orders of magnitude worse than the limit of detection in our biphasic reaction format with identical blood and reaction mix volumes. This result highlights the inhibition of a LAMP reaction due to the proteins and cellular components in whole blood when they are mixed and dispersed in the final reaction. This demonstrates the importance of inactivation of these inhibitory elements when they are dried in our biphasic format.

To evaluate if cell free DNA in blood can simply be extracted from the supernatant plasma after centrifugation and fractionation of blood, we spiked MRSA DNA at different concentrations in whole blood at identical concentrations to biphasic reaction format to allow direct comparison. However, in this experiment, we fractionated the blood by centrifuging at 5000 g for 10 minutes and extracted 4 μl of the supernatant plasma to then be used with 16 μL of LAMP reaction mix, to allow for a direct sensitivity comparison with our biphasic reactions (FIG. 39D). The amplification curves and the threshold times show a limit of detection of 1000 copies/4 μl of supernatant (FIGS. 39E-39F). This, again, is 3 orders of magnitude worse than the limit of detection in our biphasic reaction format highlighting the important fact that for low copies of cell free DNA in blood, not all of the DNA is recovered in the plasma.

Results 3. Control Experiments and Analysis of Heme Content for Large Volume Reaction

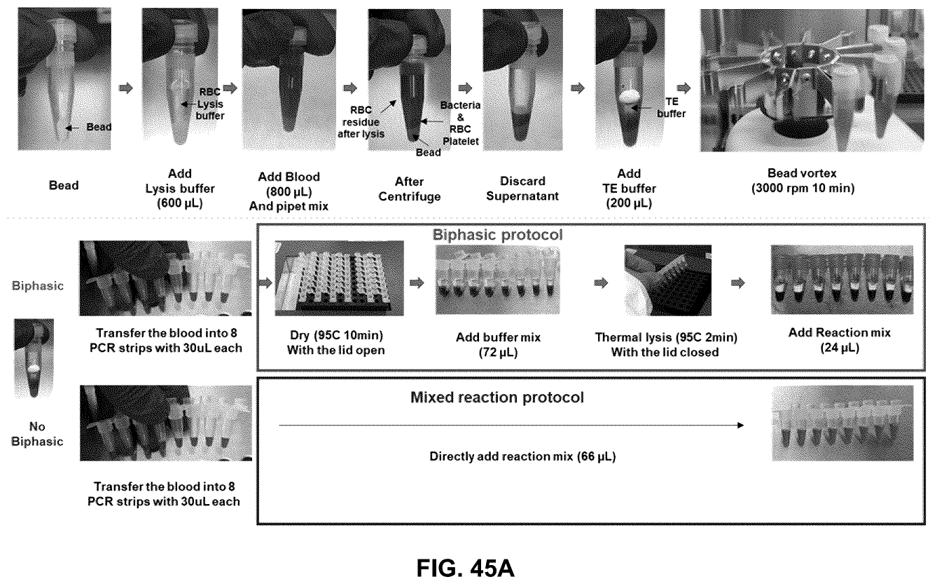

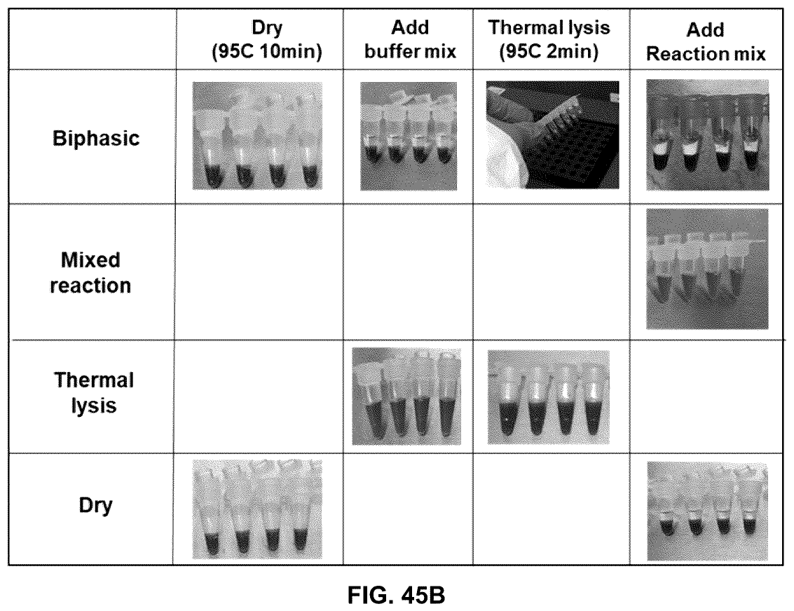

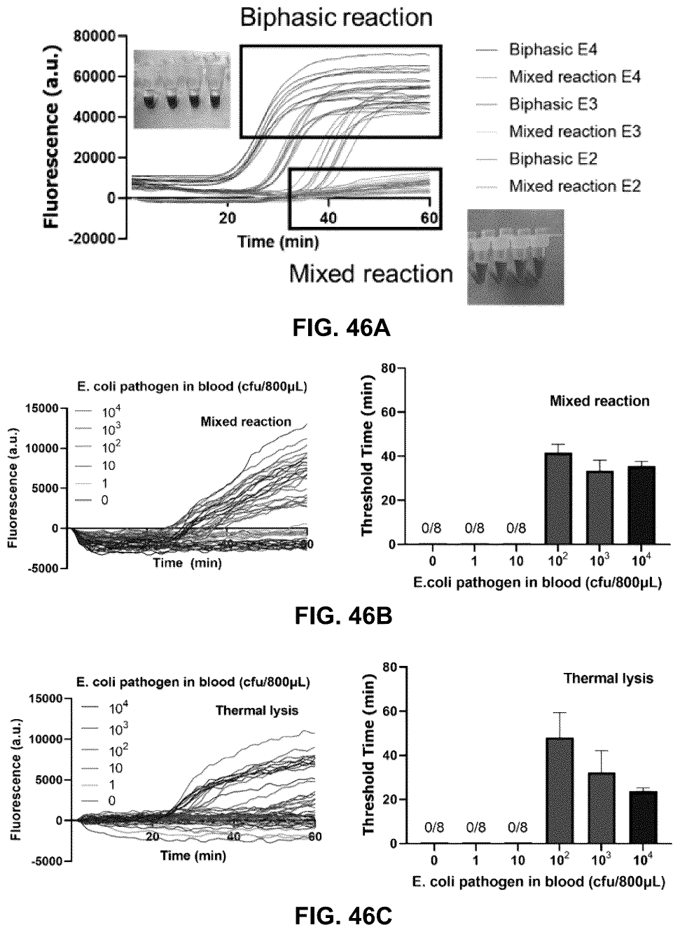

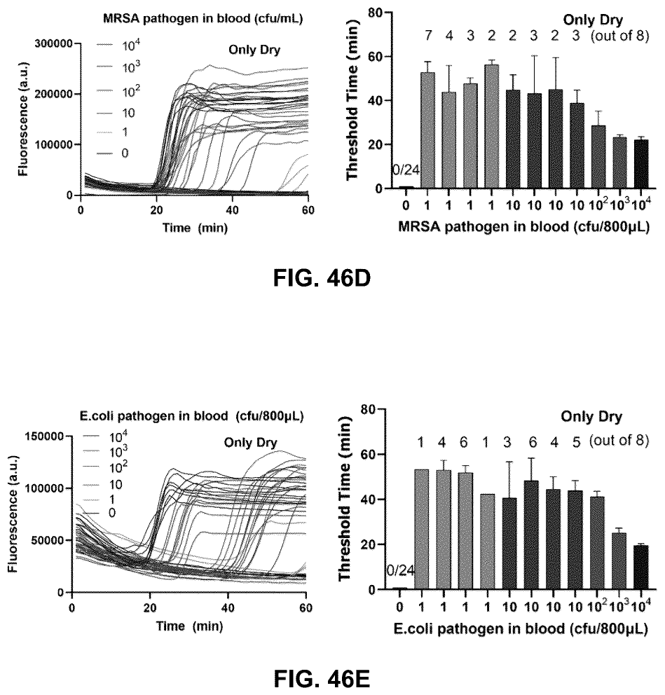

We compare the sensitivity of our biphasic assay with other standard blood processing protocols. Comparison of control protocols after each experimental step is shown with optical images (FIGS. 45A-45B). This shows an actual representation of all protocol steps that were used for measurement of control experiments, mock samples, and patient samples (FIG. 45A, FIG. 27, and FIG. 30A). After blood lysate post mechanical lysis is transferred (30 μL) to PCR tubes, the biphasic protocol consists of two major steps: drying and thermal lysis. To determine the effect of each process on LOD, control experiments were performed in which one (dry or thermal lysis) or both processes were omitted. Comparatively, a mixed reaction (no dry and no thermal lysis) performed with blood lysate post bead-based mechanical pathogen lysis of E. coli spiked in 800 μL whole blood (blood not dried) in the same LAMP reaction format, showed 10 times lower (˜6,000) overall fluorescence change than biphasic reaction (˜60,000) after the amplification (FIG. 46A). Also, the mixed reaction showed a detection limit of >1.2e2 cfu/mL (FIG. 46B). When tested with the thermal lysis step only (no drying), the reaction showed a same detection limit as the mixed reaction (FIG. 46C), suggesting that thermal lysis had a limited effect on the extraction of DNA from the bacteria. Interestingly, drying-only (no thermal lysis) reaction with blood lysate post bead-based mechanical pathogen lysis of both E. coli and MRSA was able to reach the detection limit of 1.2 cfu/mL (FIGS. 46D-46E). This suggests the pretreatment of RBC lysis, bead-based mechanical pathogen lysis and following centrifugal removal of the supernatant might help to reduce inhibitor components in the blood, thus contributing to the increased sensitivity despite the absence of further thermal lysis step. These results also imply that the assay time can be reduced by skipping the second thermal lysis from the protocol and that the porosity of micro/nano structure in blood cake may have a minor effect on assay sensitivity if mechanical lysis is introduced prior to the biphasic protocol.

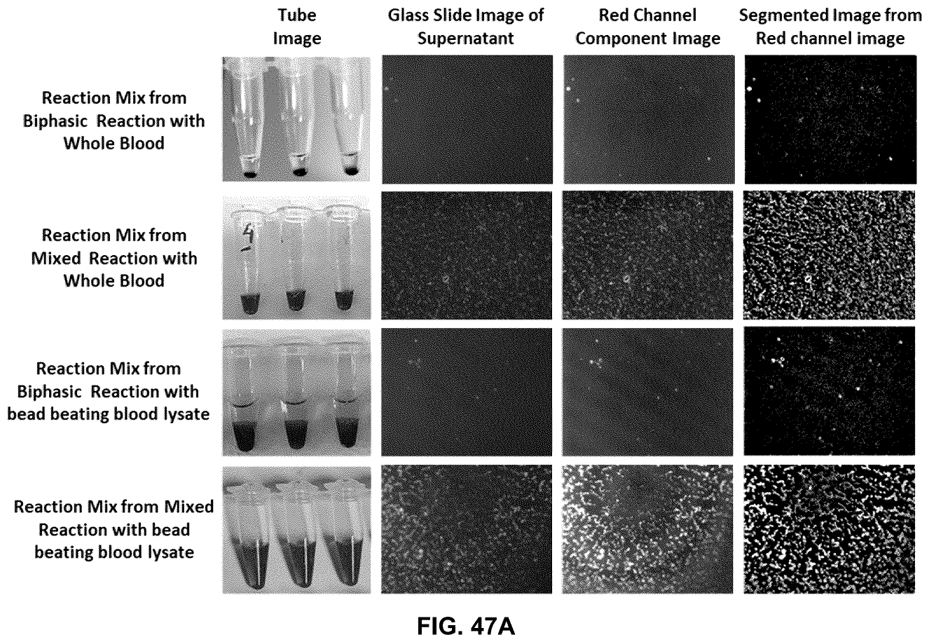

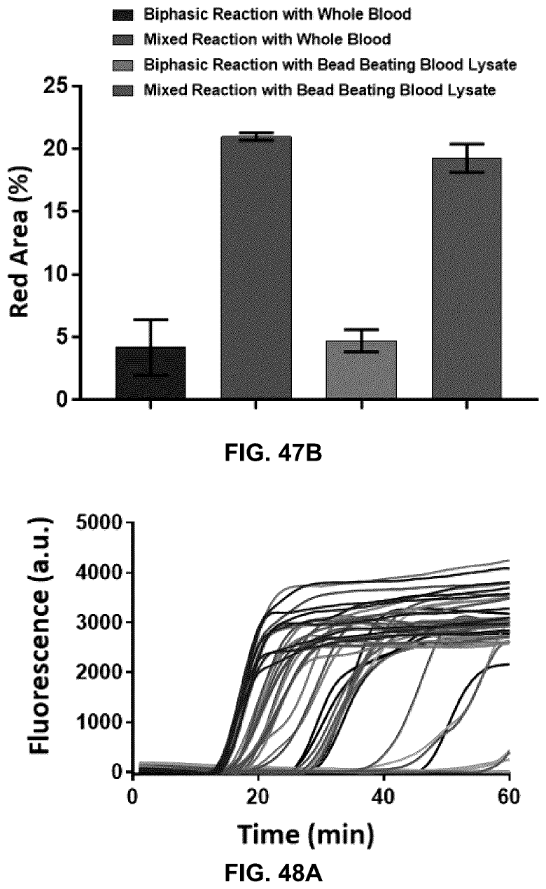

Moreover, to show that heme released due to RBC lysis in the bead beating protocol remains within the dried blood region and does not interfere with the reaction, we performed an analysis of the reaction supernatant in our 2 biphasic protocols (with and without bead beating). The analysis can be found in the supplementary information (FIGS. 47A-47B). Briefly, the reaction mix of the biphasic reactions with and without bead beating was extracted and analyzed quantitatively for “red content” on a glass slide as heme is responsible for the red color in blood^{59,60}. The percentage of heme content in both biphasic protocols was ˜5% as opposed to ˜20% for mixed (not biphasic) reaction formats. The reduced heme content in the supernatant allows for the high signal to noise ratio (high fluorescence change) seen in our reactions. Clear supernatant phase can be seen in the biphasic format explaining the high fluorescence change and also allowing the possibility of visual, instrument-free detection using HNB or Calcein dyes for resource starved settings in the future^{61,62} Moreover, we also demonstrate capability to detect genetic markers for drug resistance in pathogens by detecting the mecA gene in MRSA, which is responsible for its methicillin drug resistance. The time from sample to result in our assay is less than 2.5 hours (blood drying time=10 mins, RBC lysis and Bead lysis=30 mins, Reagent preparation for amplification and operational pipetting time=30 mins, amplification time=60 mins or less) with further possibility for reduction to less than 2 hours. For details on the equations and their parameters, see the supplementary notes below. For details on the methods, see the supplementary notes below.

SEM and Heme content analysis: For SEM characterizations, dried blood in PCR tubes and thermally lysed blood matrix were first fixed using 2.0% paraformaldehyde and 2.5% glutaraldehyde in PBS. Post fixation, the samples were rinsed with PBS and dehydrated with ethanol. The samples were finally prepared for critical drying in 100% ethanol and kept in 4° C. until the critical drying process. Post critical drying, the samples were sputter coated with gold-palladium using the Desk-II TSC instrument and studied using a FEI Quanta FEG 450 ESEM imaging instrument. SEM images were taken at different magnifications for each of the samples. Image segmentation of these SEM images were performed using a constant threshold to calculate the porosity of the sample in MATLAB.

To show that heme content released due to RBC lysis in the bead beating protocol still remains locked at the bottom dried phase and does not interfere with the reaction, we performed a quantitative heme content analysis of the reaction supernatant in our 2 biphasic protocols (with and without bead beating) as well as their corresponding mixed reaction protocols. 10 uL of the reaction mix for the biphasic and mixed reactions were extracted onto glass slides and imaged in brightfield using an inverted microscope at 40× magnification. The glass slide images were then processed through Image J for the red channel component, as heme is responsible for the red color in blood^{59′60}. In the red channel component image, the white areas indicate high concentrations of heme content, which was quantified through image segmentation in MATLAB.

Methods 1. Clinical Procedure for Pathogen Identification

After whole blood samples were drawn from patients, blood cultures were performed using the BACTEC™ FX Blood Culture System (Becton Dickinson). A set of two bottles (aerobic and anaerobic) were incubated and times to positive culture were recorded. Culture was performed until a positive alarm sounded from the machine. Culture was performed for 5 days in negative case.

Pathogen identification after positive cultures consisted of two categories. Both pathways included brief Gram-staining tests (˜15 mins). It was performed using the automated Gram stainer (PREVI® COLOR GRAM, bioMerieux) to avoid subjective readout. In the first way, multiplexed PCR (2 hours and 45 mins) was performed using the BioFire® FilmArray® Torch System (bioMerieux). Results included data for types of species and whether they are detected or not detected. Total time to result was 3 hours. For the second pathway, sub-culture was conducted with agar plates to see actual bacterial growth. Overnight culture was conducted with a few drops (up to 100 μL) of culture-positive blood. Afterwards, pathogen identification and antibiotic susceptibility tests were performed during overnight using the VITEK® 2 (bioMerieux). Total time to result for second pathway is 3 days.

Combined results from two pathways were reported to the system. For the comparison of the assay performance, the time to result from the first pathway (3 hours) was added to the time to culture positive (different from sample to sample) and then compared with the time to result from biphasic approach. From blood culture to pathogen identification, all steps were conducted by trained personnel of Carle foundation hospital.

### References Associated with Example 5

See also, A. Ganguli et al. “A culture-free biphasic approach for sensitive and rapid detection of pathogens in dried whole-blood matrix.” PNAS 119(40): e2209607119 (Sep. 26, 2022) (and supporting information/materials), which is specifically incorporated by reference herein for Example 5.
1. Sinha M, Jupe J, Mack H, Coleman T P, Lawrence S M, Fraley S I. Emerging technologies for molecular diagnosis of sepsis. Clin Microbiol Rev. 2018; 31(2). doi:10.1128/CMR.00089-17
2. Peker N, Couto N, Sinha B, Rossen J W. Diagnosis of bloodstream infections from positive blood cultures and directly from blood samples: recent developments in molecular approaches. Clin Microbiol Infect. 2018; 24(9):944-955. doi:10.1016/j.cmi.2018.05.007
3. Singer M, Deutschman C S, Seymour C W, et al. The Third International Consensus Definitions for Sepsis and Septic Shock (Sepsis-3) HHS Public Access. JAMA. 2016; 315(8):801-810. doi:10.1001/jama.2016.0287
4. Novosad S A, Sapiano M R P, Grigg C, et al. Vital signs: Epidemiology of sepsis: Prevalence of health care factors and opportunities for prevention. Morb Mortal Wkly Rep. 2016; 65(33):864-869. doi:10.15585/mmwr.mm6533e1
5. Torio C M, Andrews R M. STATISTICAL BRIEF #160 National Inpatient Hospital Costs: The Most. Healthc Cost Util Proj Stat Br #160 Natl Inpatient Hosp Costs Most Expens Cond by Payer, 2011. 2013; 31(1):1-12.
6. Buehler S S, Madison B, Snyder S R, et al. Effectiveness of practices to increase timeliness of providing targeted therapy for inpatients with bloodstream infections: A laboratory medicine best practices systematic review and meta-analysis. Clin Microbiol Rev. 2015; 29(1):59-103. doi:10.1128/CMR.00053-14
7. Kiser C, Nawab U, McKenna K, Aghai Z H. Role of guidelines on length of therapy in chorioamnionitis and neonatal sepsis. Pediatrics. 2014; 133(6):992-998. doi:10.1542/peds.2013-2927
8. Storro O, Avershina E, Rudi K. Diversity of intestinal microbiota in infancy and the risk of allergic disease in childhood. Curr Opin Allergy Clin Immunol. 2013; 13(3):257-262. doi:10.1097/ACI.0b013e328360968b
9. Saari A, Virta L J, Sankilampi U, Dunkel L, Saxen H. Antibiotic exposure in infancy and risk of being overweight in the first 24 months of life. Pediatrics. 2015; 135(4):617-626. doi:10.1542/peds.2014-3407
10. Seymour C W, Gesten F, Prescott H C, et al. Time to treatment and mortality during mandated emergency care for sepsis. N Engl J Med. 2017; 376(23):2235-2244. doi:10.1056/NEJMoa1703058
11. Kumar A, Roberts D, Wood K E, et al. Duration of hypotension before initiation of effective antimicrobial therapy is the critical determinant of survival in human septic shock. Crit Care Med. 2006; 34(6):1589-1596. doi:10.1097/01.CCM.0000217961.75225.E9
12. Kumar A, Ellis P, Arabi Y, et al. Initiation of inappropriate antimicrobial therapy results in a fivefold reduction of survival in human septic shock. Chest. 2009; 136(5):1237-1248. doi:10.1378/chest.09-0087
13. Morrell M, Fraser V J, Kollef M H. Delaying the empiric treatment of candida bloodstream infection until positive blood culture results are obtained: a potential risk factor for hospital mortality. Antimicrob Agents Chemother. 2005; 49(9):3640-3645. doi:10.1128/AAC.49.9.3640-3645.2005
14. Garey K W, Rege M, Pai M P, et al. Time to initiation of fluconazole therapy impacts mortality in patients with candidemia: a multi-institutional study. Clin Infect Dis. 2006; 43(1):25-31. doi:10.1086/504810
15. Fauci A S, Marston lary D. The perpetual challenge of antimicrobial resistance. JAMA. 2014; 311(18):1853-1854. doi:10.1001/jama.2014.2465
16. Ventola C L. The Antibiotic Resistance: part 1: causes and threats. P T. 2015; 40(4):277-283.
17. Reddy B, Hassan U, Seymour C, et al. Point-of-care sensors for the management of sepsis. Nat Biomed Eng. 2018; 2(9):640-648. doi:10.1038/s41551-018-0288-9
18. Dellinger R P, Levy M M, Carlet J M, et al. Surviving Sepsis Campaign: International guidelines for management of severe sepsis and septic shock: 2008. Intensive Care Med. 2008; 34(1):17-60. doi:10.1007/s00134-007-0934-2
19. Kollef M H, Sherman G, Ward S, Fraser V J. Inadequate antimicrobial treatment of infections: A risk factor for hospital mortality among critically III patients. Chest. 1999; 115(2):462-474. doi:10.1378/chest.115.2.462
20. Gamacho-Montero J, Garcia-Garmendia J L, Barrero-Almodovar A, Jimenez-Jimenez F J, Perez-Paredes C, Ortiz-Leyba C. Impact of adequate empirical antibiotic therapy on the outcome of patients admitted to the intensive care unit with sepsis. Crit Care Med. 2003; 31(12):2742-2751. doi:10.1097/01.CCM.0000098031.24329.10
21. Valles J, Rello J, Ochagavia A, Garnacho J, Alcali M A. Community-acquired bloodstream infection in critically ill adult patients: impact of shock and inappropriate antibiotic therapy on survival. Chest. 2003; 123(5):1615-1624. doi:10.1378/chest.123.5.1615
22. T2biosystems. Clinical Trial Data Supporting FDA Clearance of the T2Bacteria® Panel Versus Blood Culture for the Diagnosis of Bacteremia The T2Bacteria Panel Prospective Multicenter Pivotal Clinical Trial Demonstrated Distinct Advantages of T2Bacteria over Blood Cultur.
23. Fenollar F, Raoult D. Molecular diagnosis of bloodstream infections caused by non-cultivable bacteria. Int J Antimicrob Agents. 2007; 30(SUPPL. 1):7-15. doi:10.1016/j.ijantimicag.2007.06.024
24. Blackburn R M, Muller-Pebody B, Planche T, et al. Neonatal sepsis—many blood samples, few positive cultures: implications for improving antibiotic prescribing. Arch Dis Child Fetal Neonatal Ed. 2012; 97(6):F487-8. doi:10.1136/archdischild-2012-302261
25. Ottolini M C, Lundgren K, Mirkinson L J, Cason S, Ottolini M G. Utility of complete blood count and blood culture screening to diagnose neonatal sepsis in the asymptomatic at risk newborn. Pediatr Infect Dis J. 2003; 22(5):430-434. doi:10.1097/01.inf.0000068206.11303.dd
26. Lin L, Nonejuie P, Munguia J, et al. Azithromycin Synergizes with Cationic Antimicrobial Peptides to Exert Bactericidal and Therapeutic Activity Against Highly Multidrug-Resistant Gram-Negative Bacterial Pathogens. EBioMedicine. 2015; 2(7):690-698. doi:10.1016/j.ebiom.2015.05.021
27. Waites K B, Canupp K C. Evaluation of BacT/ALERT system for detection of Mycoplasma hominis in simulated blood cultures. J Clin Microbiol. 2001; 39(12):4328-4331. doi:10.1128/JCM.39.12.4328-4331.2001
28. Nawrot U, Kowalska-Krochmal B, Sulik-Tyszka B, et al. Evaluation of blood culture media for the detection of fungi. Eur J Clin Microbiol Infect Dis. 2014; 34(1):161-167. doi:10.1007/s10096-014-2218-4
29. Zadroga R, Williams D N, Gottschall R, et al. Comparison of 2 blood culture media shows significant differences in bacterial recovery for patients on antimicrobial therapy. Clin Infect Dis. 2013; 56(6):790-797. doi:10.1093/cid/cis1021
30. Bhattacharyya A, Klapperich C M. Thermoplastic microfluidic device for on-chip purification of nucleic acids for disposable diagnostics. Anal Chem. 2006; 78(3):788-792. doi:10.1021/ac051449j
31. Tan S C, Yiap B C. DNA, RNA, and protein extraction: The past and the present. J Biomed Biotechnol. 2009; 2009. doi:10.1155/2009/574398
32. Qiagen. QIAprep Miniprep Handbook. QIAprep Miniprep Handb. 2012; (May):1-44. doi:10.1111/j.1468-1331.2009.02749.x
33. Katevatis C, Fan A, Klapperich C M. Low concentration DNA extraction and recovery using a silica solid phase. PLoS One. 2017; 12(5):1-14. doi:10.1371/journal.pone.0176848
34. Wojewoda C M, Sercia L, Navas M, et al. Evaluation of the verigene gram-positive blood culture nucleic acid test for rapid detection of bacteria and resistance determinants. J Clin Microbiol. 2013; 51(7):2072-2076. doi:10.1128/JCM.00831-13
35. FilmArray Blood Culture Identification Panel.
36. Lau A, Sorrell T C, Chen S, Stanley K, Iredell J, Halliday C. Multiplex tandem PCR: a novel platform for rapid detection and identification of fungal pathogens from blood culture specimens. J Clin Microbiol. 2008; 46(9):3021-3027. doi:10.1128/JCM.00689-08
37. De Baere T, Claeys G, Swinne D, et al. Identification of cultured isolates of clinically important yeast species using fluorescent fragment length analysis of the amplified internally transcribed rRNA spacer 2 region (ITS2). BMC Microbiol. 2002; 2:21.
38. Dierkes C, Ehrenstein B, Siebig S, Linde H-J, Reischl U, Salzberger B. Clinical impact of a commercially available multiplex PCR system for rapid detection of pathogens in patients with presumed sepsis. BMC Infect Dis. 2009; 9:126. doi:10.1186/1471-2334-9-126
39. Einsele H, Hebart H, Roller G, et al. Detection and identification of fungal pathogens in blood by using molecular probes. J Clin Microbiol. 1997; 35(6):1353-1360.
40. T2Bacteria Panel—T2 Biosystems.
41. Dietzman D E, Fischer G W, Schoenknecht F D. Neonatal Escherichia coli septicemia—bacterial counts in blood. J Pediatr. 1974; 85(1):128-130. doi:10.1016/s0022-3476(74)80308-2
42. Kellogg J A, Ferrentino F L, Goodstein M H, Liss J, Shapiro S L, Bankert D A. Frequency of low level bacteremia in infants from birth to two months of age. Pediatr Infect Dis J. 1997; 16(4):381-385. doi:10.1097/00006454-199704000-00009
43. Ngo-Giang-Huong N, Khamduang W, Leurent B, et al. Early HIV-I diagnosis using in-house real-time PCR amplification on dried blood spots for infants in remote and resource-limited settings. J Acquir Immune Defic Syndr. 2008; 49(5):465-471. doi:10.1097/QAI.0b013e31818e2531
44. Gupta B P, Jayasuryan N, Jameel S. Direct detection of hepatitis B virus from dried blood spots by polymerase chain reaction amplification. J Clin Microbiol. 1992; 30(8):1913-1916. doi:10.1128/jcm.30.8.1913-1916.1992
45. Saavedra-Matiz C A, Isabelle J T, Biski C K, et al. Cost-effective and scalable DNA extraction method from dried blood spots. Clin Chem. 2013; 59(7):1045-1051. doi:10.1373/clinchem.2012.198945
46. Mostafa A, Ganguli A, Berger J, et al. Culture-free biphasic approach for sensitive detection of Escherichia coli O157:H7 from beef samples. Biotechnol Bioeng. 2021; 118(11):4516-4529. doi:10.1002/bit.27920
47. Ganguli A, Ornob A, Spegazzini N, et al. Pixelated spatial gene expression analysis from tissue. Nat Commun. 2018; 9(1):1-9. doi:10.1038/s41467-017-02623-9
48. Xu Z, Li L, Chu J, et al. Development and application of loop-mediated isothermal amplification assays on rapid detection of various types of staphylococci strains. Food Res Int. 2012; 47(2):166-173. doi:10.1016/j.foodres.2011.04.042
49. Hill J, Beriwal S, Chandra I, et al. Loop-mediated isothermal amplification assay for rapid detection of common strains of Escherichia coli. J Clin Microbiol. 2008; 46(8):2800-2804. doi:10.1128/JCM.00152-08
50. Englen M D, Kelley L C. A rapid DNA isolation procedure for the identification of Campylobacter jejuni by the polymerase chain reaction. Lett Appl Microbiol. 2000; 31(6):421-426. doi:10.1046/j.1365-2672.2000.00841.x
51. Liu Y, Schulze-Makuch D, de Vera J P, et al. The development of an effective bacterial single-cell lysis method suitable for whole genome amplification in microfluidic platforms. Micromachines. 2018; 9(8). doi:10.3390/mi9080367
52. Greenwood C, Morrow A L, Lagomarcino A J, et al. Early empiric antibiotic use in preterm infants is associated with lower bacterial diversity and higher relative abundance of enterobacter. J Pediatr. 2014; 165(1):23-29. doi:10.1016/j.jpeds.2014.01.010
53. Kreger B E, Craven D E, Carling P C, McCabe W R. Gram-negative bacteremia. III. Reassessment of etiology, epidemiology and ecology in 612 patients. Am J Med. 1980; 68(3):332-343. doi:10.1016/0002-9343(80)90101-1
54. Bacconi A, Richmond G S, Baroldi M A, et al. Improved sensitivity for molecular detection of bacterial and candida infections in blood. J Clin Microbiol. 2014; 52(9):3164-3174. doi:10.1128/JCM.00801-14
55. Opota O, Jaton K, Greub G. Microbial diagnosis of bloodstream infection: Towards molecular diagnosis directly from blood. Clin Microbiol Infect. 2015; 21(4):323-331. doi:10.1016/j.cmi.2015.02.005
56. Magro L, Escadafal C, Gameret P, et al. Paper microfluidics for nucleic acid amplification testing (NAAT) of infectious diseases. Lab Chip. 2017; 17(14):2347-2371. doi:10.1039/c71c00013 h
57. Pien B C, Sundaram P, Raoof N, et al. The clinical and prognostic importance of positive blood cultures in adults. Am J Med. 2010; 123(9):819-828. doi:10.1016/j.amjmed.2010.03.021
58. Garey K W, Rege M, Pai M P, et al. Time to Initiation of Fluconazole Therapy Impacts Mortality in Patients with Candidemia: A Multi-Institutional Study. Clin Infect Dis. 2006; 43(1):25-31. doi:10.1086/504810
59. Spanakis E K, Aperis G, Mylonakis E. Reviews Of Anti-infective Agents: New Agents for the Treatment of Fungal Infections: Clinical Efficacy and Gaps in Coverage. Clin Infect Dis. 2006; 43(8):1060-1068. doi:10.1086/507891
60. Pfaller M A, Diekema D J. Epidemiology of invasive candidiasis: A persistent public health problem. Clin Microbiol Rev. 2007; 20(1):133-163. doi:10.1128/CMR.00029-06
61. Chaffin W L. Candida albicans Cell Wall Proteins. Microbiol Mol Biol Rev. 2008; 72(3):495-544. doi:10.1128/mmbr.00032-07
62. Thompson D S, Carlisle P L, Kadosh D. Coevolution of morphology and virulence in Candida species. Eukaryot Cell. 2011; 10(9):1173-1182. doi:10.1128/E C.05085-11
63. Rodriguez A, Vaneechoutte M. Comparison of the efficiency of different cell lysis methods and different commercial methods for RNA extraction from Candida albicans stored in RNAlater. BMC Microbiol. 2019; 19(1):94. doi:10.1186/s12866-019-1473-z
64. Kasahara K, Ishikawa H, Sato S, Shimakawa Y, Watanabe K. Development of multiplex loop-mediated isothermal amplification assays to detect medically important yeasts in dairy products. FEMS Microbiol Lett. Published online July 2014:n/a-n/a. doi:10.1111/1574-6968.12512
65. Liao C H, Shollenberger L M. Survivability and long-term preservation of bacteria in water and in phosphate-buffered saline. Lett Appl Microbiol. 2003; 37(1):45-50. doi:10.1046/j.1472-765X.2003.01345.x
66. Neely L A, Audeh M, Phung N A, et al. T2 magnetic resonance enables nanoparticle-mediated rapid detection of candidemia in whole blood. Sci Transl Med. 2013; 5(182):1-9. doi:10.1126/scitranslmed.3005377
67. Lukacs G, Haggie P, Seksek O, . . . D L-J of biological, 2000 undefined. Size-dependent DNA mobility in cytoplasm and nucleus. ASBMB.
68. Weller H G, Tabor G, Jasak H, Fureby C. A tensorial approach to computational continuum mechanics using object-oriented techniques. Comput Phys. 1998; 12(6):620. doi:10.1063/1.168744
69. Kim O V, Litvinov R I, Chen J, Chen D Z, Weisel J W, Alber M S. Compression-induced structural and mechanical changes of fibrin-collagen composites. Matrix Biol. 2017; 60-61:141-156. doi:10.1016/j.matbio.2016.10.007
70. van Dooren G G, Kennedy A T, McFadden G I. The Use and Abuse of Heme in Apicomplexan Parasites. Antioxid Redox Signal. 2012; 17(4):634-656. doi:10.1089/ars.2012.4539
71. Koirenf L, Obomik M, Lukes J. Make It, Take It, or Leave It: Heme Metabolism of Parasites. Knoll L J, ed. PLoS Pathog. 2013; 9(1):e1003088. doi:10.1371/journal.ppat.1003088
72. Tomita N, Mon Y, Kanda H, Notomi T. Loop-mediated isothermal amplification (LAMP) of gene sequences and simple visual detection of products. Nat Protoc. 2008; 3(5):877-882. doi:10.1038/nprot.2008.57
73. Goto M, Honda E, Ogura A, Nomoto A, Hanaki K I. Colorimetric detection of loop-mediated isothermal amplification reaction by using hydroxy naphthol blue. Biotechniques. 2009; 46(3):167-172. doi:10.2144/000113072

### Example 6: A Blood Drying Process for DNA Amplification

The presence of numerous inhibitors in blood makes their use in nucleic acid amplification techniques difficult. Current methods for extracting and purifying pathogenic DNA from blood involve removal of inhibitors, resulting in low and inconsistent DNA recovery rates. To address this issue, we have developed a biphasic method that simultaneously achieves inhibitor inactivation and DNA amplification without the need for a purification step. Inhibitors are physically trapped in the solid-phase dried blood matrix by blood drying, while amplification reagents can move into the solid nano-porous dried blood and initiate the amplification. We demonstrate that the biphasic method has significant improvement in detection limits for bacteria such as E. coli, MRSA, MSSA using LAMP and RPA. Several factors, such as drying time, sample volume, and material properties were characterized to increase sensitivity and expand the application of the biphasic assay to blood diagnostics. With further automation, this biphasic technique has the potential to be used as a diagnostic platform for the detection of pathogens eliminating lengthy culture steps.

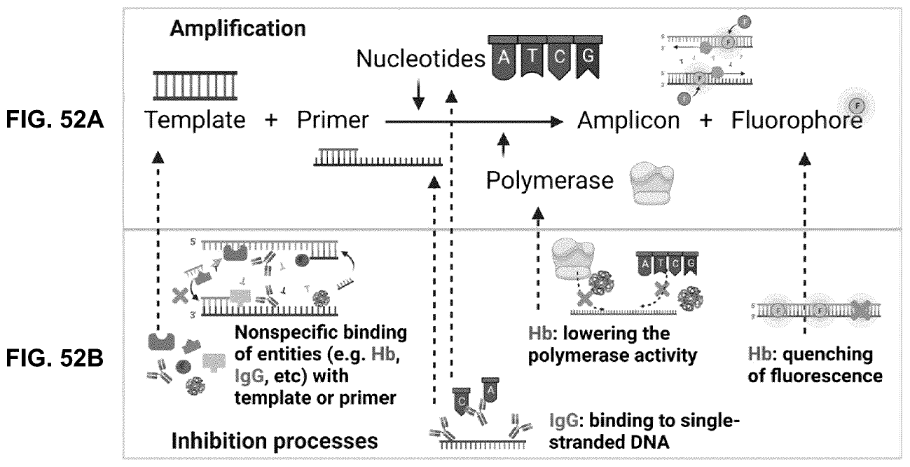

In the context of human health, blood serves a critical function and provides valuable information regarding the physiological state of an individual. Blood analysis is a useful tool for detecting a range of ailments, including genetic disorders, microbial and viral infections, as well as cancer.^{[1-4]} The standard method for nucleic acid detection from blood involves extraction and purification of nucleic acid followed by amplification techniques such as polymerase chain reaction (PCR). Amplification is initiated by the binding of primers to the template, and polymerase extends the template length by adding the nucleotides, resulting in numerous amplicons (FIG. 52A). Fluorescent dye intercalating the double strand DNA can be used to detect the presence of generated amplicons. However, as the target nucleic acid is present in low concentration and is surrounded by numerous inhibitor components in blood that may bind to the target, primers, or polymerase, the quality of the nucleic acid extraction and purification process has critical impact on the success of downstream nucleic amplification techniques (FIG. 52B). Thus, appropriate pre-treatment and nucleic acid extraction and treatment procedures are important for obtaining accurate and reliable results.

Despite the importance of nucleic acid extraction and purification in the blood analysis process, the convetional methods have several drawbacks. First, they are expensive, time-consuming, and labor intensive.^{[5,6]} Moreover, there is a risk of cross-contamination during the multiple steps involved in sample processing.^{[7]} Very importantly, removal of PCR inhibitors is challenging due to the high number of precise manual steps required.^{[1]} The most significant issue is the loss of target nucleic acid itself during the current purification and extraction processes. This is due to inherent loss mechanisms, and the inefficiency of binding and unbinding of the negatively charged nucleic acid to positively charged silica surfaces, hence limiting the efficiency of capturing and retaining a few copies of target pathogenic DNA against millions of copies of human genomic DNA. Consequently, the starting concentration for target nucleic acid in blood is limited to higher than 1 μg. This poses a significant challenge for the detection of microbial or viral infections in blood,^{[8]} including for initial portion of the infection time-course, where the pathogen load may be relatively low.

Considering the challenges associated with traditional blood treatment methods, there is a need to develop a new blood treatment module that simplifies pre-treatment steps prior to nucleic acid amplification testing (NAAT) and bypasses extraction and purification. The two primary obstacles to overcome are the reduction of inhibitor levels in blood and the retention of target DNA molecules without loss. Blood contains numerous inhibitors that bind nonspecifically to key components of amplification reactions, reducing the efficiency of the reaction. Hemoglobin and immunoglobulin G (IgG) are two of the main inhibitors found in blood, and can lower the activity of polymerase or even inactivate it, bind with cofactors, or degrade the target or primers (FIG. 52B).^{[6,9,10]} Additionally, hemoglobin can interfere with fluorescence readout and make amplification detection difficult by the absorption and scattering of the light.^{[9]}

We have demonstrated highly sensitive detection (limit of detection ˜1 CFU/mL) of bloodstream infections caused by bacteria, including Methicillin-Sensitive Staphylococcus aureus (MSSA), Methicillin-resistant Staphylococcus aureus (MRSA), and Escherichia coli (E. coli), without blood culture, and extraction and purification.^{[11]} We utilized blood to create a structure within the PCR tubes by heating the blood to result in a dried blood matrix. Additionally, the use of loop-mediated isothermal amplification (LAMP) with a robust Bst polymerase results in amplification that is not affected by inhibitors.^{[12]} The approach is termed as ‘biphasic reaction’ since it consists of two phases: a solid phase of the dried blood matrix and a liquid phase of supernatant also allowing clear fluorescence detection.^{[13]} The developed assay was shown to be highly sensitive and robust to inhibitors, but its mechanism has not yet been thoroughly investigated. See also U.S. Pat. Pub. No. 2020/0263244 (which is specifically incorporated by reference herein, including for the systems and methods of detecting biomarkers from fluid samples).

In this example, the mechanism of this heating induced biphasic reaction is investigated, with a focus on understanding the inhibitory processes and ways to improve assay sensitivity of the reaction. Inhibitor levels were characterized in the liquid phase before and after biphasic thermal treatment using colorimetric or enzyme-linked immunoassay (ELISA) measurements. To determine the existence of inhibitors within the dried blood matrix (solid phase), liquid extraction surface analysis (LESA) coupled mass spectrometry (MS) was utilized. Additionally, various parameters, including drying time, diffusion distance, and material properties, were investigated to establish the optimal conditions for improving assay sensitivity when using the biphasic protocol. Lastly, we demonstrated a new application of the biphasic reaction in recombinase polymerase amplification (RPA), expanding the potential use of the technique in general nucleic acid amplification techniques.

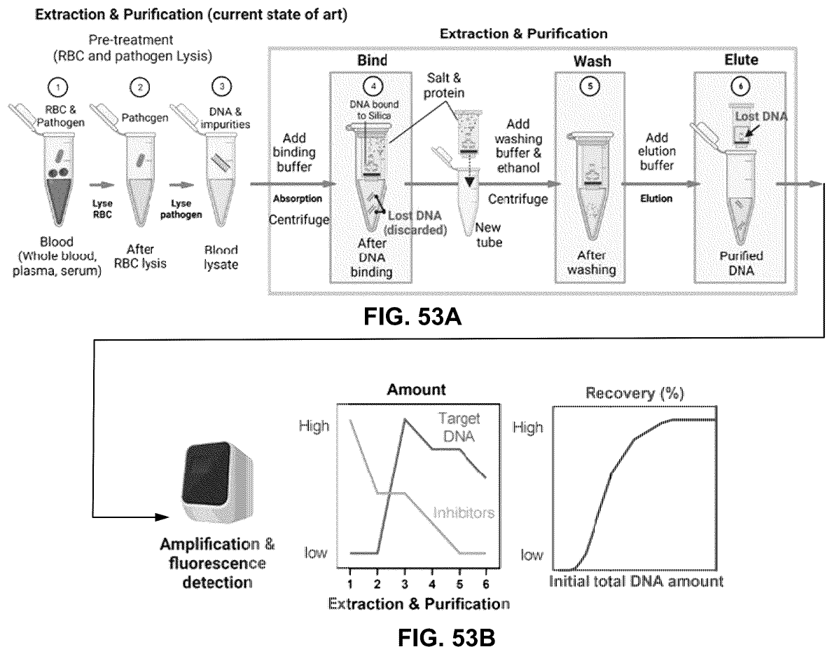

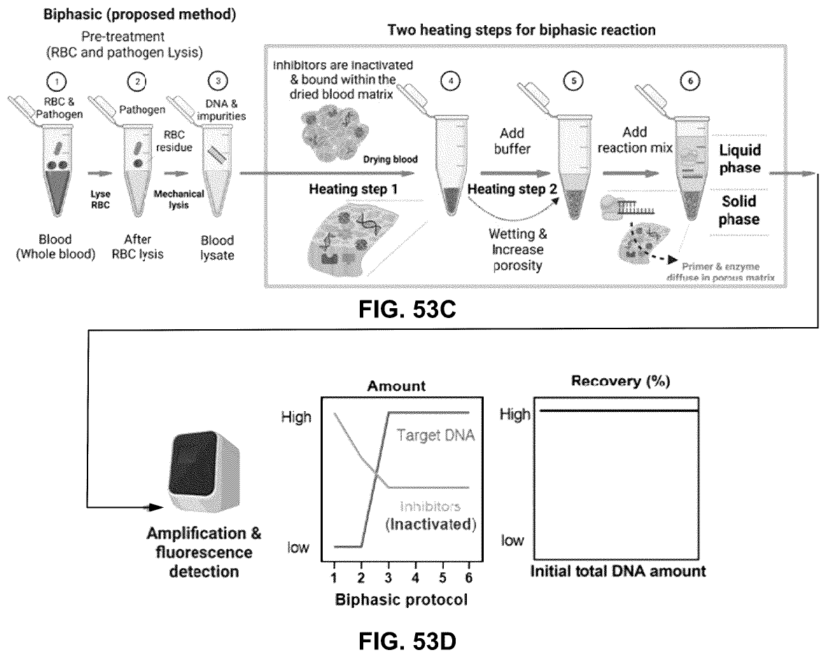

Biphasic reaction: FIGS. 53A-53D introduce the current state of the art in extraction and purification method in comparison to the new biphasic assay. During each step, the presence of the two primary components of interests, the inhibitors and target DNA, are depicted in FIG. 53A and FIG. 53C, while their relative level during the process is shown in FIGS. 53B and 53D. Nucleic acid extraction and purification starts with the pre-treatment steps such as red blood cell (RBC) lysis and pathogen lysis (FIG. 52A, step 1-3). The starting material could be either whole blood or plasma and serum if the samples went through centrifugation (step 1). Ammonium-Chloride-Potassium (ACK) lysis buffer is employed for selective RBC lysis, using a volume 10-20 times greater than that of the blood sample to ensure complete removal of the RBCs (step 2). Chemical lysis buffers are commonly utilized for pathogen lysis (step 3), in which detergents and enzymes disrupt the cell wall of the pathogens, leading to the release of targeted nucleic acid from within the pathogen. Following the lysis of the pathogen, the sample comprises elevated levels of targeted nucleic acid alongside impurities (FIG. 53B). During the centrifugation, there is absorption of the negatively charged DNA onto the surface of purification materials, such as positively charged silica (step 4). A fraction of unbound DNA may be lost during the centrifugation step, as the bottom tube is discarded. Furthermore, during the purification process, various other impurities, including proteins, polysaccharides, and salts, may adhere to the purification materials and need to be eliminated through the washing step (step 5). Finally, an elution step is performed to generate the ultimate purified DNA sample, which can subsequently be utilized for amplification and fluorescence detection. Lost DNA during the process is highlighted in red (step 4 and 6). FIG. 53B conceptually illustrates the variations in inhibitor and target DNA levels that could occur during the extraction and purification process. These changes suggest that the method results in the reduction of inhibitors, albeit at the expense of inevitable DNA loss. This limitation may affect the efficiency of the extraction and purification process, particularly when starting with low DNA concentrations, as it may result in insufficient purified DNA at the end of the process. Consequently, the recovery rate of DNA is dependent on the initial total DNA amount especially at low DNA copies.

Conversely, the biphasic method utilizes a simple process of drying the blood sample (heating step 1) which results in inactivation of inhibitors, followed by a heating step 2 to enhance the sensitivity of the assay (FIG. 53C). This method commences with the use of whole blood as the specimen (from step 1 to step 4 directly) or, alternatively, can start with the blood lysate by RBC lysis (step 2) and mechanical lysis of bacteria (step 3) to obtain DNA readily available for downstream analysis. In the biphasic method, the RBC lysis process involves the use of a 1:1 or 1:2 ratio of whole blood to ACK lysis buffer to partially remove RBC rather than a ratio of 1:10 or 1:20 whole blood to ACK lysis buffer.^{[11]} This approach is employed to maintain the stability of the dried blood matrix by leaving a portion of RBC in the sample, which helps to facilitate the formation of a solid structure during the drying process.

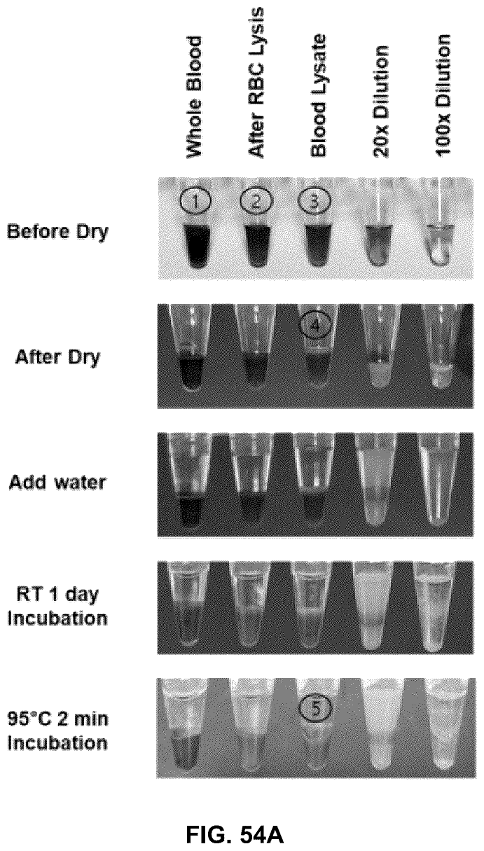

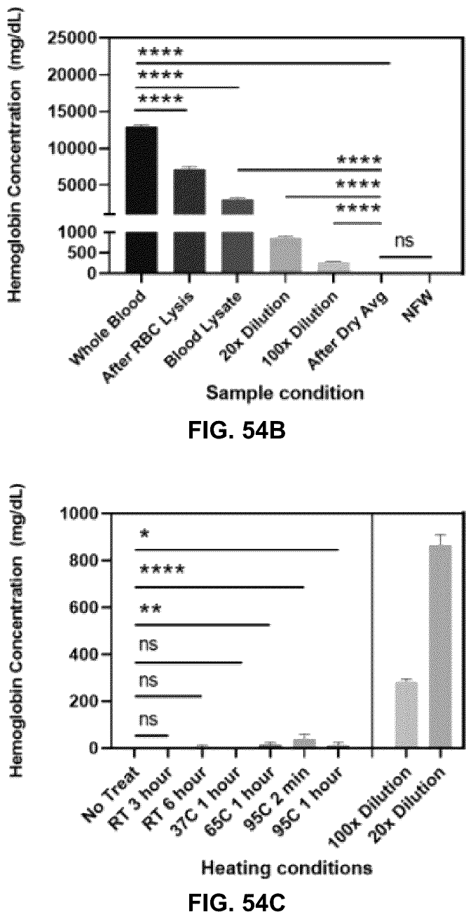

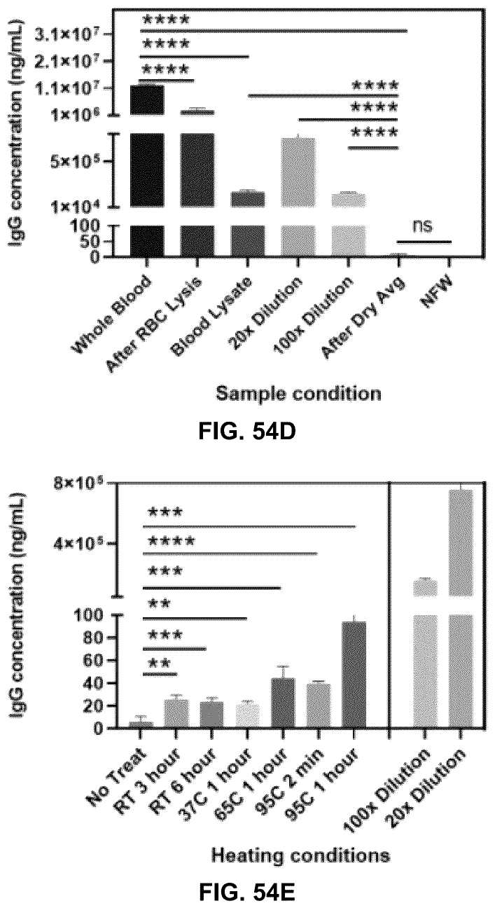

It should be noted that complete removal of RBC can lead to an unstable matrix structure, as shown in FIG. 54A for the ‘100× Dilution’. After completing the pre-treatment process (steps 1-3), the sample is ready to undergo the biphasic protocol. The optimal temperature and duration of heat for drying (heating step 1) the blood sample may vary based on the sample volume used. We utilize a volume of 30 μL and dried it at 95° C. for 10 minutes. During the blood drying (step 4), we postulate that the amplification inhibitors are inactivated and become bound within the dried blood matrix. Meanwhile, the concentration of DNA in the blood is maintained, as no additional binding and elution steps are required, unlike in state of the art extraction and purification processes. The resultant dried blood matrix could be effective in neutralizing inhibitors; however, it is suboptimal for nucleic acid amplification and detection, primarily due to its thick and nonporous structure that arises from the compaction of the blood during coagulation. To overcome this limitation, we introduced a buffer and conducted the second important step in the biphasic reaction protocol, namely a heating step 2 (95° C. for 2 minutes) (step 5), to increase the porosity and wetting of the dried blood matrix. Before the heating step 2, we refrained from adding primers and polymerase to the buffer solution, as the polymerase, especially could be degraded at the high temperature conditions used in heating step 2. Following heating step 2 (step 5), a reaction mix comprising of primers and polymerase is added (step 6). The tube now contains two phases, namely a liquid and a solid phase (FIG. 53C, step 6). The solid phase contains the inactivated inhibitors and target DNA bound within the dried blood matrix. On the other hand, the liquid phase now contains polymerase and primers, which can diffuse into the porous dried blood matrix, and initiate amplification upon binding to the target DNA. After amplification, the resulting amplicons diffuse out from the solid phase into the liquid phase. The fluorescent intercalating dye provides a signal from the clear supernatant measurements without interference from the red components in blood. Thus, by physically separating the two phases and detecting only the liquid phase for amplification and fluorescence measurement, we can increase the signal to noise. FIG. 53D demonstrates the effect of the biphasic process on the target DNA levels. The target DNA level remains preserved during the biphasic process (steps 3-6), while inhibitor levels remain constant at a steady state. However, the inhibitors exist in an inactivated form and do not participate in the amplification reaction, resulting in a high recovery rate and amplification of the target DNA, irrespective of the initial total DNA amount. This is crucial for detecting low-concentration pathogens, such as those involved in sepsis diagnosis.

Characterization of the inhibitor levels in liquid phase: To assess whether inhibitors are truly inactivated following blood drying and if they are not subsequently solubilized into the liquid phase, we undertook a characterization of two significant amplification inhibitors: hemoglobin and immunoglobulin G (IgG). FIG. 54A illustrates the analysis of the blood sample and its derivatives associated with the biphasic protocol. The figure is organized into five groups of samples presented in columns, which include whole blood, blood after RBC lysis, blood lysate after mechanical lysis, and two control groups: blood that has been diluted with water by 20 times and by 100 times. Each group is further divided into rows that represent the process of sample preparation, including drying, addition of water, and incubation either at room temperature for one day or at 95° C. for 2 minutes (heating step 2 condition). The numbers (1-5) in the image correspond to the individual steps outlined in the biphasic protocol illustrated in FIG. 53C. To determine if the samples can generate a stable dried blood matrix after undergoing the blood drying process, a color change was observed after adding water (row 3 in FIG. 54A). It was evident that samples 1-3 produced a stable structure that prevented the solid phase from being resuspended into the liquid phase, in contrast to the 20× and 100× diluted blood samples. Furthermore, in the case of the 20× diluted blood sample, once incubated either at room temperature or at 95° C., the solid phase is solubilized into the liquid phase, which ultimately results in the interruption of fluorescence readings.

To obtain a quantitative assessment of inhibitor levels in accordance with the biphasic protocol, a colorimetric kit for hemoglobin and an enzyme-linked immunosorbent assay (ELISA) for immunoglobulin G (IgG) were employed and are presented in FIGS. 54B-54E. After allowing 10 minutes of incubation to enable the solubilization of inhibitors into the supernatant, measurements were conducted on the supernatant of each sample based on the vendor's protocol. FIG. 54B displays the level of hemoglobin prior to and after blood drying. Distinctive reductions in hemoglobin levels were observed during each pre-treatment step (steps 1-3, as shown in FIG. 53C) as the samples were processed from the initial ‘Whole Blood’ state to ‘after RBC Lysis’, and finally to ‘Blood Lysate’. The concentration of hemoglobin in the blood lysate was still greater than that of the 20× diluted blood sample, indicating that the pre-treatment procedure did not completely remove hemoglobin. The phrase “After Dry Avg” in the x-axis refers to the hemoglobin level of three samples (Whole Blood, After RBC lysis, Blood Lysate) following blood drying. All three samples displayed hemoglobin levels that were close to zero after drying, with no significant variation in comparison to nuclease-free water (NFW). Subsequently, water was added to the samples (sample 1-3), which were then incubated under varying conditions to measure the hemoglobin level and to verify whether hemoglobin physically trapped within the dried blood matrix was indeed inactivated (FIG. 54C). Direct samples were used for measurement for 20× and 100× diluted blood samples. Incubation at room temperature and 37° C. did not show any significant difference in comparison to the negative control sample (i.e., no treatment). Although incubation at higher temperatures utilized for loop-mediated isothermal amplification (LAMP) at 65° C. and heating step 2 at 95° C. showed a notable variation in comparison to the negative control sample, their hemoglobin levels remained significantly lower when compared to the 100× diluted blood sample. Similar to the hemoglobin analysis, characterizations were conducted on IgG and are displayed in FIGS. 54D-54E. A considerably larger proportion of IgG was removed for the blood lysate (i.e., comparable to a 100× dilution), but similar patterns of inactivation following drying were observed (i.e., the “After Dry Avg” was near zero). In contrast, incubations steps showed some dissolve of IgG into liquid phase compared to the negative control (no treatment). However, considering the IgG concentration started from 107 ng/mL and decreased down to less than 100 ng/mL (difference in order of magnitude 105), its inactivation was considerably more pronounced than that of hemoglobin (from 10,000 mg/dL to 0 mg/dL). Also, this level of IgG dissolved in liquid phase was substantially lower than that of 100× diluted blood (˜105 ng/mL). A prior investigation of the inhibitory mechanism of blood components on real-time PCR demonstrated that the presence of 2.5% blood in the reaction resulted in a 45% quenching of fluorescence intensity, and an increase in hemoglobin concentration was found to have a linear correlation with a decrease in the number of positive reactions, starting at a concentration of 39 μM.^{[9]} Moreover, an IgG concentration of 190 μM (2.79×107 ng/mL) led to a delay in amplification time from 26 minutes to 32 minutes.^{[9]} It is noteworthy that the concentration of inhibitors in the supernatant of the biphasic reaction was significantly lower compared to the range that causes inhibition of amplification.

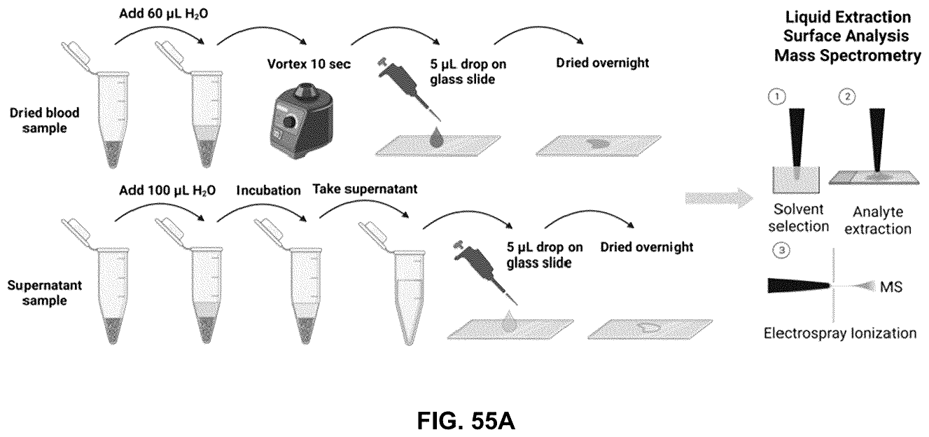

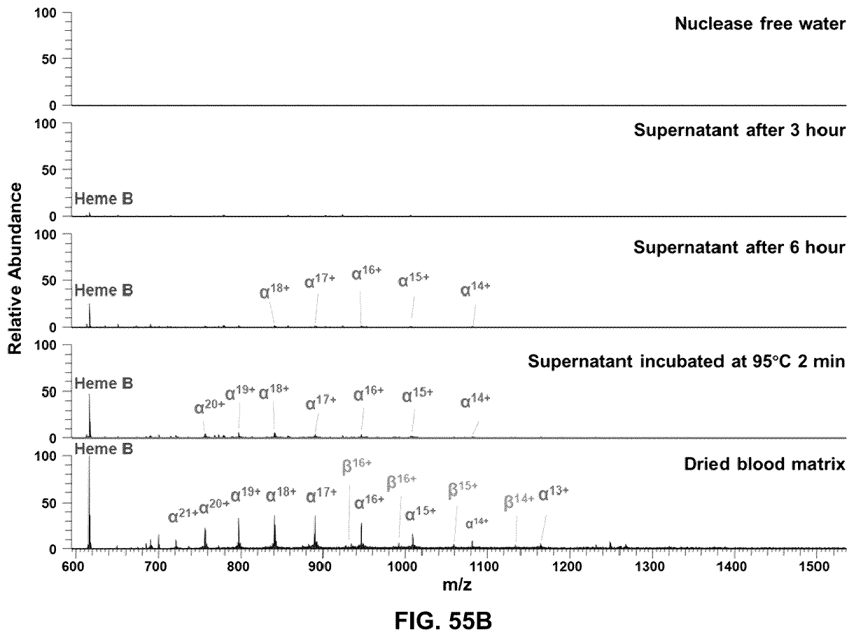

Characterization of the inhibitor levels in solid phase: To further investigate the existence of inhibitors within the dried blood matrix, liquid extraction surface analysis coupled mass spectrometry (LESA-MS) was used and results are presented in FIGS. 55A-55B. Protocol was adapted from previously described methods with modification.^{[14,15]}, FIG. 55A illustrates the two sample preparation protocols: one for the dried blood sample and the other for the supernatant. For the dried blood sample, after the dried blood matrix was suspended by vortexing, 5 μL of the mixture was utilized for measurement following overnight desiccation. The supernatant sample was utilized in a similar manner as in the inhibitor level measurement shown in FIGS. 54A-54E. FIG. 55B displays the relative abundance of heme B, α-globin and β-globin, which are subunits of hemoglobin, within the dried blood matrix under various conditions. Peaks were annotated based on a previous publication.^{[14]} As a result, a notably higher concentration of heme B, α-globin and β-globin peaks were observed in the dried blood matrix in comparison to the supernatant, highlighting that the majority of inhibitors actually exist within the dried blood matrix and were not present in the liquid phase.

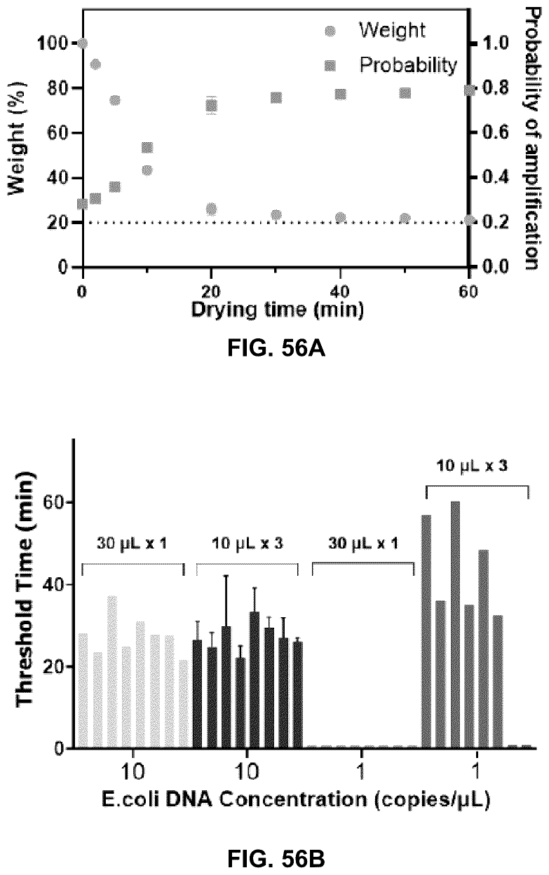

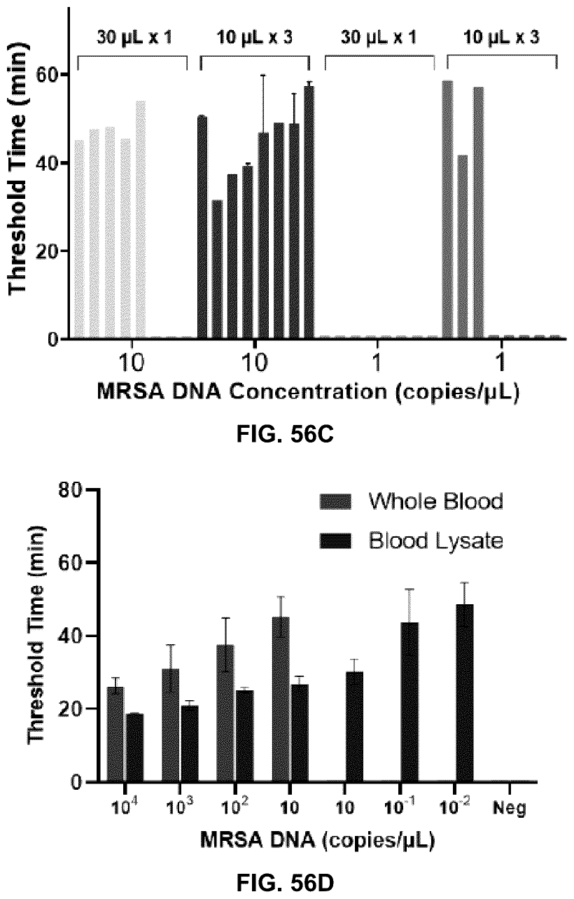

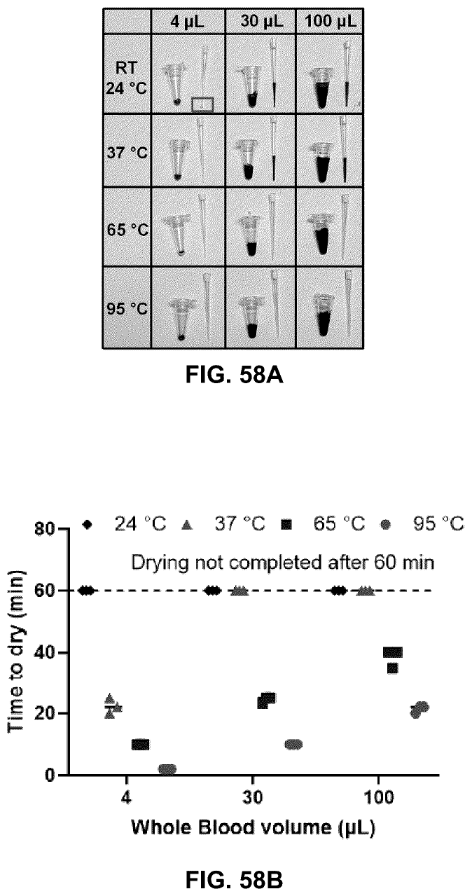

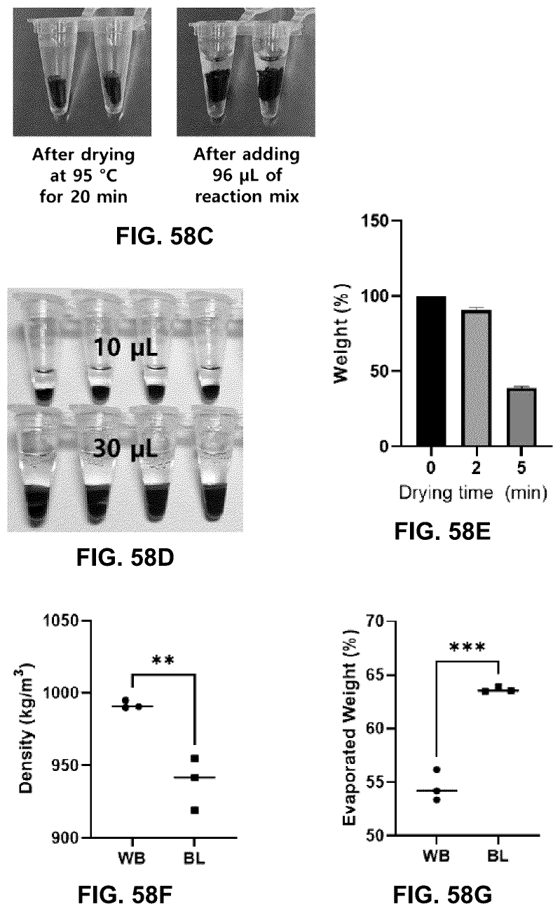

Highly Sensitive Biphasic LAMP Without Purification: For detection of DNA with high sensitivity, it is crucial for primers and polymerase to locate the target DNA easily and initiate the amplification faster. To identify such conditions, we performed the characterization on the biphasic protocol using three parameters: drying time, volume of the blood, and material properties. FIG. 56A depicts a decrease in sample weight over time as the sample dries, followed by an increase in the probability of amplification simply due to increasing concentration. We utilized a starting material volume of 30 μL, dried the sample at 95° C., and measured its weight at different time points. The liquid components of blood evaporated during drying, and the percentage of weight loss was recorded, with a starting weight of 100% and 40% remaining after 10 minutes of drying. After 20 minutes of drying, the weight remained constant and saturated at 20%. This weight loss can be attributed to the increased concentration per unit mass or per unit volume, as the target DNA concentration remains the same during the drying process. This highlights the potential improvement in the theoretical limit of detection (LOD) using this engineering design. For instance, we simulated the probability of amplification of 10 copies/30 μL concentration sample using Poisson statistics.^{[16,17]} The chance of detecting at least one genome copy increased from 28% when blood was not dried to 54% when blood was dried for 10 minutes, as the weight decreased by 44% and the DNA concentration increased proportionally to the evaporation rate. When the drying time exceeded 20 minutes, it caused the sample to contract and lose contact with the tube (FIG. 58C). This resulted in the entire dried blood matrix floating instead of remaining fixed at the bottom of the tube when buffer and reaction mix were added, causing the solid phase to intrude into the liquid phase and interfere with fluorescence reading. Thus, excessive drying time can adversely affect detection probability.

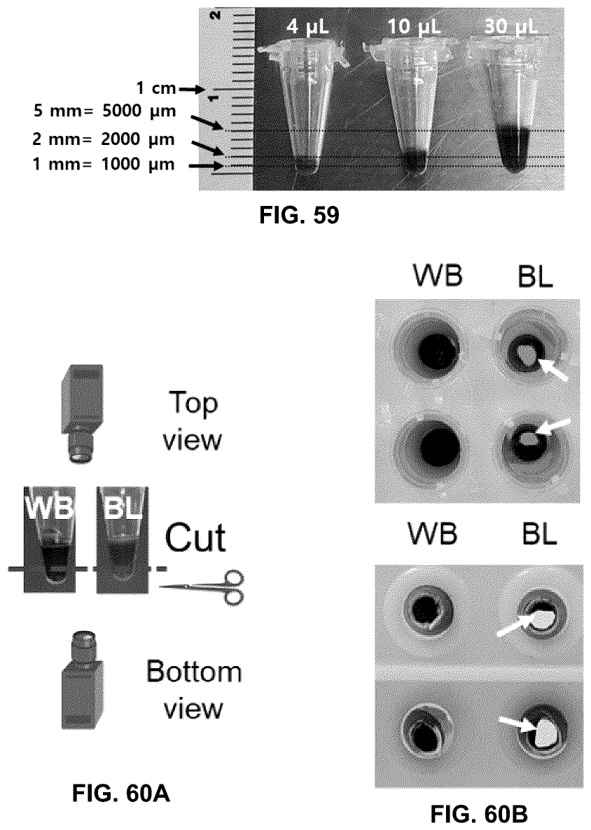

The sample volume related to the diffusion distance is the second major component for achieving better sensitivity. We used whole blood sample into two groups: one with 30 μL in one tube and the other with three tubes, each containing 10 μL. The 30 μL sample was dried at 95° C. for 10 minutes, resulting in a 57% weight loss, while the three tubes of 10 μL samples were dried at 95° C. for 5 minutes, resulting in a 61% weight loss (FIG. 58E). LAMP biphasic reaction was conducted for both samples, and the results were compared. We considered the 30 μL sample in one tube to be the same as the three tubes of 10 μL samples. The threshold time was characterized using whole blood spiked with either E. coli DNA (FIG. 56B) or MRSA DNA (FIG. 56C) at concentrations of 10 copies and 1 copy/30 μL. In the case of 10 copies/30 μL E. coli DNA spiked in whole blood (FIG. 56B), both sample types (30 μL×1 and 10 μL×3) showed all 8 replicates being amplified. However, when 1 copy/30 μL E. coli DNA was spiked in whole blood, only the 10 μL×3 sample group showed amplification (6 out of 8 replicates). Similar observations were made when using MRSA DNA (FIG. 56C). The 10 copies/30 μL MRSA DNA spiked in whole blood showed more amplification replicates when using the 10 μL×3 sample, while the 1 copy/30 μL MRSA DNA demonstrated amplification only for the 10 μL×3 sample (3 out of 8 replicates). The observed phenomenon can be explained due to changes in diffusion distance.^{[18]} Since the target DNA is retained during blood drying, the key factor is whether the amplification agents can reach the target DNA, which is mainly driven by diffusion within the dried blood matrix. In the case of the 30 μL sample in one tube, the diffusion length is longer. Consequently, primer and polymerase may not be able to reach the target DNA due to the dead-end microfluidic structure within the dried blood matrix. By dividing the sample into multiple tubes, the physical diffusion distance can be reduced. This distance change could also affect the diffusion distance of amplicons diffusing out to the supernatant for fluorescence detection once amplification is complete. Further explanation based on the estimation and simulation data is shown in FIG. 59. Additionally, using a smaller volume per well with much larger number of wells can reduce the time required for drying and potentially lowering the drying temperature. Application of this approach to larger volume used in clinical applications (˜5 mL) using automated division of sample, drying, distribution of amplification reagents, and fluorescence reading provide exciting opportunities for next steps. Furthermore, the ability of the primers to initiate and amplify the target DNA at a sufficient speed is another crucial parameter for a successful biphasic assay. The difference in the threshold time and the number of amplified replicates between E. coli and MRSA primer highlights the importance of the primer's ability for the amplification process.

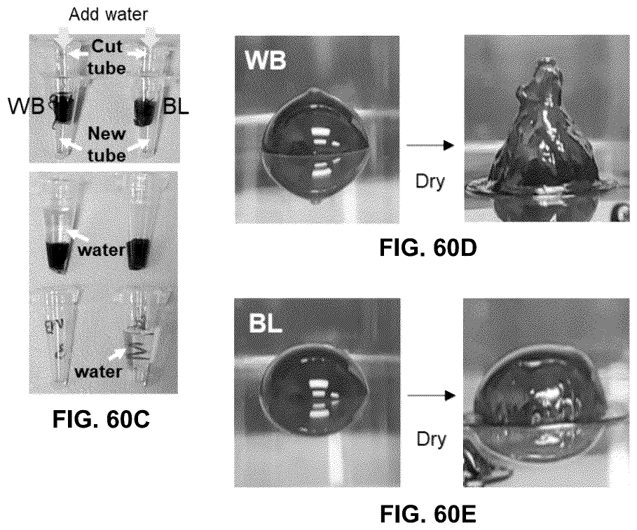

Finally, the material properties can significantly impact the sensitivity of the assay, as demonstrated in FIG. 56D. We compared the characteristics of whole blood and blood lysate, which are two primary starting materials for blood diagnostics. We first prepared the whole blood and blood lysate, spiked the MRSA DNA into each material, and tested them with biphasic LAMP reaction. Surprisingly, there was a three-magnitude order difference in LOD (10 copies/μL for whole blood and 0.01 copy/μL for blood lysate using a volume of 30 μL, FIG. 56D). In case of blood lysate, it could successfully detect all 0.1 copy/μL 6 out of 6 replicates and 4 out of 6 replicates for 0.01 copy/μL. To begin with, we compared the physical properties, such as the shape of the dried blood matrix, between whole blood and blood lysate. Once we dried the two materials, we cut the bottom of the tube and examined the surface (FIGS. 60A-60E). In the case of whole blood, it did not create a hollow space in the center, whereas blood lysate formed a large hole in the middle of the structure. This can be explained by the coffee ring effect, where drying occurs faster at the edges and leaves a stain around the perimeter.^{[19]} However, in the case of whole blood, this effect is reduced due to compaction towards the inside caused by coagulation factors such as platelets (FIGS. 60B and 60D). Additionally, whole blood is a multi-component agent, minimizing the coffee ring effect.^{[20]} Due to the absence of a hole in the center, the dried blood matrix created from whole blood requires a longer diffusion distance, significantly limiting sensitivity.

Conversely, biphasic LAMP in the dried blood matrix from blood lysate can be easily achieved because the reagents can reach the target DNA without significant diffusion distance. This highlights that material properties can have a substantial impact on the limit of detection (LOD) and optimizing the starting material could be a solution for current blood diagnostics. As a result, faster amplification (i.e., reduced threshold times) were achieved when using blood lysate as a starting material for blood drying (104-10 copies/μL concentration), as well as 1,000 times higher sensitivity (from 10 to 0.01 copies/μL). This suggests that using less thick, homogeneous, and platelet-free blood can lead to highly sensitive detection. Interestingly, we verify that for dried blood lysate, sensitivity of the biphasic assay is not compromised under the absence of the heating step 2 process. To be specific, dried blood matrix with (66.2% porosity) and without (11.3% porosity) heating step 2 has shown the same LOD of ˜1 cfu/mL for E. coli and MRSA, meaning that in case of blood lysate, there is no need for heating step 2 and high porosity for diffusion of reagents. This suggests that material properties, rather than porosity, can have a more significant impact on assay sensitivity. However, it is important to note that this sample processing can result in target molecule loss, and therefore, optimization of the biphasic protocol should be based on the specific target molecule and assay purpose.

In addition to the three properties mentioned, there are other characteristics that can affect sensitivity. For example, the difference in density between whole blood (985 g/μL) and blood lysate (946 g/μL) can impact the evaporation rate (FIGS. 58F and 58G), with blood lysate evaporating at a higher rate due to its lower density (63% evaporation compared to 54% for whole blood). This evaporation difference can result in a higher concentration of target molecules in blood lysate, increasing the chances of successful amplification. Another study has reported that a lower hematocrit (HCT) value and a lower ratio of red blood cells (RBCs) in whole blood may result in increased cracking and subsequently higher surface area when the blood is dried.^{[21]} This finding suggests that blood lysate samples may exhibit greater sensitivity due to the increased surface area.

The biphasic method offers versatility and potential as a diagnostic tool for detecting viruses such as HBV, HCV, and HIV. For example, viruses with a thinner envelop covering genetic material compared to bacteria, such as HBV, can be detected directly using simple blood drying. This is because the process of drying at high temperature can have the additional effect of viral lysis and release of DNA, resulting in both DNA extraction and inhibitor inactivation (purification). This provides advantages over current HBV diagnostic methods, which typically require processing whole blood through centrifugation to obtain plasma or serum samples since centrifugation can result in the loss of a significant amount of HBV due to its low density.^{[22]} However, processing RNA, which is highly sensitive to heat treatment, presents a significant challenge. The use of high temperatures, such as 95° C., during blood drying intended to expedite the drying process could damage genetic materials of interest. Reduction in drying time for minimizing the damage could leave the blood undried due to inadequate drying time and cause the dried blood matrix to resuspend into the liquid phase, decreasing amplification efficiency due to inhibitors and interfering with fluorescence readings. As the volume of the sample increases, drying time is also prolonged, making high temperatures necessary to speed up the process. To detect RNA-based viruses, it is necessary to use RNA-preserving agents and perform drying at low temperatures and new approaches would have to developed to address this important need.

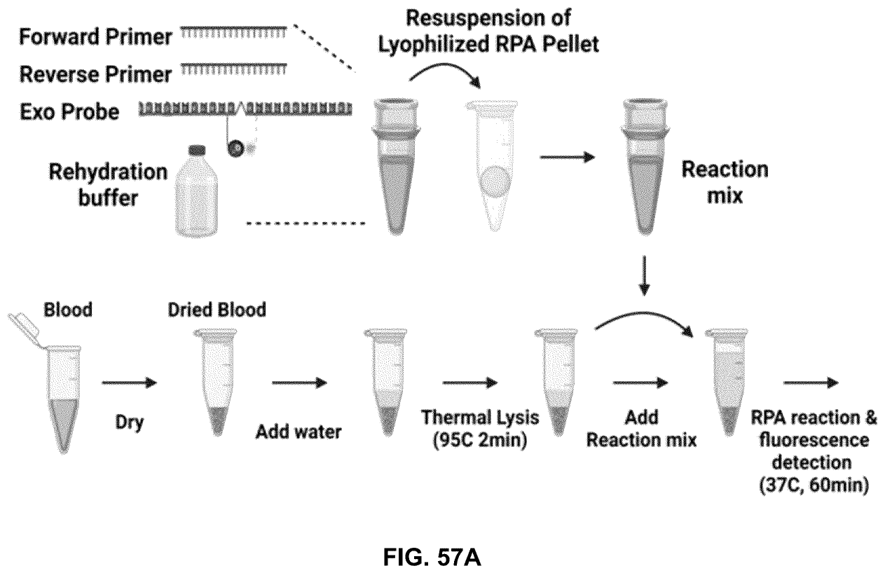

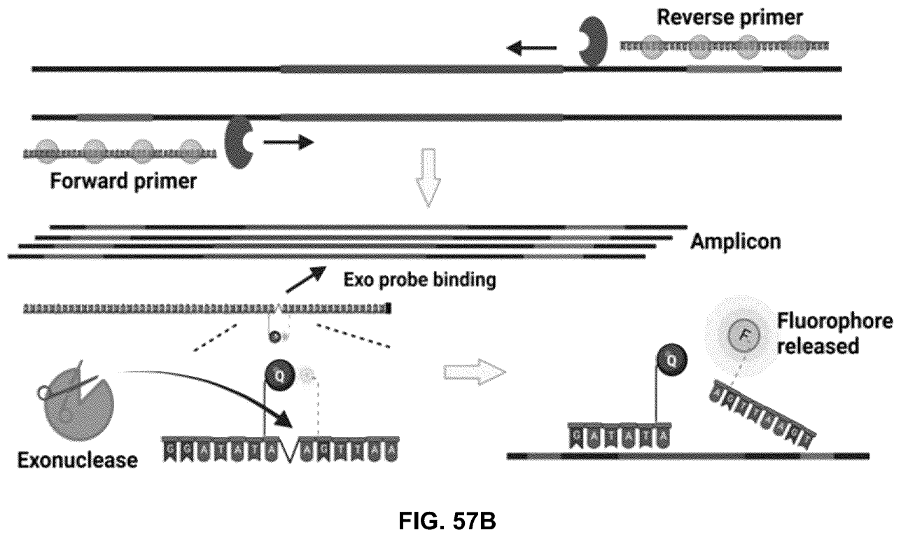

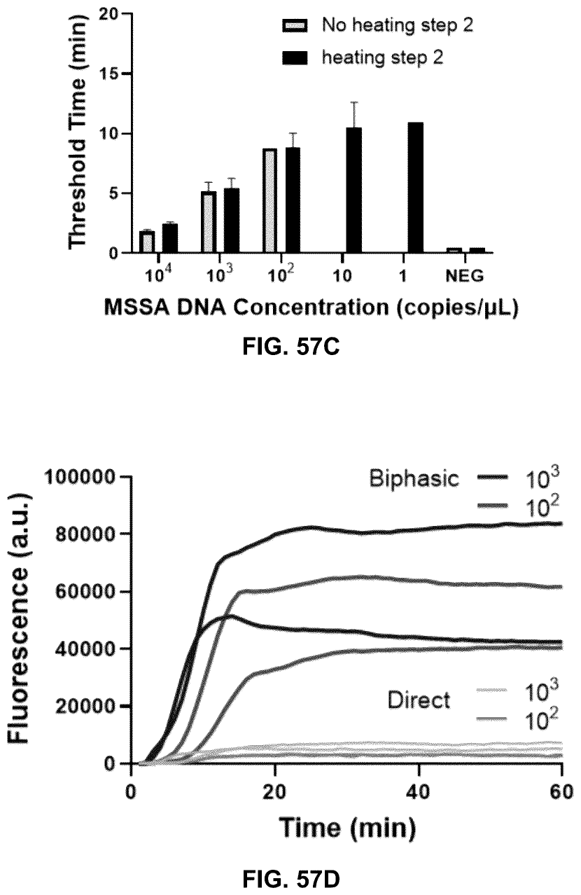

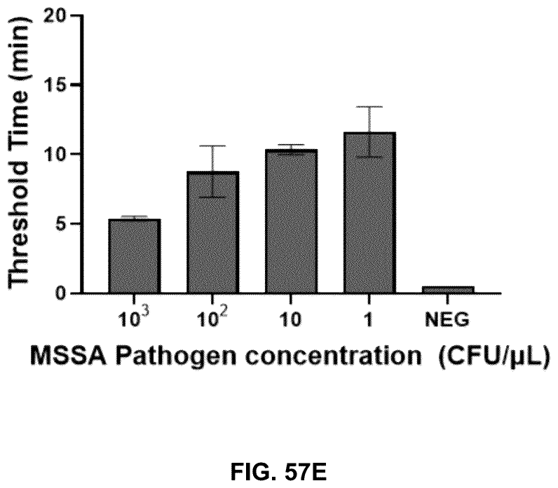

Application of Biphasic reaction to RPA: Next, we demonstrate that recombinase polymerase amplification (RPA) is compatible our biphasic protocol. RPA is also an isothermal amplification process operating at 37° C. and can be used at point the of care or in low resource setting a with simple heater.^{[23]} We utilized the commercial RPA Exo kit from TwistDX company and adapted it for biphasic reaction. We performed heating step 2 only with nuclease-free water and did not include any buffer component from the RPA kit (FIG. 57A). The reaction master mix from the RPA Exo kit, comprising forward and reverse primers, Exo probe, and rehydration buffer, was added to the sample prepared through heating step 2. The RPA reaction with fluorescence detection was carried out at 37° C. for 60 minutes. RPA employs a recombinase-primer complex to target the homologous sequence, similar to the mechanism used by PCR.^{[24]} However, RPA requires an Exo probe for real-time fluorescence detection, as conventional Taq-Man probes cannot be used due to Taq polymerase's exonuclease activity.^{[25]} This activity progressively digests the displaced strand during the strand displacement process, inhibiting DNA amplification. The Exo probe contains an abasic residue that serves as a substrate for exonuclease, which can cleave tetrahydrofuran (THF) only after binding to the target sequence. This cleavage separates the fluorophore (F) from its quencher (Q), allowing for fluorescence detection (FIG. 57B). Utilizing the biphasic protocol with the RPA method, the amplification threshold time of Methicillin-sensitive Staphylococcus aureus (MSSA) DNA spiked in whole blood was determined and depicted in FIG. 57C.^{[16]} The influence of heating step 2 on the biphasic RPA assay was evaluated by testing MSSA DNA spiked in whole blood with and without heating step 2. The detection sensitivity increased to a single copy when heating step 2 was employed, compared to 100 copies/μL without heating step 2. The advantage of utilizing the biphasic method over the direct RPA without blood drying was demonstrated in FIG. 57D, where two concentrations of MSSA DNA (1,000 and 100 copies/μL) were used, and their fluorescence levels were compared. The direct RPA assay using whole blood exhibited minimal fluorescence increment even after amplification due to the interference of red blood cells (RBC) with the fluorescence reading. Conversely, a fluorescence difference ten times higher was shown in the biphasic RPA assay because the fluorescence reading was performed in the clear liquid phase. Finally, the biphasic RPA assay successfully amplified the MSSA pathogen spiked in whole blood at a concentration of 1 colony-forming unit per μL without any nonspecific amplification for the negative control.

The LAMP method is advantageous for use in the biphasic reaction due to its optimal conditions. Firstly, it is an isothermal amplification technique that does not damage the dried blood matrix and does not release the inactivated inhibitors from the structure (FIGS. 54C and 54E). Additionally, it uses a robust Bst polymerase that can function at higher concentrations of inhibitors compared to other techniques such as PCR or RPA.^{[26]} The tolerance of the Bst polymerase is not limited to blood inhibitors and can process crude samples such as nasal swabs and saliva.^{[27,28]} Therefore, the biphasic approach can be extended to other specimens that form a solid-phase structure for the diffusion of amplification reagents.^{[13]} RPA, on the other hand, has some drawbacks due to its assay format and the use of lyophilized products and viscous reagents, limiting their diffusion within the dried blood matrix. Moreover, additional steps such as adding Magnesium Acetate with centrifugation may make the automation of the assay difficult. Regardless, we show the compatibility of RPA with our biphasic protocol.

In summary, we further characterize the biphasic reaction mechanism with a focus on understanding the inhibitor inactivation to improve assay sensitivity. Our findings showed that most inhibitors exist in the solid phase of the dried blood matrix, as measured using LESA-MS. We also observed that the biphasic treatment efficiently purifies the liquid phase, resulting in low inhibitor levels even when compared to highly diluted blood samples. We optimized various parameters, such as drying time, diffusion distance, and material properties, to enhance assay sensitivity using the biphasic protocol. Also, the study demonstrated a novel application of the biphasic reaction in recombinase polymerase amplification (RPA), expanding its potential use in general nucleic acid amplification techniques. An added benefit of the biphasic method that bypasses the extraction and purification is that the entire sample processing steps only require a simple heating instrument, reducing both cost and instrumentation complexity. We believe that application of this solution to existing methods will have widespread use in the detection of various blood-borne diseases, including bacteria and viruses, and could usher in a new era of blood diagnostics.

Materials and Methods

Blood Preparation: Whole venous blood samples were purchased from the vendor BIOIVT using the HUMANWBK2-0101184, Human Whole Blood K2EDTA Gender Unspecified. After collection, the blood samples were kept in a sample rotisserie at 4° C. until used for experiments. Tenfold serial dilutions of DNA or bacterial stocks were performed to achieve the appropriate concentration.

Biphasic Protocol: Pre-Treatment, Heating Step 1 (Drying) and Heating Step 2

The pre-treatment of whole blood to obtain blood lysate was performed following the protocol developed by Ganguli et al. RBC lysis was achieved using ACK lysis buffer (ThermoFisher Scientific), while pathogen mechanical lysis was carried out using 100 μm glass disruptor beads (Scientific Industries, Inc.). In brief, 800 μL (or 1 mL) of blood was mixed with 40 μL (90 mg) of glass disruptor beads and 600 μL of ACK lysis buffer in a 1.5 mL tube. The blood and lysis buffer were mixed by manual pipetting and left to incubate at room temperature for 5 minutes. After centrifugation at 6000 g for 10 minutes, the lysed blood supernatant was removed, and 200 μL of TE buffer was added to the tube for bead lysis. The tubes were vortexed at 3000 rpm for 10 minutes, followed by a brief 10-second centrifugation. The resulting material, i.e., blood lysate, was stored at 4° C. until further use.

For drying, either whole blood or blood lysate (30 μL) was heated at 95° C. for 10 minutes in PCR tubes, followed by heating step 2 after adding water at 95° C. for 2 minutes. Inhibitor characterization was performed using mainly water, but buffer mix components without polymerase and primers were used for heating step 2 in the LAMP reactions.

Hemoglobin Assay: The detection of hemoglobin was carried out using the Hemoglobin Assay Kit (ab234046, abcam), which utilizes colorimetric analysis based on the manufacturer's protocol. Firstly, the Hemoglobin Standard provided in the kit was used to generate a standard curve. Subsequently, 20 μL of each sample was added to individual wells in a 96-well plate, followed by the addition of 180 μL of Hemoglobin Detector to form a color complex. The absorbance was measured at 575 nm using the Synergy™ HT (BioTek) Microplate Reader, and the hemoglobin concentration was calculated using the standard curve. For dried blood samples, 96 μL of water was added and incubated for 10 minutes before measuring 20 μL of the resulting solution. For the 20× and 100× diluted samples, 20 μL of the sample was directly added to the 96-well plate.

Immunoglobulin (IgG) Assay: The detection of IgG was performed using the Human IgG ELISA Kit (ab195215, abeam) according to the manufacturer's protocol. Firstly, a standard curve was generated using the serially diluted standards provided in the kit. Next, 50 μL of each sample was added to individual wells in a 96-well plate, followed by the addition of 50 μL of Antibody Cocktail to each well. The plate was then incubated at room temperature on a plate shaker set to 400 rpm for 40 minutes. Following three washes with 1× Wash Buffer PT, 100 μL of TMB Development Solution was added, and the plate was incubated for 5 minutes in the dark on a plate shaker set to 400 rpm. Subsequently, 100 μL of Stop Solution was added to each well, and the plate was shaken for 1 minute. The absorbance at 450 nm was measured using the Synergy™ HT (BioTek)Microplate Reader. For dried blood samples, 96 μL of water was added and incubated for 10 minutes before measuring 50 μL of the resulting solution. For the 20× and 100× diluted samples, 50 μL of the sample was directly added to the 96-well plate.

Liquid Extraction Surface Analysis Mass Spectrometry: LESA-MS was performed by TriVersa NanoMate LESA® (Advion, Inc., USA) coupled with a Thermo Scientific Q Exactive mass spectrometer. The dried water, supernatant, and dried blood matrix on slide was attached onto the LESA universal adaptor plate. The image was acquired using an Epson Perfection V370 photo scanner with 300 DPI resolution. Advion ChipSoftX software was used to select sample spots and edit sampling methods. A solution of 50% acetonitrile in H2O with 0.1% formic acid was used for extraction and ionization. All solvents and regents are LC-MS grade. For surface extraction, 7.0 μL solvent was aspirated from solvent reservoir. Then a robotic arm moved solvent above the sample spot, and dispensed 4.0 μL was onto surface at height set as −0.3 mm. The solvent stayed for 4 seconds there and 4.5 μL was aspirated at −0.5 mm height. The dispense-aspirate cycle was repeated once before infused into the mass spectrometer via chip-based electrospray ionization through an HD_A_384 nano ESI chip (Advion, Inc). An airgap was applied prior to engaging chip. The gas pressure was set at 1.00 psi and voltage was 1.70 kv at positive mode. For mass spectrometry, spectra were acquired in positive full scan mode at range m/z 600-4000. The capillary temperature was set at 200° C. The resolution was set as 140,000 at 200 m/z. AGC target was set as 1e6, and the maximum injection time was 100 ms. Each scan was composed by 2 microscans.

Poisson Statistics: Given a certain number of target DNA molecules (m) distributed across a dried blood matrix (n), the probability of the matrix containing k copies of the targets can be calculated using the Poisson distribution (Pr(X=k)). The expected value of this distribution corresponds to the average occupancy rate (λ), which is defined as the ratio of the number of target molecules (m) to the volume of dried blood matrix (n).

$$λ = \frac{m}{n} \\ Pr ( X = k ) = \frac{{e}^{- λ} {λ}^{k}}{k !}$$

To determine the probability of at least one amplification occurring in the dried blood matrix, the probability of no molecule being allocated should be subtracted from 1. This can be mathematically expressed as:

Pr(at least one amplification)=1−Pr(X=0)=1−e^{-λ}

Thus, the probability of at least one amplification occurring is a function of the average occupancy rate (λ), which can be influenced by factors such as the drying process and subsequent weight loss.

DNA and Bacteria: The genomic DNA of MRSA strain HFH-30106, NR-10320, was acquired from BEI Resources, and subsequently aliquoted for experimentation or diluted to the appropriate concentration in either whole blood or blood lysate. Similarly, the genomic DNA of E. coli (O157:H7), NR-4629, was obtained from BEI Resources and prepared accordingly. For experiments involving pathogenic bacteria, MSSA strain MN8 was obtained from BEI Resources, and all stocks were stored at −80° C.

Primer Sequences: All primer sequences for the LAMP and RPA reactions were synthesized by Integrated DNA Technology. Primer sequence information is shown in Table 3 of U.S. Pat. App. 63/525,600 filed Jul. 7, 2023. The primer sequences employed in the detection of Escherichia coli mal B gene and MRSA mec A gene via LAMP were sourced from previously published LAMP primers, as documented in references.^{[29,30]} Additionally, the primer sequence used for the detection of MSSA vicK gene through RPA was obtained from previously published RPA primers, as documented in reference.^{[16]}, Primer information: Lamp primers are provided for E. Coli and for MRSA. RPA primers are provided for MSSA. See, e.g., U.S. Pat. App. 63/525,600, which is specifically incorporated by reference for the primer sequences provided therein.

LAMP Reactions: The LAMP assay includes several components, including a final concentration of 1× isothermal amplification buffer (New England Biolabs), 1.025 mmol/L of each deoxyribonucleoside triphosphate (dNTP), 4 mmol/L of MgSO₄ (New England Biolabs), and 0.29 mol/L of Betaine (Sigma-Aldrich). Each component is stored according to the manufacturer's instructions, and a fresh mix containing all components is prepared prior to each reaction. In addition to the buffer components, the reaction also includes 0.15 μM of F3 and B3, 1.17 μM of FIP and BIP, and 0.59 μM of LoopF and LoopB primers, 0.47 U/μL of Bst 2.0 WarmStart DNA Polymerase (New England Biolabs), 1 mg/mL of BSA (New England Biolabs), and 0.74× of EvaGreen (Biotium), a double-stranded DNA intercalating dye. In the biphasic reaction, the total final reaction volume is 96 μL, consisting of 72 μL of buffer mix and 24 μL of reaction mix. After drying the blood, the buffer mix is added for wet heating step 2 at 95° C. for 2 minutes, followed by the addition of the reaction mix for the final LAMP biphasic reaction. LAMP reactions were performed using 0.2-mL PCR tubes in the QuantStudio 3 system (Applied Biosciences) at a constant temperature of 65° C. for 60 minutes. Fluorescence data were measured and recorded every minute. Normalization of the raw fluorescence data was performed, and the amplification threshold time was determined by identifying the point at which 20% of the normalized fluorescence threshold was reached. For the 10 μL×3 tubes reactions, 1/3 of the reaction volume (32 μL, composed of 24 μL of buffer mix and 8 μL of reaction mix) was used in each tube to maintain a consistent volume ratio between the blood sample and reaction mix. The results from the three tubes were then combined to form one set of samples and compared with the 30 μL×1 tube sample.

RPA Reactions: A commercial RPA kit, TwistAmp® exo, was purchased from TwistDx (United Kingdom), and the RPA assay was conducted as per the manufacturer's manual with some adaptations. Firstly, in a 1.5-mL tube, 2.3 μL of forward and reverse primers (10 μM), 0.7 μL of exo probe, and 32.3 μL of rehydration buffer were added and mixed manually before transferring to the lyophilized RPA pellet for resuspension. Following the preparation of the dried blood matrix, water (instead of buffer mix) was added for heating step 2 at 95° C. for 2 minutes, followed by the addition of the reaction mix. For the sample, 20 μL of whole blood was used with 8 minutes of drying at 95° C., followed by adding 15 μL of water for heating step 2 and 35 μL of reaction mix. Before loading the sample, 2.5 μL of 280 mM Magnesium Acetate (MgOAc) was added to the lid of the PCR tubes and centrifuged. The RPA reactions were performed using 0.2-mL PCR tubes in the QuantStudio 3 system (Applied Biosciences) at a constant temperature of 37° C. for 60 minutes. Fluorescence data were measured and recorded every minute. Normalization of the raw fluorescence data was carried out, and the amplification threshold time was determined by identifying the point at which 20% of the normalized fluorescence threshold was reached.

Bacterial Culture: The media and agar plates were obtained from the Cell Media Facility at the University of Illinois Urbana-Champaign (UIUC). Tryptic soy broth and agar were utilized for the culture of MSSA. Bacteria were grown in the broth at 37° C. for 16 hours overnight, following which PBS stocks were prepared. The PBS stocks of pathogens were prepared as follows: 250 μL of the overnight culture was centrifuged at 5,000×g for 10 minutes to create a bacterial pellet, which was then washed twice with 1×PBS. Finally, the bacterial pellet was diluted in 1 mL of PBS, and the resulting solution was aliquoted and kept at room temperature. Each PBS stock was used for a maximum of 4 days after culture.

### References Associated with Example 6

[1] Z. Zhang, M. B. Kermekchiev, W. M. Barnes, J. Mol. Diagnostics 2010, 12, 152.
[2] J. M. Robertson, J. Walsh-Weller, An introduction to PCR primer design and optimization of amplification reactions., Vol. 98, 1998.
[3] M. J. Espy, J. R. Uhl, L. M. Sloan, S. P. Buckwalter, M. F. Jones, E. A. Vetter, J. D. C. Yao, N. L. Wengenack, J. E. Rosenblatt, F. R. Cockerill, T. F. Smith, Clin. Microbiol. Rev. 2006, 19, 595.
[4] D. E. Zak, A. Penn-Nicholson, T. J. Scriba, E. Thompson, S. Suliman, L. M. Amon, H. Mahomed, M. Erasmus, W. Whatney, G. D. Hussey, D. Abrahams, F. Kafaar, T. Hawkridge, S. Verver, E. J. Hughes, M. Ota, J. Sutherland, R. Howe, H. M. Dockrell, W. H. Boom, B. Thiel, T. H. M. Ottenhoff, H. Mayanja-Kizza, A. C. Crampin, K. Downing, M. Hatherill, J. Valvo, S. Shankar, S. K. Panda, S. H. E. Kaufmann, G. Walzl, A. Aderem, W. A. Hanekom, Lancet 2016, 387, 2312.
[5] Y. Bu, H. Huang, G. Zhou, Anal. Biochem. 2008, 375, 370.
[6] D. Cai, O. Behrmann, F. Hufert, G. Dame, G. Urban, Sci. Rep. 2018, 8, 2.
[7] I. A. Nanayakkara, W. Cao, I. M. White, Anal. Chem. 2017, 89, 3773.
[8] C. Katevatis, A. Fan, C. M. Klapperich, PLoS One 2017, 12, 1.
[9] M. Sidstedt, J. Hedman, E. L. Romsos, L. Waitara, L. Wadsö, C. R. Steffen, P. M. Vallone, P. Rådström, Anal. Bioanal. Chem. 2018, 410, 2569.
[10] W. A. Al-Soud, P. Rådström, J. Clin. Microbiol. 2001, 39, 485.
[11] A. Ganguli, J. Lim, A. Mostafa, C. Saavedra, A. Rayabharam, N. R. Aluru, M. Wester, K. C. White, J. Kumar, R. McGuffin, A. Frederick, E. Valera, R. Bashir, Proc. Natl. Acad. Sci. U.S.A 2022, 119, 1.
[12] J. Lim, R. Stavins, V. Kindratenko, J. Baek, L. Wang, K. White, J. Kumar, E. Valera, W. P. King, R. Bashir, Lab Chip 2022, 22, 1297.
[13] A. Mostafa, A. Ganguli, J. Berger, A. Rayabharam, C. Saavedra, N. R. Aluru, R. Bashir, Biotechnol. Bioeng. 2021, 118, 4516.
[14] R. L. Griffiths, A. Dexter, A. J. Creese, H. J. Cooper, Analyst 2015, 140, 6879.
[15] N. J. Martin, R. L. Griffiths, R. L. Edwards, H. J. Cooper, J. Am. Soc. Mass Spectrom. 2015, 26, 1320.
[16] M. Schulz, S. Calabrese, F. Hausladen, H. Wurm, D. Drossart, K. Stock, A. M. Sobieraj, F. Eichenseher, M. J. Loessner, M. Schmelcher, A. Gerhardts, U. Goetz, M. Handel, A. Serr, G. Haecker, J. Li, M. Specht, P. Koch, M. Meyer, P. Tepper, R. Rother, M. Jehle, S. Wadle, R. Zengerle, F. Von Stetten, N. Paust, N. Borst, Lab Chip 2020, 20, 2549.
[17] R. A. Lee, H. De Puig, P. Q. Nguyen, N. M. Angenent-Mari, N. M. Donghia, J. P. McGee, J. D. Dvorin, C. M. Klapperich, N. R. Pollock, J. J. Collins, Proc. Natl. Acad. Sci. U.S.A 2020, 117, 25722.
[18] Y. Ning, X. Cui, C. Yang, F. Jing, X. Bian, L. Yi, G. Li, Anal. Chim. Acta 2019, 1055, 65.
[19] R. D. Deegan, O. Bakajin, T. F. Dupont, 1997, 827.
[20] B. J. De Gans, U. S. Schubert, Langmuir 2004, 20, 7789.
[21] R. Iqbal, A. Q. Shen, A. K. Sen, J. Colloid Interface Sci. 2020, 579, 541.
[22] M. Ghosh, S. Nandi, S. Dutta, M. K. Saha, World J. Hepatol. 2015, 7, 2482.
[23] L. Lillis, J. Siverson, A. Lee, J. Cantera, M. Parker, O. Piepenburg, D. A. Lehman, D. S. Boyle, Mol. Cell. Probes 2016, 30, 74.
[24] R. K. Daher, G. Stewart, M. Boissinot, M. G. Bergeron, Clin. Chem. 2016, 62, 947.
[25] J. S. McQuillan, M. W. Wilson, Lett. Appl. Microbiol. 2021, 72, 382.
[26] L. Becherer, N. Borst, M. Bakheit, S. Frischmann, R. Zengerle, F. Von Stetten, Anal. Methods 2020, 12, 717.
[27] I. Park, J. Lim, S. You, M. T. Hwang, J. Kwon, 1.
[28] E. Valera, A. Jankelow, J. Lim, V. Kindratenko, A. Ganguli, K. White, J. Kumar, R. Bashir, ACS Nano 2021, 15, 7899.
[29] J. Hill, S. Beriwal, I. Chandra, V. K. Paul, A. Kapil, T. Singh, R. M. Wadowsky, V. Singh, A. Goyal, T. Jahnukainen, J. R. Johnson, P. I. Tarr, A. Vats, J. Clin. Microbiol. 2008, 46, 2800.
[30] Z. Xu, L. Li, J. Chu, B. M. Peters, M. L. Harris, B. Li, L. Shi, M. E. Shirtliff, Food Res. Int. 2012, 47, 166.
[31] G. L. Lukacs, P. Haggie, O. Seksek, D. Lechardeur, N. Freedman, A. S. Verkman, J. Biol. Chem. 2000, 275, 1625.

### Example 7: Pixelation and Electric Field Lysing

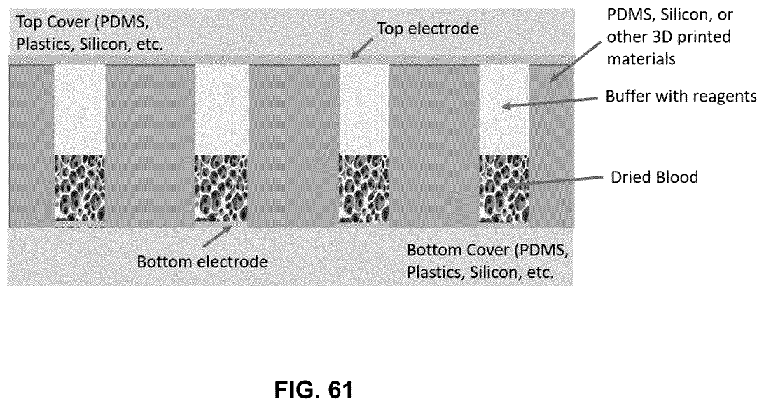

FIGS. 61-65 illustrate configurations where the whole blood sample is pixelated into a plurality of discrete islands with electric field lysing. In this manner, chemical lysis of blood components may be avoided or both chemical end electric field lysing may be used.

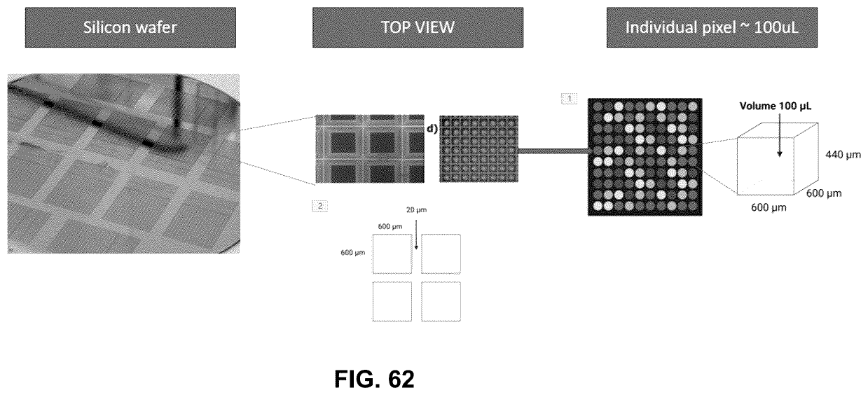

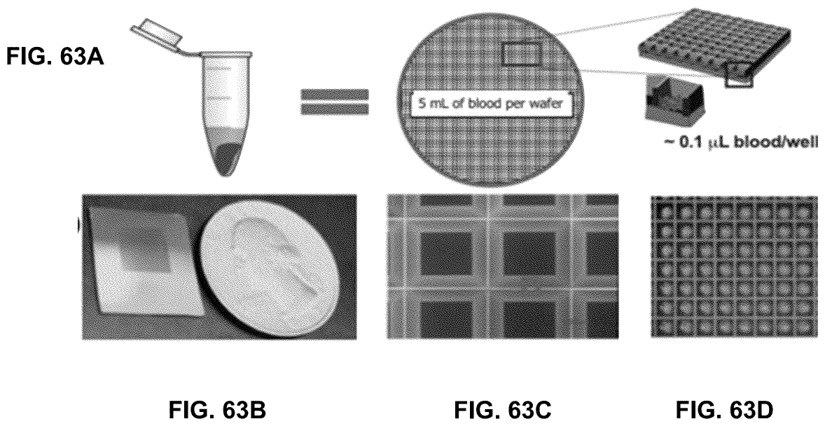

Referring to FIG. 61, a blood sample is applied to substrate having a bottom electrode and a top electrode. The substrate may be etched Si, PDMS or a 3D printed material. Basically, any material that can support a plurality of microwells is compatible with the instant platform. The blood sample is dried and pixelated into a plurality of dried blood sample islands, with one dried blood sample island per well. A buffer with reagents (referred herein more generally as “liquid having a reagent”) is applied to the dried blood. Energization of the electrodes results in an electric field that can be configured to lyse a blood component. This may occur before or after the blood drying step. FIGS. 62-63 illustrates the microwell and pixelation configuration.

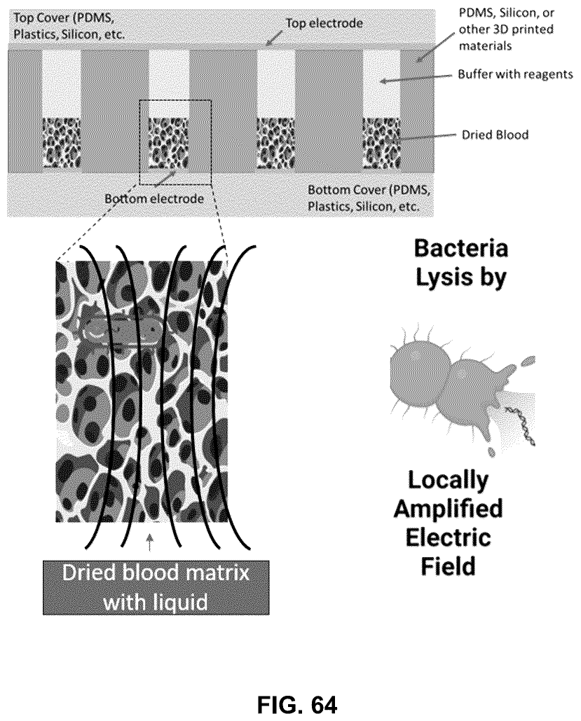

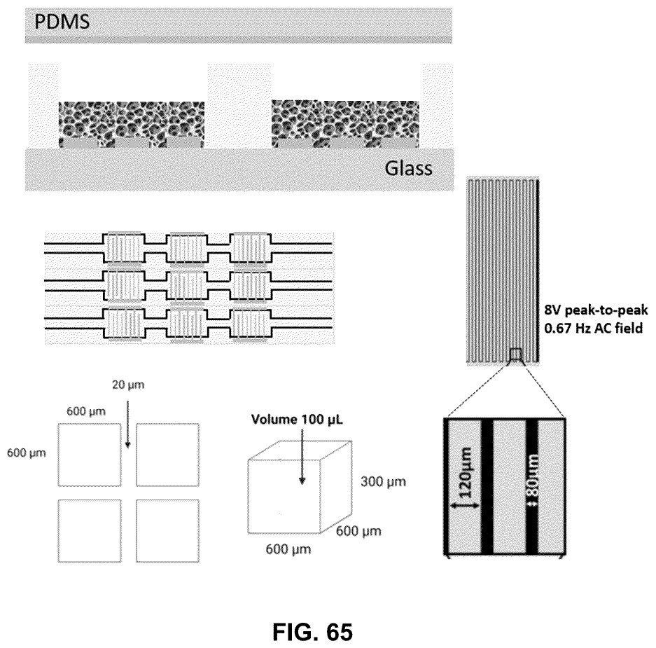

FIG. 64 is a further schematic of FIG. 61, illustrated the applied electric field in the dried blood matrix having the liquid and attendant bacterial lysis by the locally amplified electric field. Accordingly, the method may be further described as producing a locally amplified electric field by introducing the liquid, preferably a buffer liquid having an ionic strength to amplify the electric field, to the dried blood sample island, thereby ensuring pathogen lysis and/or lysis of a blood component. FIG. 65 illustrates another electrode configuration, wherein a plurality of spaced electrodes are matched to dried biological islands, including underneath wells. This example illustrates the electrodes are each 120 μm in width, running the length of the island (600 μm) with separation distance between adjacent electrodes of 80 μm, so that there are three electrodes beneath the island. In this manner, an AC field may be applied, such as a 0.67 Hz AC field with 8V peak-to-peak. Accordingly, any of the methods and devices provided herein may match to each dried sample island a plurality of spaced apart microelectrodes that provide an AC field with a user-controllable frequency and magnitude tailored to provide the desired lysing.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, are disclosed separately. When a Markush group or other grouping is used herein, such as compositions, physical dimensions or temperatures, all individual members of the group and all combinations and sub-combinations possible of the group are intended to be individually included in the disclosure. Specific names of compounds are intended to be exemplary, as it is known that one of ordinary skill in the art can name the same compounds differently.

Every formulation or combination of components described or exemplified herein can be used to practice the invention, unless otherwise stated.

Whenever a range is given in the specification, for example, a temperature range, a time range, a volume range, a ratio range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

We claim:

1. A method of detecting one or more target analytes in a blood sample, the method comprising the steps of:
applying a blood sample to a substrate;
thermally treating the blood sample to dry the blood sample to generate a dried blood sample island having a fluidic network inside the dried blood sample island;
applying a liquid having a reagent suspended in a liquid buffer to the dried blood sample island, wherein the reagent is used for nucleic acid detection, and the liquid transits the fluidic network to access a nucleic acid in the dried blood sample island;
diffusing a nucleic acid from the dried blood sample island to a supernatant liquid having the applied reagent;
detecting a presence or absence of the nucleic acid that is a target nucleic acid in the dried blood sample island by a bi-phasic reaction, that occurs in both the dried blood sample island and in the supernatant liquid having the reagent suspended in the liquid buffer;
thereby detecting the one or more target analytes in the blood sample.

2. The method of claim 1, further comprising the step of:
pixelating the blood sample into a plurality of blood sample islands, wherein the plurality of blood sample islands are provided in an array configuration.

3. The method of claim 1, further comprising the step of:
applying an electric field to the dried blood sample island to electrically lyse a biological material and release nucleic acid from the biological material, wherein the biological material is optionally a pathogen selected from the group consisting of: bacteria, virus, a fungus, and a cell having a marker for a disease condition such as a cancer cell.

4. The method of claim 1, wherein the detecting is by electrical or optical detection.

5. The method of claim 1, wherein a heme background interference is confined to the dried blood sample island.

6. The method of claim 1, wherein the reagent comprises amplification enzymes and primers selected to amplify a target nucleic acid sequence of the target analyte.

7. The method of claim 6, wherein the target nucleic acid is amplified by the reagent into amplicons in the dried blood sample island, wherein the amplicons diffuse from the dried blood sample island to the supernatant liquid that covers the dried blood sample island.

8. The method of claim 7, wherein the amplicons are detected by a change in fluorescence of the supernatant.

9. The method of claim 7, wherein the amplified step is by an isothermal reaction, including a loop-mediated isothermal amplification reaction or a recombinase polymerase amplification (RPA); or a CRISPR-based amplification.

10. The method of claim 1, wherein the target analyte corresponds to one or more pathogens.

11. The method of claim 1, configured to have a limit of detection (LOD) of the target analyte in whole blood as small as 1 cfu per blood sample starting volume, optionally a blood sample starting volume of up to 10 mL.

12. The method of claim 1, wherein the target analyte is detected in a total test time that is less than 2.5 hours.

13. The method of claim 1, wherein the blood sample is from unprocessed whole blood.

14. The method of claim 1, further comprising the step of lysing the applied blood sample by applying an electric field to the blood sample island(s).

15. The method of claim 14, wherein the blood sample is pixelated into an array of blood sample islands, and the electric field is applied to the array of blood sample islands.

16. The method of claim 1, further comprising the steps of:
applying a red blood cell (RBC) lysis buffer to lyse at least a portion of a population of RBCs in an unprocessed whole blood sample; and
mechanically and/or electrically lysing a pathogen in the whole blood sample, including a pathogen that is a bacteria.

17. The method of claim 1, wherein the thermally treating step comprises:
elevating a temperature to between 65° C. and 97° C. for an elevation time of between 1 minute and 20 minutes to generate a porosity in the resultant dried blood sample islands for access by the reagents and diffusion of an amplified target nucleic acid from an interior of the dried blood sample to the liquid supernatant positioned outside the dried blood sample; wherein the temperature and elevation time are selected to achieve a porosity in the dried blood sample between 30% and 85%, including between 50% and 65%.

18. The method of claim 1, further comprising the step of:
applying an electric field after the liquid is introduced to the dried blood sample to lyse at least a portion of the dried blood sample island; and
optionally applying a chemical lysing agent to the dried blood sample after the step of applying the electric field.

19. The method of claim 18, wherein the step of applying the electric field comprises energizing an electrode pair comprising:
a first electrode positioned underneath a substrate that supports the dried blood sample; and
a second electrode positioned above or in the supernatant liquid.

20. The method of claim 18, wherein the dried blood sample with the liquid in the fluidic network is configured to have one or more localized regions resulting in high local electric field to increase lysing efficiency as compared to fluid regions without the dried blood sample.

21. The method of claim 1, wherein the detecting the one or more target analytes is used to select an antibiotic treatment therapy in an individual requiring treatment of a blood stream infection, wherein the detecting the one or more target analytes is within 3 hours of the individual initially presenting with a blood infection symptom.

22. A method of determining a presence or an absence of a pathogen in an individual that may have a blood stream infection, the method comprising the steps of:
obtaining a whole blood sample from the individual;
thermally treating the whole blood sample to generate a dried blood sample island having a fluidic network inside the dried blood sample;
lysing RBCs in the whole blood sample and mechanically or electrically lysing a pathogen, if present, in the whole blood sample to generate a lysed blood sample;
introducing a liquid buffer having amplification reagents to the dried blood sample island, wherein the amplification reagents diffuse through the fluidic network to contact an interior portion of the dried blood sample;
amplifying a target nucleic acid, if present, to generate amplicons in the interior portion of the dried blood sample;
diffusing the amplicons from the interior portion of the dried blood sample to a liquid supernatant that surrounds the dried blood sample; and
optically detecting amplicons in the liquid supernatant, wherein a positive optical detection corresponds to presence of the pathogen in the whole blood sample and a negative optical detection corresponds to absence of the pathogen in the whole blood sample;
thereby determining the presence or the absence of the pathogen.

23. The method of claim 22, wherein the pathogen corresponds to a bacteria, a fungus, and/or a virus, and the bacteria is optionally an antibiotic-resistant or an antibiotic-susceptible bacteria.

24. The method of claim 22, further comprising the step of:
pixelating the lysed blood sample into a plurality of blood sample islands, wherein the plurality of blood sample islands are arranged in an array configuration.

25. The method of claim 22, wherein the lysing step comprises applying an electric field after the liquid buffer is introduced to the dried blood sample to lyse at least a portion of the dried blood sample island and at least a portion of any pathogens therein.

26. The method of claim 25, further comprising the step of chemically lysing at least a portion of the whole blood sample and/or the dried blood sample.
